SEQUENCE LISTING (32 sequences)

SEQ ID NO: 1 - DNA, 13 bp, artificial
  Oligonucleotide sequence motif
gcattggtattca

SEQ ID NO: 2 - DNA, 12 bp, artificial
  Oligonucleotide sequence motif
gttgacactgtc

SEQ ID NO: 3 - DNA, 13 bp, artificial
  LNA antisense gapmer oligonucleotide
gcattggtattca

SEQ ID NO: 4 - DNA, 13 bp, artificial
  LNA antisense gapmer oligonucleotide conjugate
gcattggtattca

SEQ ID NO: 5 - DNA, 13 bp, artificial
  LNA antisense gapmer oligonucleotide conjugate
gcattggtattca

SEQ ID NO: 6 - DNA, 16 bp, artificial
  LNA antisense gapmer oligonucleotide conjugate
tcagcattggtattca

SEQ ID NO: 7 - DNA, 15 bp, artificial
  LNA antisense gapmer oligonucleotide conjugate
cagcattggtattca

SEQ ID NO: 8 - DNA, 14 bp, artificial
  LNA antisense gapmer oligonucleotide
agcattggtattca

SEQ ID NO: 9 - DNA, 16 bp, artificial
  LNA antisense gapmer oligonucleotide conjugate
tcagcattggtattca

SEQ ID NO: 10 - DNA, 15 bp, artificial
  LNA antisense gapmer oligonucleotide conjugate
cagcattggtattca

SEQ ID NO: 11 - DNA, 14 bp, artificial
  LNA antisense gapmer oligonucleotide
agcattggtattca

SEQ ID NO: 12 - DNA, 16 bp, artificial
  LNA antisense gapmer oligonucleotide conjugate
gacgcattggtattca

SEQ ID NO: 13 - DNA, 13 bp, artificial
  LNA antisense gapmer oligonucleotide conjugate
gcattggtattca

SEQ ID NO: 14 - DNA, 13 bp, artificial
  LNA antisense gapmer oligonucleotide conjugate
gcattggtattca

SEQ ID NO: 15 - DNA, 13 bp, artificial
  LNA antisense gapmer oligonucleotide conjugate
gcattggtattca

SEQ ID NO: 16 - DNA, 15 bp, artificial
  LNA antisense gapmer oligonucleotide conjugate
cagcattggtattca

SEQ ID NO: 17 - DNA, 13 bp, artificial
  LNA antisense gapmer oligonucleotide conjugate
gcattggtattca

SEQ ID NO: 18 - DNA, 13 bp, artificial
  LNA antisense gapmer oligonucleotide conjugate
gcattggtattca

SEQ ID NO: 19 - DNA, 15 bp, artificial
  LNA antisense gapmer oligonucleotide conjugate
cagcattggtattca

SEQ ID NO: 20 - DNA, 13 bp, artificial
  LNA antisense gapmer oligonucleotide conjugate
gcattggtattca

SEQ ID NO: 21 - DNA, 13 bp, artificial
  LNA antisense gapmer oligonucleotide conjugate
gcattggtattca

SEQ ID NO: 22 - DNA, 13 bp, artificial
  LNA antisense gapmer oligonucleotide conjugate
gcattggtattca

SEQ ID NO: 23 - DNA, 15 bp, artificial
  LNA antisense gapmer oligonucleotide conjugate
cagcattggtattca

SEQ ID NO: 24 - DNA, 13 bp, artificial
  LNA antisense gapmer oligonucleotide conjugate
gcattggtattca

SEQ ID NO: 25 - DNA, 13 bp, artificial
  LNA antisense gapmer oligonucleotide conjugate
gcattggtattca

SEQ ID NO: 26 - DNA, 15 bp, artificial
  LNA antisense gapmer oligonucleotide conjugate
cagcattggtattca

SEQ ID NO: 27 - DNA, 12 bp, artificial
  LNA antisense gapmer oligonucleotide
gttgacactgtc

SEQ ID NO: 28 - DNA, 14 bp, artificial
  LNA antisense gapmer oligonucleotide
cagttgacactgtc

SEQ ID NO: 29 - DNA, 12 bp, artificial
  LNA antisense gapmer oligonucleotide
gttgacactgtc

SEQ ID NO: 30 - DNA, 13 bp, artificial
  LNA antisense gapmer oligonucleotide conjugate
gcattggtattca

SEQ ID NO: 31 - DNA, 12 bp, artificial
  LNA antisense gapmer oligonucleotide
gttgacactgtc

SEQ ID NO: 32 - DNA, 14121 bp, Homo sapiens
attcccaccgggacctgcggggctgagtgcccttctcggttgctgccgctgaggagcccgcccagccagccagggccgcgaggccgaggccaggccgcagcccaggagccgccccaccgcagctggcgatggacccgccgaggcccgcgctgctggcgctgctggcgctgcctgcgctgctgctgctgctgctggcgggcgccagggccgaagaggaaatgctggaaaatgtcagcctggtctgtccaaaagatgcgacccgattcaagcacctccggaagtacacatacaactatgaggctgagagttccagtggagtccctgggactgctgattcaagaagtgccaccaggatcaactgcaaggttgagctggaggttccccagctctgcagcttcatcctgaagaccagccagtgcaccctgaaagaggtgtatggcttcaaccctgagggcaaagccttgctgaagaaaaccaagaactctgaggagtttgctgcagccatgtccaggtatgagctcaagctggccattccagaagggaagcaggttttcctttacccggagaaagatgaacctacttacatcctgaacatcaagaggggcatcatttctgccctcctggttcccccagagacagaagaagccaagcaagtgttgtttctggataccgtgtatggaaactgctccactcactttaccgtcaagacgaggaagggcaatgtggcaacagaaatatccactgaaagagacctggggcagtgtgatcgcttcaagcccatccgcacaggcatcagcccacttgctctcatcaaaggcatgacccgccccttgtcaactctgatcagcagcagccagtcctgtcagtacacactggacgctaagaggaagcatgtggcagaagccatctgcaaggagcaacacctcttcctgcctttctcctacaagaataagtatgggatggtagcacaagtgacacagactttgaaacttgaagacacaccaaagatcaacagccgcttctttggtgaaggtactaagaagatgggcctcgcatttgagagcaccaaatccacatcacctccaaagcaggccgaagctgttttgaagactctccaggaactgaaaaaactaaccatctctgagcaaaatatccagagagctaatctcttcaataagctggttactgagctgagaggcctcagtgatgaagcagtcacatctctcttgccacagctgattgaggtgtccagccccatcactttacaagccttggttcagtgtggacagcctcagtgctccactcacatcctccagtggctgaaacgtgtgcatgccaacccccttctgatagatgtggtcacctacctggtggccctgatccccgagccctcagcacagcagctgcgagagatcttcaacatggcgagggatcagcgcagccgagccaccttgtatgcgctgagccacgcggtcaacaactatcataagacaaaccctacagggacccaggagctgctggacattgctaattacctgatggaacagattcaagatgactgcactggggatgaagattacacctatttgattctgcgggtcattggaaatatgggccaaaccatggagcagttaactccagaactcaagtcttcaatcctgaaatgtgtccaaagtacaaagccatcactgatgatccagaaagctgccatccaggctctgcggaaaatggagcctaaagacaaggaccaggaggttcttcttcagactttccttgatgatgcttctccgggagataagcgactggctgcctatcttatgttgatgaggagtccttcacaggcagatattaacaaaattgtccaaattctaccatgggaacagaatgagcaagtgaagaactttgtggcttcccatattgccaatatcttgaactcagaagaattggatatccaagatctgaaaaagttagtgaaagaagctctgaaagaatctcaacttccaactgtcatggacttcagaaaattctctcggaactatcaactctacaaatctgtttctcttccatcacttgacccagcctcagccaaaatagaagggaatcttatatttgatccaaataactaccttcctaaagaaagcatgctgaaaactaccctcactgcctttggatttgcttcagctgacctcatcgagattggcttggaaggaaaaggctttgagccaacattggaagctctttttgggaagcaaggatttttcccagacagtgtcaacaaagctttgtactgggttaatggtcaagttcctgatggtgtctctaaggtcttagtggaccactttggctataccaaagatgataaacatgagcaggatatggtaaatggaataatgctcagtgttgagaagctgattaaagatttgaaatccaaagaagtcccggaagccagagcctacctccgcatcttgggagaggagcttggttttgccagtctccatgacctccagctcctgggaaagctgcttctgatgggtgcccgcactctgcaggggatcccccagatgattggagaggtcatcaggaagggctcaaagaatgacttttttcttcactacatcttcatggagaatgcctttgaactccccactggagctggattacagttgcaaatatcttcatctggagtcattgctcccggagccaaggctggagtaaaactggaagtagccaacatgcaggctgaactggtggcaaaaccctccgtgtctgtggagtttgtgacaaatatgggcatcatcattccggacttcgctaggagtggggtccagatgaacaccaacttcttccacgagtcgggtctggaggctcatgttgccctaaaagctgggaagctgaagtttatcattccttccccaaagagaccagtcaagctgctcagtggaggcaacacattacatttggtctctaccaccaaaacggaggtgatcccacctctcattgagaacaggcagtcctggtcagtttgcaagcaagtctttcctggcctgaattactgcacctcaggcgcttactccaacgccagctccacagactccgcctcctactatccgctgaccggggacaccagattagagctggaactgaggcctacaggagagattgagcagtattctgtcagcgcaacctatgagctccagagagaggacagagccttggtggataccctgaagtttgtaactcaagcagaaggtgcgaagcagactgaggctaccatgacattcaaatataatcggcagagtatgaccttgtccagtgaagtccaaattccggattttgatgttgacctcggaacaatcctcagagttaatgatgaatctactgagggcaaaacgtcttacagactcaccctggacattcagaacaagaaaattactgaggtcgccctcatgggccacctaagttgtgacacaaaggaagaaagaaaaatcaagggtgttatttccataccccgtttgcaagcagaagccagaagtgagatcctcgcccactggtcgcctgccaaactgcttctccaaatggactcatctgctacagcttatggctccacagtttccaagagggtggcatggcattatgatgaagagaagattgaatttgaatggaacacaggcaccaatgtagataccaaaaaaatgacttccaatttccctgtggatctctccgattatcctaagagcttgcatatgtatgctaatagactcctggatcacagagtccctcaaacagacatgactttccggcacgtgggttccaaattaatagttgcaatgagctcatggcttcagaaggcatctgggagtcttccttatacccagactttgcaagaccacctcaatagcctgaaggagttcaacctccagaacatgggattgccagacttccacatcccagaaaacctcttcttaaaaagcgatggccgggtcaaatataccttgaacaagaacagtttgaaaattgagattcctttgccttttggtggcaaatcctccagagatctaaagatgttagagactgttaggacaccagccctccacttcaagtctgtgggattccatctgccatctcgagagttccaagtccctacttttaccattcccaagttgtatcaactgcaagtgcctctcctgggtgttctagacctctccacgaatgtctacagcaacttgtacaactggtccgcctcctacagtggtggcaacaccagcacagaccatttcagccttcgggctcgttaccacatgaaggctgactctgtggttgacctgctttcctacaatgtgcaaggatctggagaaacaacatatgaccacaagaatacgttcacactatcatgtgatgggtctctacgccacaaatttctagattcgaatatcaaattcagtcatgtagaaaaacttggaaacaacccagtctcaaaaggtttactaatattcgatgcatctagttcctggggaccacagatgtctgcttcagttcatttggactccaaaaagaaacagcatttgtttgtcaaagaagtcaagattgatgggcagttcagagtctcttcgttctatgctaaaggcacatatggcctgtcttgtcagagggatcctaacactggccggctcaatggagagtccaacctgaggtttaactcctcctacctccaaggcaccaaccagataacaggaagatatgaagatggaaccctctccctcacctccacctctgatctgcaaagtggcatcattaaaaatactgcttccctaaagtatgagaactacgagctgactttaaaatctgacaccaatgggaagtataagaactttgccacttctaacaagatggatatgaccttctctaagcaaaatgcactgctgcgttctgaatatcaggctgattacgagtcattgaggttcttcagcctgctttctggatcactaaattcccatggtcttgagttaaatgctgacatcttaggcactgacaaaattaatagtggtgctcacaaggcgacactaaggattggccaagatggaatatctaccagtgcaacgaccaacttgaagtgtagtctcctggtgctggagaatgagctgaatgcagagcttggcctctctggggcatctatgaaattaacaacaaatggccgcttcagggaacacaatgcaaaattcagtctggatgggaaagccgccctcacagagctatcactgggaagtgcttatcaggccatgattctgggtgtcgacagcaaaaacattttcaacttcaaggtcagtcaagaaggacttaagctctcaaatgacatgatgggctcatatgctgaaatgaaatttgaccacacaaacagtctgaacattgcaggcttatcactggacttctcttcaaaacttgacaacatttacagctctgacaagttttataagcaaactgttaatttacagctacagccctattctctggtaactactttaaacagtgacctgaaatacaatgctctggatctcaccaacaatgggaaactacggctagaacccctgaagctgcatgtggctggtaacctaaaaggagcctaccaaaataatgaaataaaacacatctatgccatctcttctgctgccttatcagcaagctataaagcagacactgttgctaaggttcagggtgtggagtttagccatcggctcaacacagacatcgctgggctggcttcagccattgacatgagcacaaactataattcagactcactgcatttcagcaatgtcttccgttctgtaatggccccgtttaccatgaccatcgatgcacatacaaatggcaatgggaaactcgctctctggggagaacatactgggcagctgtatagcaaattcctgttgaaagcagaacctctggcatttactttctctcatgattacaaaggctccacaagtcatcatctcgtgtctaggaaaagcatcagtgcagctcttgaacacaaagtcagtgccctgcttactccagctgagcagacaggcacctggaaactcaagacccaatttaacaacaatgaatacagccaggacttggatgcttacaacactaaagataaaattggcgtggagcttactggacgaactctggctgacctaactctactagactccccaattaaagtgccacttttactcagtgagcccatcaatatcattgatgctttagagatgagagatgccgttgagaagccccaagaatttacaattgttgcttttgtaaagtatgataaaaaccaagatgttcactccattaacctcccattttttgagaccttgcaagaatattttgagaggaatcgacaaaccattatagttgtactggaaaacgtacagagaaacctgaagcacatcaatattgatcaatttgtaagaaaatacagagcagccctgggaaaactcccacagcaagctaatgattatctgaattcattcaattgggagagacaagtttcacatgccaaggagaaactgactgctctcacaaaaaagtatagaattacagaaaatgatatacaaattgcattagatgatgccaaaatcaactttaatgaaaaactatctcaactgcagacatatatgatacaatttgatcagtatattaaagatagttatgatttacatgatttgaaaatagctattgctaatattattgatgaaatcattgaaaaattaaaaagtcttgatgagcactatcatatccgtgtaaatttagtaaaaacaatccatgatctacatttgtttattgaaaatattgattttaacaaaagtggaagtagtactgcatcctggattcaaaatgtggatactaagtaccaaatcagaatccagatacaagaaaaactgcagcagcttaagagacacatacagaatatagacatccagcacctagctggaaagttaaaacaacacattgaggctattgatgttagagtgcttttagatcaattgggaactacaatttcatttgaaagaataaatgacgttcttgagcatgtcaaacactttgttataaatcttattggggattttgaagtagctgagaaaatcaatgccttcagagccaaagtccatgagttaatcgagaggtatgaagtagaccaacaaatccaggttttaatggataaattagtagagttggcccaccaatacaagttgaaggagactattcagaagctaagcaatgtcctacaacaagttaagataaaagattactttgagaaattggttggatttattgatgatgctgtcaagaagcttaatgaattatcttttaaaacattcattgaagatgttaacaaattccttgacatgttgataaagaaattaaagtcatttgattaccaccagtttgtagatgaaaccaatgacaaaatccgtgaggtgactcagagactcaatggtgaaattcaggctctggaactaccacaaaaagctgaagcattaaaactgtttttagaggaaaccaaggccacagttgcagtgtatctggaaagcctacaggacaccaaaataaccttaatcatcaattggttacaggaggctttaagttcagcatctttggctcacatgaaggccaaattccgagagaccctagaagatacacgagaccgaatgtatcaaatggacattcagcaggaacttcaacgatacctgtctctggtaggccaggtttatagcacacttgtcacctacatttctgattggtggactcttgctgctaagaaccttactgactttgcagagcaatattctatccaagattgggctaaacgtatgaaagcattggtagagcaagggttcactgttcctgaaatcaagaccatccttgggaccatgcctgcctttgaagtcagtcttcaggctcttcagaaagctaccttccagacacctgattttatagtccccctaacagatttgaggattccatcagttcagataaacttcaaagacttaaaaaatataaaaatcccatccaggttttccacaccagaatttaccatccttaacaccttccacattccttcctttacaattgactttgtagaaatgaaagtaaagatcatcagaaccattgaccagatgctgaacagtgagctgcagtggcccgttccagatatatatctcagggatctgaaggtggaggacattcctctagcgagaatcaccctgccagacttccgtttaccagaaatcgcaattccagaattcataatcccaactctcaaccttaatgattttcaagttcctgaccttcacataccagaattccagcttccccacatctcacacacaattgaagtacctacttttggcaagctatacagtattctgaaaatccaatctcctcttttcacattagatgcaaatgctgacatagggaatggaaccacctcagcaaacgaagcaggtatcgcagcttccatcactgccaaaggagagtccaaattagaagttctcaattttgattttcaagcaaatgcacaactctcaaaccctaagattaatccgctggctctgaaggagtcagtgaagttctccagcaagtacctgagaacggagcatgggagtgaaatgctgttttttggaaatgctattgagggaaaatcaaacacagtggcaagtttacacacagaaaaaaatacactggagcttagtaatggagtgattgtcaagataaacaatcagcttaccctggatagcaacactaaatacttccacaaattgaacatccccaaactggacttctctagtcaggctgacctgcgcaacgagatcaagacactgttgaaagctggccacatagcatggacttcttctggaaaagggtcatggaaatgggcctgccccagattctcagatgagggaacacatgaatcacaaattagtttcaccatagaaggacccctcacttcctttggactgtccaataagatcaatagcaaacacctaagagtaaaccaaaacttggtttatgaatctggctccctcaacttttctaaacttgaaattcaatcacaagtcgattcccagcatgtgggccacagtgttctaactgctaaaggcatggcactgtttggagaagggaaggcagagtttactgggaggcatgatgctcatttaaatggaaaggttattggaactttgaaaaattctcttttcttttcagcccagccatttgagatcacggcatccacaaacaatgaagggaatttgaaagttcgttttccattaaggttaacagggaagatagacttcctgaataactatgcactgtttctgagtcccagtgcccagcaagcaagttggcaagtaagtgctaggttcaatcagtataagtacaaccaaaatttctctgctggaaacaacgagaacattatggaggcccatgtaggaataaatggagaagcaaatctggatttcttaaacattcctttaacaattcctgaaatgcgtctaccttacacaataatcacaactcctccactgaaagatttctctctatgggaaaaaacaggcttgaaggaattcttgaaaacgacaaagcaatcatttgatttaagtgtaaaagctcagtataagaaaaacaaacacaggcattccatcacaaatcctttggctgtgctttgtgagtttatcagtcagagcatcaaatcctttgacaggcattttgaaaaaaacagaaacaatgcattagattttgtcaccaaatcctataatgaaacaaaaattaagtttgataagtacaaagctgaaaaatctcacgacgagctccccaggacctttcaaattcctggatacactgttccagttgtcaatgttgaagtgtctccattcaccatagagatgtcggcattcggctatgtgttcccaaaagcagtcagcatgcctagtttctccatcctaggttctgacgtccgtgtgccttcatacacattaatcctgccatcattagagctgccagtccttcatgtccctagaaatctcaagctttctcttccagatttcaaggaattgtgtaccataagccatatttttattcctgccatgggcaatattacctatgatttctcctttaaatcaagtgtcatcacactgaataccaatgctgaactttttaaccagtcagatattgttgctcatctcctttcttcatcttcatctgtcattgatgcactgcagtacaaattagagggcaccacaagattgacaagaaaaaggggattgaagttagccacagctctgtctctgagcaacaaatttgtggagggtagtcataacagtactgtgagcttaaccacgaaaaatatggaagtgtcagtggcaacaaccacaaaagcccaaattccaattttgagaatgaatttcaagcaagaacttaatggaaataccaagtcaaaacctactgtctcttcctccatggaatttaagtatgatttcaattcttcaatgctgtactctaccgctaaaggagcagttgaccacaagcttagcttggaaagcctcacctcttacttttccattgagtcatctaccaaaggagatgtcaagggttcggttctttctcgggaatattcaggaactattgctagtgaggccaacacttacttgaattccaagagcacacggtcttcagtgaagctgcagggcacttccaaaattgatgatatctggaaccttgaagtaaaagaaaattttgctggagaagccacactccaacgcatatattccctctgggagcacagtacgaaaaaccacttacagctagagggcctctttttcaccaacggagaacatacaagcaaagccaccctggaactctctccatggcaaatgtcagctcttgttcaggtccatgcaagtcagcccagttccttccatgatttccctgaccttggccaggaagtggccctgaatgctaacactaagaaccagaagatcagatggaaaaatgaagtccggattcattctgggtctttccagagccaggtcgagctttccaatgaccaagaaaaggcacaccttgacattgcaggatccttagaaggacacctaaggttcctcaaaaatatcatcctaccagtctatgacaagagcttatgggatttcctaaagctggatgtaaccaccagcattggtaggagacagcatcttcgtgtttcaactgcctttgtgtacaccaaaaaccccaatggctattcattctccatccctgtaaaagttttggctgataaattcattattcctgggctgaaactaaatgatctaaattcagttcttgtcatgcctacgttccatgtcccatttacagatcttcaggttccatcgtgcaaacttgacttcagagaaatacaaatctataagaagctgagaacttcatcatttgccctcaacctaccaacactccccgaggtaaaattccctgaagttgatgtgttaacaaaatattctcaaccagaagactccttgattcccttttttgagataaccgtgcctgaatctcagttaactgtgtcccagttcacgcttccaaaaagtgtttcagatggcattgctgctttggatctaaatgcagtagccaacaagatcgcagactttgagttgcccaccatcatcgtgcctgagcagaccattgagattccctccattaagttctctgtacctgctggaattgtcattccttcctttcaagcactgactgcacgctttgaggtagactctcccgtgtataatgccacttggagtgccagtttgaaaaacaaagcagattatgttgaaacagtcctggattccacatgcagctcaaccgtacagttcctagaatatgaactaaatgttttgggaacacacaaaatcgaagatggtacgttagcctctaagactaaaggaacatttgcacaccgtgacttcagtgcagaatatgaagaagatggcaaatatgaaggacttcaggaatgggaaggaaaagcgcacctcaatatcaaaagcccagcgttcaccgatctccatctgcgctaccagaaagacaagaaaggcatctccacctcagcagcctccccagccgtaggcaccgtgggcatggatatggatgaagatgacgacttttctaaatggaacttctactacagccctcagtcctctccagataaaaaactcaccatattcaaaactgagttgagggtccgggaatctgatgaggaaactcagatcaaagttaattgggaagaagaggcagcttctggcttgctaacctctctgaaagacaacgtgcccaaggccacaggggtcctttatgattatgtcaacaagtaccactgggaacacacagggctcaccctgagagaagtgtcttcaaagctgagaagaaatctgcagaacaatgctgagtgggtttatcaaggggccattaggcaaattgatgatatcgacgtgaggttccagaaagcagccagtggcaccactgggacctaccaagagtggaaggacaaggcccagaatctgtaccaggaactgttgactcaggaaggccaagccagtttccagggactcaaggataacgtgtttgatggcttggtacgagttactcaagaattccatatgaaagtcaagcatctgattgactcactcattgattttctgaacttccccagattccagtttccggggaaacctgggatatacactagggaggaactttgcactatgttcataagggaggtagggacggtactgtcccaggtatattcgaaagtccataatggttcagaaatactgttttcctatttccaagacctagtgattacacttcctttcgagttaaggaaacataaactaatagatgtaatctcgatgtatagggaactgttgaaagatttatcaaaagaagcccaagaggtatttaaagccattcagtctctcaagaccacagaggtgctacgtaatcttcaggaccttttacaattcattttccaactaatagaagataacattaaacagctgaaagagatgaaatttacttatcttattaattatatccaagatgagatcaacacaatcttcagtgattatatcccatatgtttttaaattgttgaaagaaaacctatgccttaatcttcataagttcaatgaatttattcaaaacgagcttcaggaagcttctcaagagttacagcagatccatcaatacattatggcccttcgtgaagaatattttgatccaagtatagttggctggacagtgaaatattatgaacttgaagaaaagatagtcagtctgatcaagaacctgttagttgctcttaaggacttccattctgaatatattgtcagtgcctctaactttacttcccaactctcaagtcaagttgagcaatttctgcacagaaatattcaggaatatcttagcatccttaccgatccagatggaaaagggaaagagaagattgcagagctttctgccactgctcaggaaataattaaaagccaggccattgcgacgaagaaaataatttctgattaccaccagcagtttagatataaactgcaagatttttcagaccaactctctgattactatgaaaaatttattgctgaatccaaaagattgattgacctgtccattcaaaactaccacacatttctgatatacatcacggagttactgaaaaagctgcaatcaaccacagtcatgaacccctacatgaagcttgctccaggagaacttactatcatcctctaattttttaaaagaaatcttcatttattcttcttttccaattgaactttcacatagcacagaaaaaattcaaactgcctatattgataaaaccatacagtgagccagccttgcagtaggcagtagactataagcagaagcacatatgaactggacctgcaccaaagctggcaccagggctcggaaggtctctgaactcagaaggatggcattttttgcaagttaaagaaaatcaggatctgagttattttgctaaacttgggggaggaggaacaaataaatggagtctttattgtgtatcata

(19) United States
(12) Patent Application Publication
(10) Pub. No.: US 2016/0289677 A1
(43) Pub. Date: Oct. 6, 2016

(54) APOB Antisense Conjugate Compounds

(71) Applicant: ROCHE INNOVATION CENTER COPENHAGEN A/S, Hørsholm (DK)

(72) Inventors: Nanna Albaek, Birkerød (DK); Henrik Frydenlund Hansen, Ringsted (DK); Susanne Kammler, Holte (DK); Marie Lindholm, Malmo (SE); Mark Turner, Hoersholm (DK); Henrik Orum, Vaerlose (DK)

(73) Assignee: Roche Innovation Center Copenhagen A/S, Horsholm (DK)

(21) Appl. No.: 15/036,218

(22) PCT Filed: Nov. 14, 2014
(86) PCT No.: PCT/EP2014/074554

§ 371 Date: May 12, 2016

(30) Foreign Application Priority Data
Nov. 14, 2013 (EP) 13192930.9
Nov. 14, 2013 (EP) PCT/EP2013/073858
Nov. 14, 2013 (EP) PCT/EP2013/073859
Jan. 30, 2014 (EP) 14153266.3
May 12, 2014 (EP) 14167879.7

(51) Int. Cl. C12N 15/113

(52) U.S. Cl. CPC C12N 15/113; C12N 2310/11; C12N 2310/351; C12N 2320/32; C12N 2310/315

(57) ABSTRACT

The present invention relates to conjugates of antisense oligonucleotides (oligomers) that target the APOB gene at position 2265 to 2277.

### FIELD OF INVENTION

The present invention relates to conjugates of LNA antisense oligonucleotides (oligomers) that target ApoB.

### BACKGROUND

Apolipoprotein B (also known as ApoB, apolipoprotein B-100; ApoB-100, apolipoprotein B-48; ApoB-48 and Ag(x) antigen), is a large glycoprotein that serves an indispensable role in the assembly and secretion of lipids and in the transport and receptor-mediated uptake and delivery of distinct classes of lipoproteins. ApoB plays an important role in the regulation of circulating lipoprotein levels, and is therefore relevant in terms of atherosclerosis susceptibility, which is highly correlated with the ambient concentration of apolipoprotein B-containing lipoproteins. See Davidson and Shelness (Annul Rev. Nutr., 2000, 20, 169-193) for further details of the two forms of ApoB present in mammals, their structure and medicinal importance of ApoB.

Elevated plasma levels of the ApoB-100-containing lipoprotein Lp(a) are associated with increased risk for atherosclerosis and its manifestations, which may include hypercholesterolemia (Seed et al., N. Engl. J. Med., 1990, 322, 1494-1499), myocardial infarction (Sandkamp et al., Clin. Chew., 1990, 36, 20-23), and thrombosis (Nowak-Gottl et al., Pediatrics, 1997, 99, Eli).

The plasma concentration of Lp(a) is strongly influenced by heritable factors and is refractory to most drug and dietary manipulation (Katan and Beynen, Am. J. Epidemiol., 1987, 125, 387-399; Vessby et al., Atherosclerosis, 1982, 44, 61-71). Pharmacologic therapy of elevated Lp(a) levels has been only modestly successful and apheresis remains the most effective therapeutic modality (Hajjar and Nachman, Annul Rev. Med., 1996, 47, 423-442).

Two forms of apolipoprotein B exist in mammals. ApoB-100 represents the full-length protein containing 4536 amino acid residues synthesized exclusively in the human liver (Davidson and Shelness, Annul Rev. Nutr., 2000, 20, 169-193). A truncated form known as ApoB-48 is colinear with the amino terminal 2152 residues and is synthesized in the small intestine of all mammals (Davidson and Shelness, Annul Rev. Nutr., 2000, 20, 169-193).

The basis by which the common structural gene for apolipoprotein B produces two distinct protein isoforms is a process known as RNA editing. A site specific cytosine-to-uracil editing reaction produces a UAA stop codon and translational termination of apolipoprotein B to produce ApoB-48 (Davidson and Shelness, Annul Rev. Nutr., 2000, 20, 169-193).

The medicinal significance of mammalian ApoB has been verified using transgenic mice studies either over expressing human ApoB (Kim and Young, J. Lipid Res., 1998, 39, 703-723; Nishina et al., J. Lipid Res., 1990, 31, 859-869) or ApoB knock-out mice (Farese et al., Proc. Natl. Acad. Sci. U.S.A, 1995, 92, 1774-1778; Kim and Young, J. Lipid Res., 1998, 39, 703-723).

Strategies aimed at inhibiting apolipoprotein B function have been directed to Lp(a) apheresis, antibodies, antibody fragments and ribozymes. Moreover, antisense oligonucleotides have been disclosed WO 03/97662, WO 03/11887 and WO 2004/44181 WO2007/031081, WO2008/113830, WO2010/142805, and WO2010/076248. SPC3833 and SPC4955 (which have SEQ ID NO 1 and 2) are two LNA compounds which have been previously identified as potent compounds which target human apolipoprotein B (ApoB) mRNA.

WO2007/146511 reports on short bicyclic (LNA) gapmer antisense oligonucleotides which apparently are more potent and less toxic than longer compounds. The exemplified compounds appear to be 14 nts in length,

According to van Poelgeest et al., (American Journal of Kidney Disease, 2013 October; 62(4):796-800), the administration of LNA antisense oligonucleotide SPC5001 in human clinical trials may result in acute kidney injury.

According to EP 1 984 381 B1, Seth et al., Nucleic Acids Symposium Series 2008 No. 52 553-554 and Swayze et al., Nucleic Acid Research 2007, vol 35, pp687-700, LNA oligonucleotides cause significant hepatotoxicity in animals. According to WO2007/146511, the toxicity of LNA oligonucleotides may be avoided by using LNA gapmers as short as 12-14 nucleotides in length. EP 1 984 381B1 recommends using 6′ substituted bicyclic nucleotides to decrease the hepatotoxicity potential of LNA oligonucleotides. According to Hagedorn et al., Nucleic Acid Therapeutics 2013, the hepatotoxic potential of antisense oligonucleotide may be predicted from their sequence and modification pattern.

Oligonucleotide conjugates have been extensively evaluated for use in siRNAs, where they are considered essential in order to obtain sufficient in vivo potency. For example, see WO2004/044141 and WO2009/073809 refers to modified oligomeric compounds that modulate gene expression via an RNA interference pathway. The oligomeric compounds include one or more conjugate moieties that can modify or enhance the pharmacokinetic and pharmacodynamic properties of the attached oligomeric compound.

WO2012/083046, WO2012/089352 and WO2012/089602 reports on a galactose cluster-pharmacokinetic modulator targeting moiety for siRNAs.

In contrast, single stranded antisense oligonucleotides are typically administered therapeutically without conjugation or formulation. The main target tissues for antisense oligonucleotides are the liver and the kidney, although a wide range of other tissues are also accessible by the antisense modality, including lymph node, spleen, and bone marrow.

WO 2005/086775 refers to targeted delivery of therapeutic agents to specific organs using a therapeutic chemical moiety, a cleavable linker and a labeling domain. The cleavable linker may be, for example, a disulfide group, a peptide or a restriction enzyme cleavable oligonucleotide domain.

WO 2011/126937 refers to targeted intracellular delivery of oligonucleotides via conjugation with small molecule ligands.

WO2009/025669 refers to polymeric (polyethylene glycol) linkers containing pyridyl disulphide moieties. See also Zhao et al., Bioconjugate Chem. 2005 16 758-766.

Chaltin et al., Bioconjugate Chem. 2005 16 827-836 reports on cholesterol modified mono- di- and tetrameric oligonucleotides used to incorporate antisense oligonucleotides into cationic liposomes, to produce a dendrimeric delivery system. Cholesterol is conjugated to the oligonucleotides via a lysine linker.

Other non-cleavable cholesterol conjugates have been used to target siRNAs and antagomirs to the liver—see for example, Soutscheck et al., Nature 2004 vol. 432 173-178 and Krützfeldt et al., Nature 2005 vol 438, 685-689. For the partially phosphorothiolated siRNAs and antagomirs, the use of cholesterol as a liver targeting entity was found to be essential for in vivo activity.

There is therefore a need for ApoB targeting LNA antisense compounds have enhanced efficacy and a reduced toxicity risk.

### SUMMARY OF INVENTION

The invention provides for an antisense oligonucleotide conjugate (the compound of the invention) comprising an oligomer with the oligonucleotide motif of SEQ ID NO 2 (region A) covalently linked to an asialoglycoprotein receptor targeting moiety (Region C).

The invention provides for an antisense oligonucleotide conjugate (the compound of the invention) comprising an oligomer with the oligonucleotide motif of SEQ ID NO 2 (region A) covalently linked to a conjugate moiety (Region C) which comprises one or more N-acetylgalactosamine (GalNAc) moieties.

The invention provides for an antisense oligonucleotide conjugate (the compound of the invention) comprising the LNA oligomer of SEQ ID NO 27: 5′ GTtgacactgTC 3′ (region A) covalently linked to a conjugate moiety which comprises a trivalent N-acetylgalactosamine (GalNAc) moiety.

The invention provides for an antisense oligonucleotide conjugate (the compound of the invention) comprising an oligomer with the oligonucleotide motif of SEQ ID NO 2 (region A) covalently linked to a conjugate moiety (region C) which comprises cholesterol moiety, wherein the cholesterol containing conjugate moiety is joined to the oligomer via a biocleavable linker region (region B).

The invention provides an antisense oligonucleotide conjugate comprising the LNA oligomer SEQ ID NO 27: 5′ GₛTₛtₛgₛaₛcₛaₛcₛtₛgₛTₛC 3′ (region A), wherein capital letters represent beta-D-oxy LNA, lower case letters represent DNA nucleosides, LNA cytosines are 5-methyl cytosine, and all internucleoside linkages are phosphorothioate (s), and; a conjugate moiety (region C) comprising an N-acetylgalactosamine moiety or a cholesterol moiety, wherein said conjugate moiety is joined to said LNA oligomer, via a bio cleavable linker (region B).

The invention provides for pharmaceutical composition comprising the compound of the invention, and a pharmaceutically acceptable diluent, carrier, salt or adjuvant.

The invention provides for the compound or pharmaceutical composition of the invention, for use as a medicament. In particular for use in the treatment of acute coronary syndrome, or hypercholesterolemia or related disorder, such as a disorder selected from the group consisting of atherosclerosis, hyperlipidemia, hypercholesterolemia, HDL/LDL cholesterol imbalance, dyslipidemias, e.g., familial hyperlipidemia (FCHL), acquired hyperlipidemia, statin-resistant hypercholesterolemia, coronary artery disease (CAD), and coronary heart disease (CHD).

The invention provides for the compound or pharmaceutical composition of the invention, for use as a medicament in the prevention or reduction of atherosclerotic plaques.

The invention provides for the use of the compound or pharmaceutical composition of the invention, for the manufacture of a medicament for the treatment of acute coronary syndrome, or hypercholesterolemia or a related disorder, such as a disorder selected from the group consisting of atherosclerosis, hyperlipidemia, hypercholesterolemia, HDL/LDL cholesterol imbalance, dyslipidemias, e.g., familial hyperlipidemia (FCHL), acquired hyperlipidemia, statin-resistant hypercholesterolemia, coronary artery disease (CAD), and coronary heart disease (CHD).

The invention provides for a method of treating acute coronary syndrome, or hypercholesterolemia or a related disorder, such as a disorder selected from the group consisting atherosclerosis, hyperlipidemia, hypercholesterolemia, HDL/LDL cholesterol imbalance, dyslipidemias, e.g., familial hyperlipidemia (FCHL), acquired hyperlipidemia, statin-resistant hypercholesterolemia, coronary artery disease (CAD), and coronary heart disease (CHD), said method comprising administering an effective amount of the compound or pharmaceutical composition according to the invention, to a patient suffering from, or likely to suffer from hypercholesterolemia or a related disorder.

The invention provides for an in vivo or in vitro method for the inhibition of ApoB in a cell which is expressing ApoB, said method comprising administering the compound of the invention to said cell so as to inhibit ApoB in said cell.

The invention provides for the compound of the invention for use in medicine, such as for use as a medicament.

### BRIEF DESCRIPTION OF FIGURES

FIG. 1: Non-limiting illustration of oligomers of the invention attached to an activation group (i.e. a protected reactive group—as the third region). The internucleoside linkage L may be, for example phosphodiester, phosphorothioate, phosphorodithioate, boranophosphate or methylphosphonate, such as phosphodiester. PO is a phosphodiester linkage. Compound a) has a region B with a single DNA or RNA, the linkage between the second and the first region is PO. Compound b) has two DNA/RNA (such as DNA) nucleosides linked by a phosphodiester linkage. Compound c) has three DNA/RNA (such as DNA) nucleosides linked by a phosphodiester linkages. In some embodiments, Region B may be further extended by further phosphodiester DNA/RNA (such as DNA nucleosides). The activation group is illustrated on the left side of each compound, and may, optionally be linked to the terminal nucleoside of region B via a phosphorus nucleoside linkage group, such as phosphodiester, phosphorothioate, phosphorodithioate, boranophosphate or methylphosphonate, or in some embodiments a triazole linkage. Compounds d), e), & f) further comprise a linker (Y) between region B and the activation group, and region Y may be linked to region B via, for example, a phosphorus nucleoside linkage group, such as phosphodiester, phosphorothioate, phosphorodithioate, boranophosphate or methylphosphonate, or in some embodiments a triazole linkage.

FIG. 2: Equivalent compounds as shown in FIG. 1; however a reactive group is used in place of the activation group. The reactive group may, in some embodiments be the result of activation of the activation group (e.g. deprotection). The reactive group may, in non-limiting examples, be an amine or alcohol.

FIG. 3: Non-limiting Illustration of compounds of the invention. Same nomenclature as FIG. 1. X may in some embodiments be a conjugate, such as a lipophilic conjugate such as cholesterol, or a asialoglycoprotein receptor targeting moiety such as a galactose cluster or GalNAc comprising moiety or another conjugate such as those described herein. In addition, or alternatively X may be a targeting group or a blocking group. In some aspects X may be an activation group (see FIG. 1), or a reactive group (see FIG. 2). X may be covalently attached to region B via a phosphorus nucleoside linkage group, such as phosphodiester, phosphorothioate, phosphorodithioate, boranophosphate or methylphosphonate, or may be linked via an alternative linkage, e.g. a triazol linkage (see L in compounds d), e), and f)).

FIG. 4. Non-limiting Illustration of compounds of the invention, where the compounds comprise the optional linker between the third region (X) and the second region (region B). Same nomenclature as FIG. 1. Suitable linkers are disclosed herein, and include, for example alkyl linkers, for example C6 linkers. In compounds A, B and C, the linker between X and region B is attached to region B via a phosphorus nucleoside linkage group, such as phosphodiester, phosphorothioate, phosphorodithioate, boranophosphate or methylphosphonate, or may be linked via an alternative linkage e.g. a triazol linkage (Li). In these compounds Lii represents the internucleoside linkage between the first (A) and second regions (B).

FIGS. 5a and b. 5b shows a non-limiting example of a method of synthesis of compounds of the invention. US represent an oligonucleotide synthesis support, which may be a solid support. X is the third region, such as a conjugate, a targeting group, a blocking group etc. In an optional pre-step, X is added to the oligonucleotide synthesis support. Otherwise the support with X already attached may be obtained (i). In a first step, region B is synthesized (ii), followed by region A (iii), and subsequently the cleavage of the oligomeric compound of the invention from the oligonucleotide synthesis support (iv). In an alternative method the pre-step involves the provision of an oligonucleotide synthesis support with a region X and a linker group (Y) attached (see FIG. 5a). In some embodiments, either X or Y (if present) is attached to region B via a phosphorus nucleoside linkage group, such as phosphodiester, phosphorothioate, phosphorodithioate, boranophosphate or methylphosphonate, or an alternative linkage, such as a triazol linkage.

FIG. 6. A non-limiting example of a method of synthesis of compounds of the invention which comprise a linker (Y) between the third region (X) and the second region (B). US represents an oligonucleotide synthesis support, which may be a solid support. X is the third region, such as a conjugate, a targeting group, a blocking group etc. In an optional pre-step, Y is added to the oligonucleotide synthesis support. Otherwise the support with Y already attached may be obtained (i). In a first step, region B is synthesized (ii), followed by region A (iii), and subsequently the cleavage of the oligomeric compound of the invention from the oligonucleotide synthesis support (iv). In some embodiments (as shown), region X may be added to the linker (Y) after the cleavage step (v). In some embodiments, Y is attached to region B via a phosphorus nucleoside linkage group, such as phosphodiester, phosphorothioate, phosphorodithioate, boranophosphate or methylphosphonate, or an alternative linkage, such as a triazol linkage.

FIG. 7. A non-limiting example of a method of synthesis of compounds of the invention which utilize an activation group. In an optional pre-step, the activation group is attached the oligonucleotide synthesis support (i), or the oligonucleotide synthesis support with activation group is otherwise obtained. In step ii) region B is synthesized followed by region A (iii). The oligomer is then cleaved from the oligonucleotide synthesis support (iv). The intermediate oligomer (comprising an activation group) may then be activated (vi) or (viii) and a third region (X) added (vi), optionally via a linker (Y) (ix). In some embodiments, X (or Y when present) is attached to region B via a phosphorus nucleoside linkage group, such as phosphodiester, phosphorothioate, phosphorodithioate, boranophosphate or methylphosphonate, or an alternative linkage, such as a triazol linkage.

FIG. 8. A non-limiting example of a method of synthesis of compounds of the invention, wherein a bifunctional oligonucleotide synthesis support is used (i). In such a method, either the oligonucleotide is synthesized in an initial series of steps (ii)-(iii), followed by the attachment of the third region (optionally via a linker group Y), the oligomeric compound of the invention may then be cleaved (v). Alternatively, as shown in steps (vi)-(ix), the third region (optionally with a linker group (Y) is attached to the oligonucleotide synthesis support (this may be an optional pre-step)—or an oligonucleotide synthesis support with the third region (optionally with Y) is otherwise provided, the oligonucleotide is then synthesized (vii-viii). The oligomeric compound of the invention may then be cleaved (ix). In some embodiments, X (or Y when present) is attached to region B via a phosphorus nucleoside linkage group, such as phosphodiester, phosphorothioate, phosphorodithioate, boranophosphate or methylphosphonate, or an alternative linkage, such as a triazol linkage. The US may in some embodiment, prior to the method (such as the pre-step) comprise a step of adding a bidirectional (bifunctional) group which allows the independent synthesis of the oligonucleotide and the covalent attachment of group X, Y (or X and Y) to support (as shown)—this may for example be achieved using a triazol or of nucleoside group. The bidirectional (bifunctional) group, with the oligomer attached, may then be cleaved from the support.

FIG. 9. A non-limiting example of a method of synthesis of compounds of the invention: In an initial step, the first region (A) is synthesized (ii), followed by region B. In some embodiments the third region is then attached to region B (iii), optionally via a phosphate nucleoside linkage (or e.g. a triazol linkage). The oligomeric compound of the invention may then be cleaved (iv). When a linker(Y) is used, in some embodiments the steps (v)-(viii) may be followed: after synthesis of region B, the linker group (Y) is added, and then either attached to (Y) or in a subsequent step, region X is added (vi). The oligomeric compound of the invention may then be cleaved (vii). In some embodiments, X (or Y when present) is attached to region B via a phosphorus nucleoside linkage group, such as phosphodiester, phosphorothioate, phosphorodithioate, boranophosphate or methylphosphonate, or an alternative linkage, such as a triazol linkage.

FIG. 10. A non-limiting example of a method of synthesis of compounds of the invention: In this method an activation group is used: Steps (i)-(iii) are as per FIG. 9. However after the oligonucleotide synthesis (step iii), an activation group (or a reactive group) is added to region B, optionally via a phosphate nucleoside linkage. The oligonucleotide is then cleaved from the support (v). The activation group may be subsequently activated to produce a reactive group, and then the third region (X), such as the conjugate, blocking group or targeting group, is added to the reactive group (which may be the activated activation group or the reactive group), to produce the oligomer (vi). As shown in (vii)-(viii), after cleavage, a linker group (Y) is added (vii), and then either attached to (Y) or in a subsequent step, region X is added to produce the oligomer (viii). It should be recognized that in an alternative all of the steps (ii)-(viii) may be performed on the oligonucleotide synthesis support, and in such instances a final step of cleaving the oligomer from the support may be performed. In some embodiments, the reactive group or activation group is attached to region B via a phosphorus nucleoside linkage group, such as phosphodiester, phosphorothioate, phosphorodithioate, boranophosphate or methylphosphonate, or an alternative linkage, such as a triazol linkage

FIG. 11. Silencing of ApoB mRNA with Cholesterol-conjugates in vivo. Mice were injected with a single dose of 1 mg/kg unconjugated LNA-antisense oligonucleotide (SEQ ID NO: 3) or equimolar amounts of the LNA antisense oligonucleotides conjugated to Cholesterol with different linkers (see Table 7 for sequences) and sacrificed at days 1, 3, 7 and 10 after dosing. RNA was isolated from liver and kidney and subjected to ApoB specific RT-qPCR A. Quantification of ApoB mRNA from liver samples normalized to GAPDH and shown as percentage of the average of equivalent saline controls B. Quantification of ApoB mRNA from kidney samples normalized to GAPDH and shown as percentage of the average of equivalent saline controls.

FIG. 12. Shows the following conjugation moieties cholesterol C6, FAM, TOC, and Folic acid, which may be used as X-Y- in compounds as illustrated in FIGS. 1 to 4. The wavy line represents the covalent link of the conjugate moiety to the oligomer.

FIG. 12A. Shows GalNAc conjugation moieties which may be used as X-Y- in compounds as illustrated in FIGS. 1 to 4. The wavy line represents the covalent link of the conjugate moiety to the oligomer.

FIG. 12B. Shows some of the specific cholesterol conjugated compounds used in the examples and listed in Table 3. L denote beta-D-oxy-LNA monomers; uppercase letters without the L denote DNA monomers, the subscript “s” denotes a phosphorothioate linkage the superscript “^{Me}C” denotes a beta-D-oxy-LNA monomer containing a 5-methylcytosine base; the subscript “o” denotes phophodiester linkage.

FIG. 12C. Shows some of the specific FAM conjugated compounds used in the examples and listed in Table 4. L denote beta-D-oxy-LNA monomers; uppercase letters without the L denote DNA monomers, the subscript “s” denotes a phosphorothioate linkage the superscript “^{Me}C” denotes a beta-D-oxy-LNA monomer containing a 5-methylcytosine base; the subscript “o” denotes phophodiester linkage.

FIG. 12D. Shows some of the specific folic acid, GalNAc, FAM and TOC conjugated compounds used in the examples and listed in Table 5. L denote beta-D-oxy-LNA monomers; uppercase letters without the L denote DNA monomers, the subscript “s” denotes a phosphorothioate linkage the superscript “^{Me}C” denotes a beta-D-oxy-LNA monomer containing a 5-methylcytosine base; the subscript “o” denotes phophodiester linkage.

FIG. 12E. Shows some of the specific GalNAc conjugated compounds used in the examples and listed in Table 6. L denote beta-D-oxy-LNA monomers; uppercase letters without the L denote DNA monomers, the subscript “s” denotes a phosphorothioate linkage the superscript “^{Me}C” denotes a beta-D-oxy-LNA monomer containing a 5-methylcytosine base; the subscript “o” denotes phophodiester linkage.

FIG. 13: Examples of trivalent GalNAc conjugate moieties which may be used in the present invention. Conjugates 1-4 illustrate 4 suitable GalNAc conjugate moieties, and conjugates 1a-4a refer to the same conjugates with an additional linker moiety (Y) which is used to link the conjugate to the oligomer (region A or to a biocleavable linker, such as region B). The wavy line represents the covalent link of the conjugate moiety to the oligomer.

FIG. 13A: Illustrate GalNAc conjugate 2a conjugated to the oligonucleotide sequence motif of SEQ ID NO 2 and the LNA containing oligonucleotide of SEQ ID NO 27. This corresponds to the compound of SEQ ID NO 29.

FIG. 14: Examples of cholesterol conjugate moieties. The wavy line represents the covalent link of the conjugate moiety to the oligomer.

FIG. 15: In vivo silencing of ApoB mRNA with different conjugates (See example 4). Mice were treated with 1 mg/kg of ASO with different conjugates either without biocleavable linker, with Dithio-linker (SS) or with DNA/PO-linker (PO). RNA was isolated from liver (A) and kidney samples (B) and analysed for ApoB mRNA knock down. Data is shown compared to Saline (=1).

FIG. 16: Example 7—ApoB mRNA expression

FIG. 17: Example 7—Total cholesterol in serum

FIG. 18: Example 7—Oligonucleotide content in liver and kidney

FIG. 19: Serum ApoB and LDL-C levels in monkeys treated with a single dose of SEQ ID NO 28 or 29 at 1.0 or 2.5 mg/kg.

FIG. 20: Serum ApoB and LDL-C levels in monkeys treated with a single dose of SEQ ID NO 7 or 20 at 1.0 or 2.5 mg/kg.

FIG. 21: Total serum cholesterol levels in mice treated with a single iv dose of SEQ ID NO 27, 28 or 29 at 0.1 mg/kg, 0.25 mg/kg or 1.0 mg/kg.

FIG. 22: Serum ApoB and LDL-C levels in monkeys treated with a multiple dose of SEQ ID NO 27, 28 or 29 at 0.1 or 0.5 mg/kg.

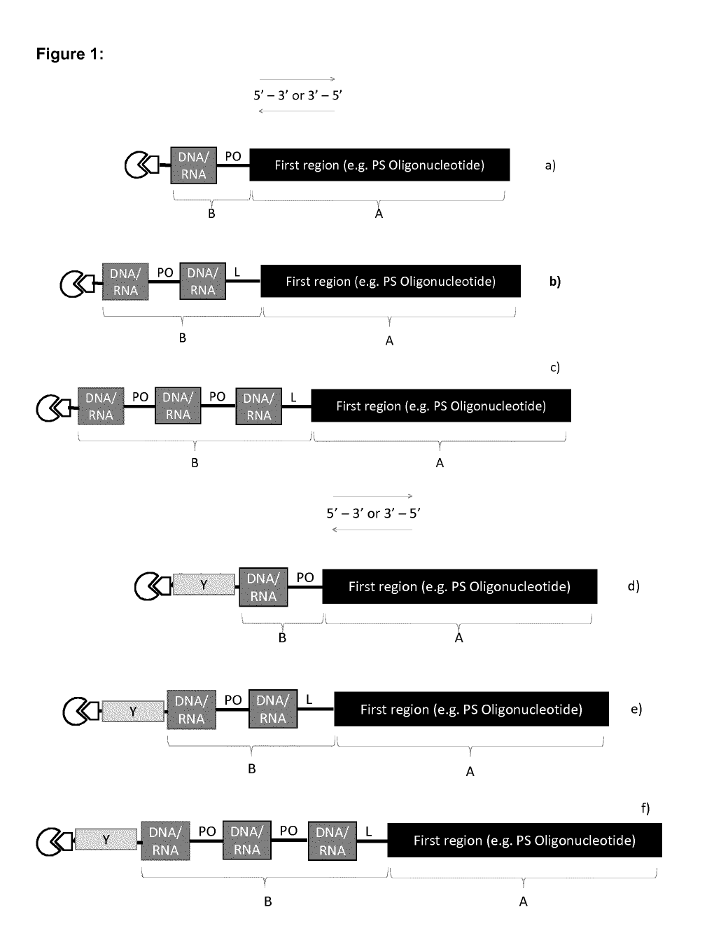

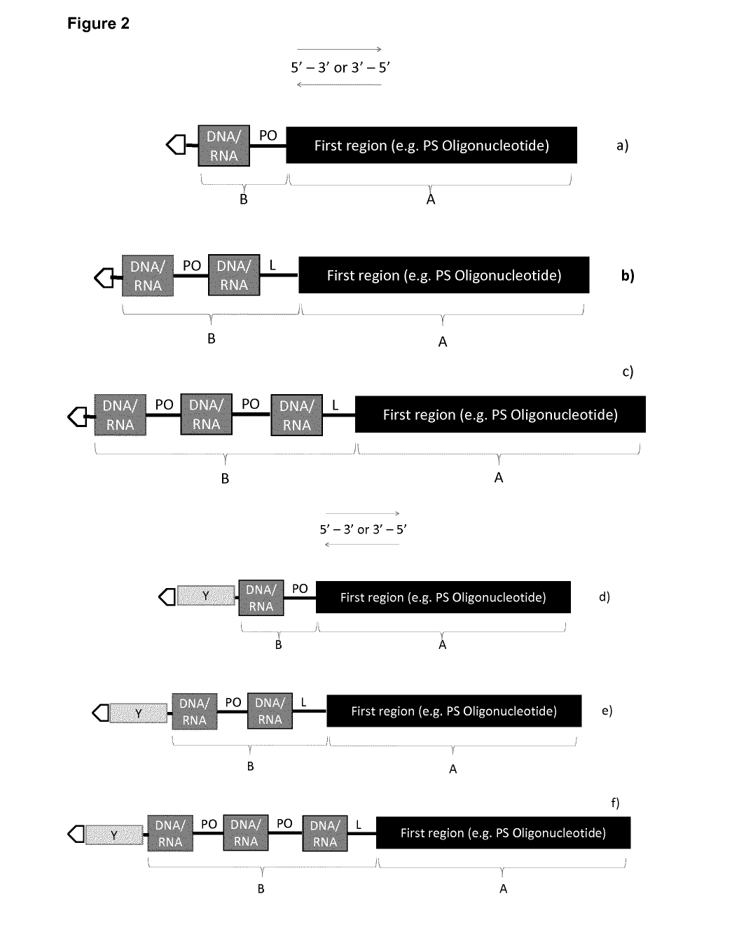

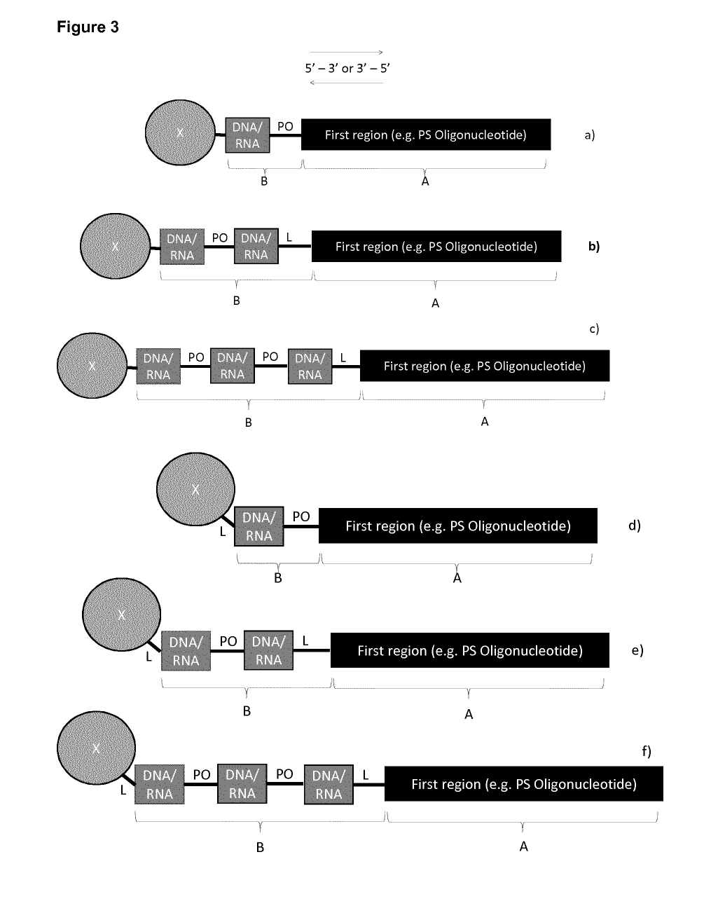

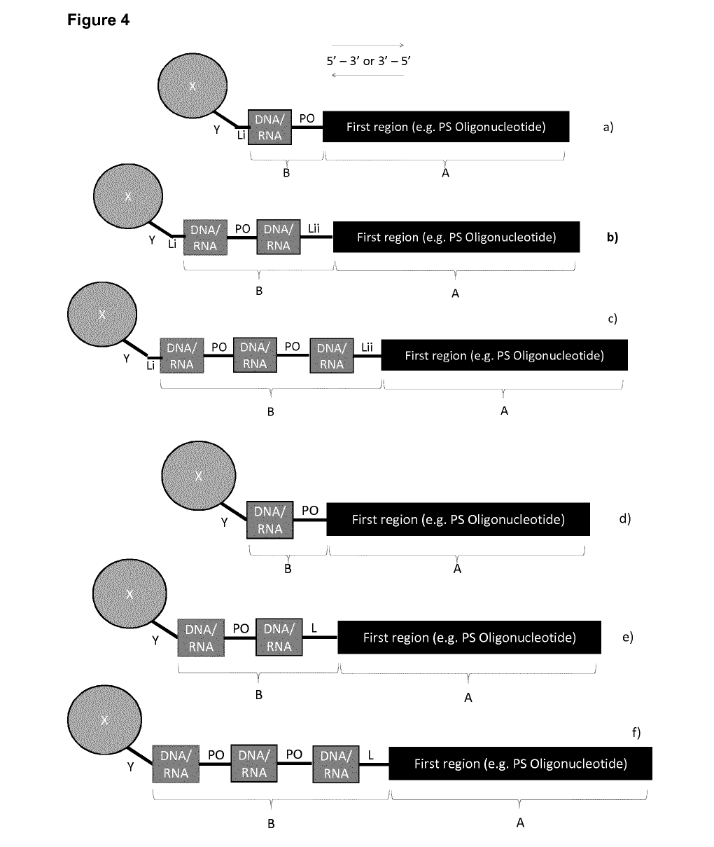

### DETAILED DESCRIPTION OF INVENTION

The antisense oligonucleotide conjugates of the present invention have a number of improved properties over non-conjugated oligonucleotides. The efficacy of the conjugated oligonucleotides is significantly increased. This allows a reduction of dose while still achieving similar effect in terms of reducing ApoB expression and serum cholesterol levels (e.g. improved EC50 and a wider therapeutic index) compared to a corresponding unconjugated compound. Furthermore, some redistribution from the kidney to the liver is observed when the oligonucleotide is conjugated, this may lead to improved safety in addition to the wider therapeutic index achieved by reducing the dose. Finally, the pharmacodynamic half-life of the conjugated oligonucleotides of the invention appear to be significantly longer than for the naked oligonucleotide allowing the effect of the conjugated oligonucleotide to last longer, and thereby potentially reduce the frequency of dosing compared to the naked oligonucleotide.

### The Oligomer

The term “oligomer” or “oligonucleotide” in the context of the present invention, refers to a molecule formed by covalent linkage of two or more nucleotides (i.e. an oligonucleotide). Herein, a single nucleotide (unit) may also be referred to as a monomer or unit. In some embodiments, the terms “nucleoside”, “nucleotide”, “unit” and “monomer” are used interchangeably. It will be recognized that when referring to a sequence of nucleotides or monomers, what is referred to is the sequence of bases, such as A, T, G, C or U.

The invention relates to compounds where an antisense oligonucleotide (oligomer) is joined with a conjugate moiety (Region C), as described in further details in sections below.

An aspect of the invention is an antisense oligonucleotide conjugate comprising an oligomer that targets position 2265 to 2277 on the APOB gene (SEQ ID NO: 32) e.g. an oligomer that is complementary to position 2265 to 2277 on the APOB gene. The aspect includes an antisense oligonucleotide conjugate comprising an oligomer with the oligonucleotide motif of SEQ ID NO 2 or the oligonucleotide sequence of SEQ ID NO 27 joined with a conjugate moiety (region C) comprising a N-acetylgalactosamine moiety or a sterol moiety. Table 1 provides specific combinations of oligomer and conjugates:

TABLE 1
Oligomer/conjugate combinations

| | Conjugate Number (See figures) | | | | | | | | | |
|---|---|---|---|---|---|---|---|---|---|---|
| SEQ ID | Conj1 | Conj2 | Conj3 | Conj4 | Conj1a | Conj2a | Conj3a | Conj4a | Conj5 | Conj6 |
| 2 | C11 | C12 | C13 | C14 | C15 | C16 | C17 | C18 | C19 | C20 |
| 27 | C11 | C12 | C13 | C14 | C15 | C16 | C17 | C18 | C19 | C20 |

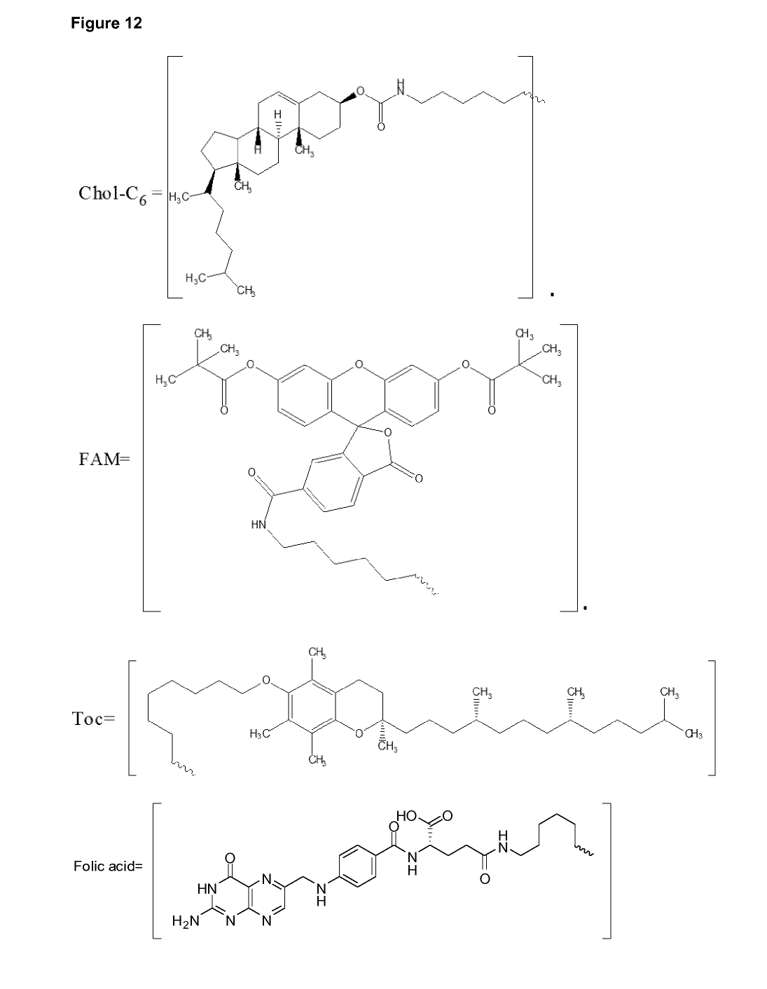

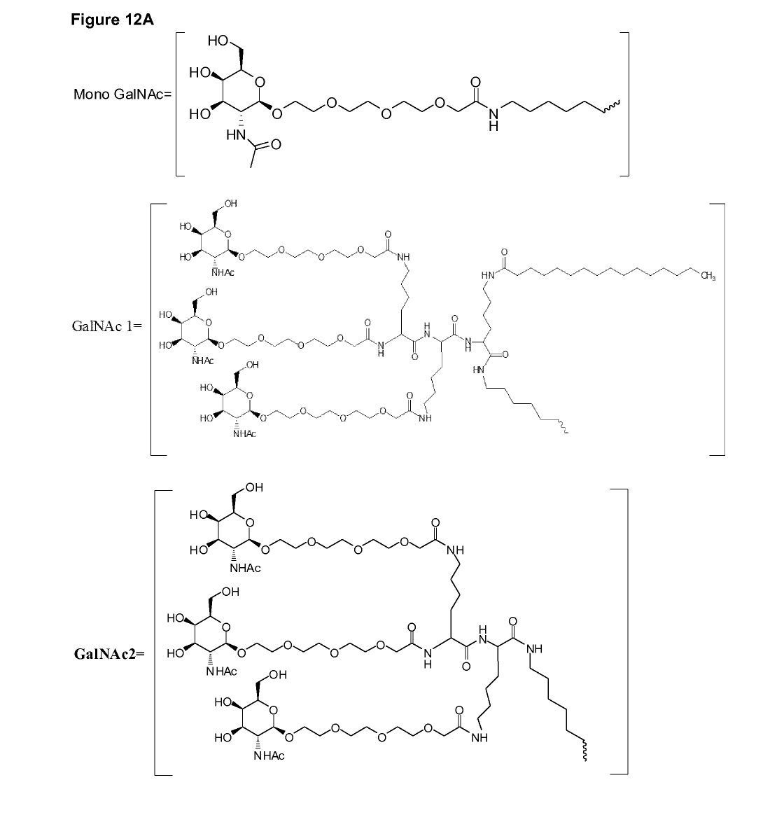

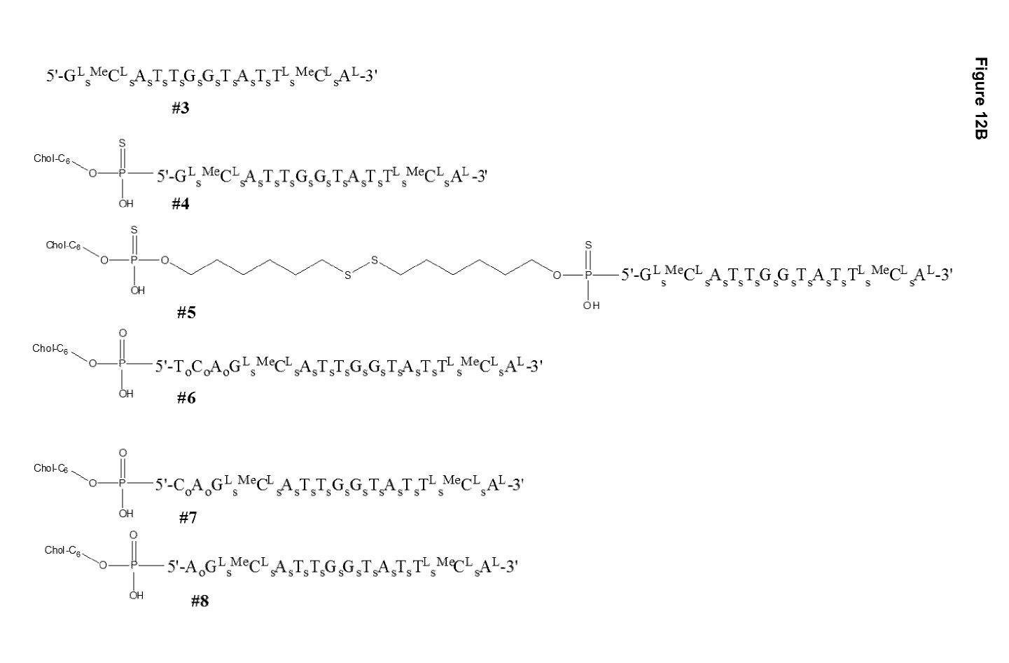

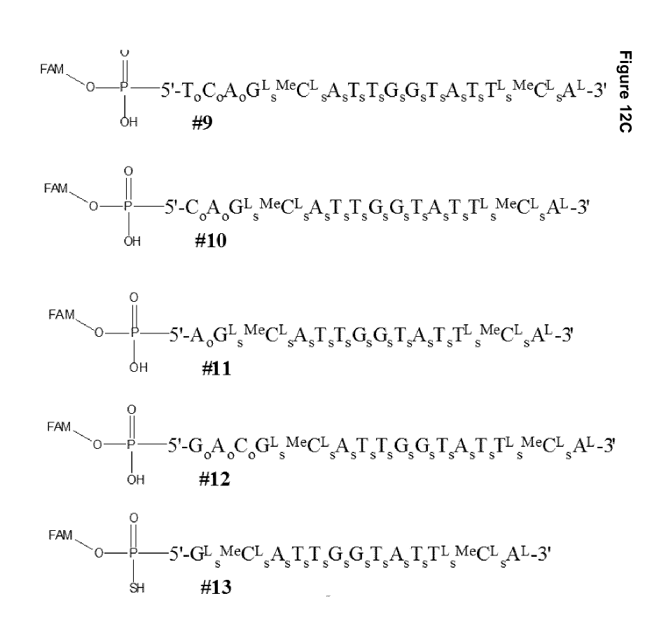

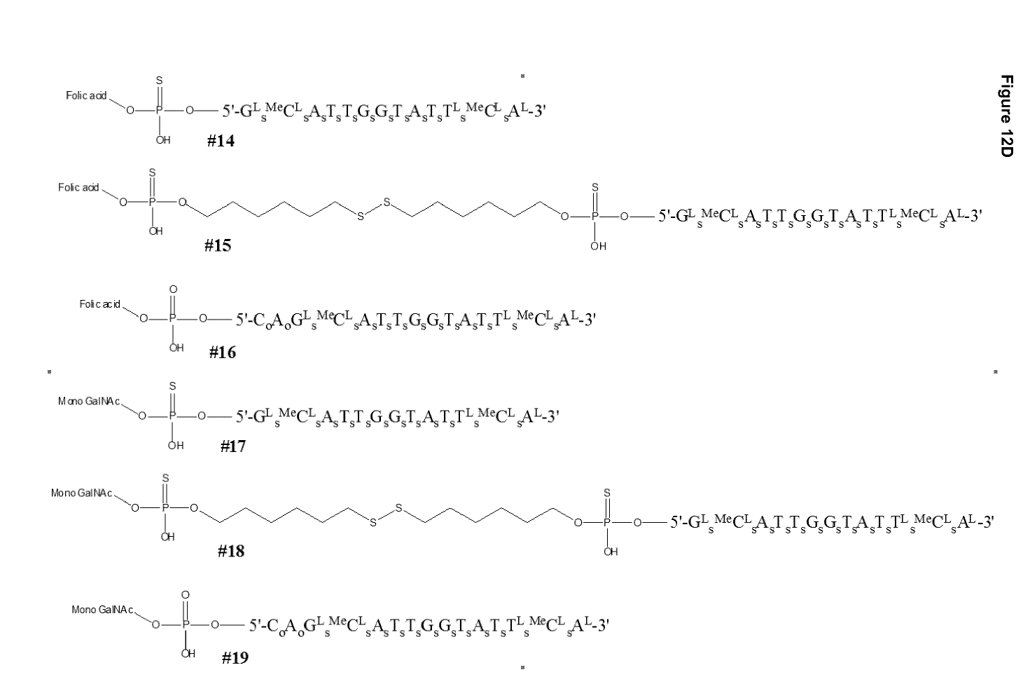

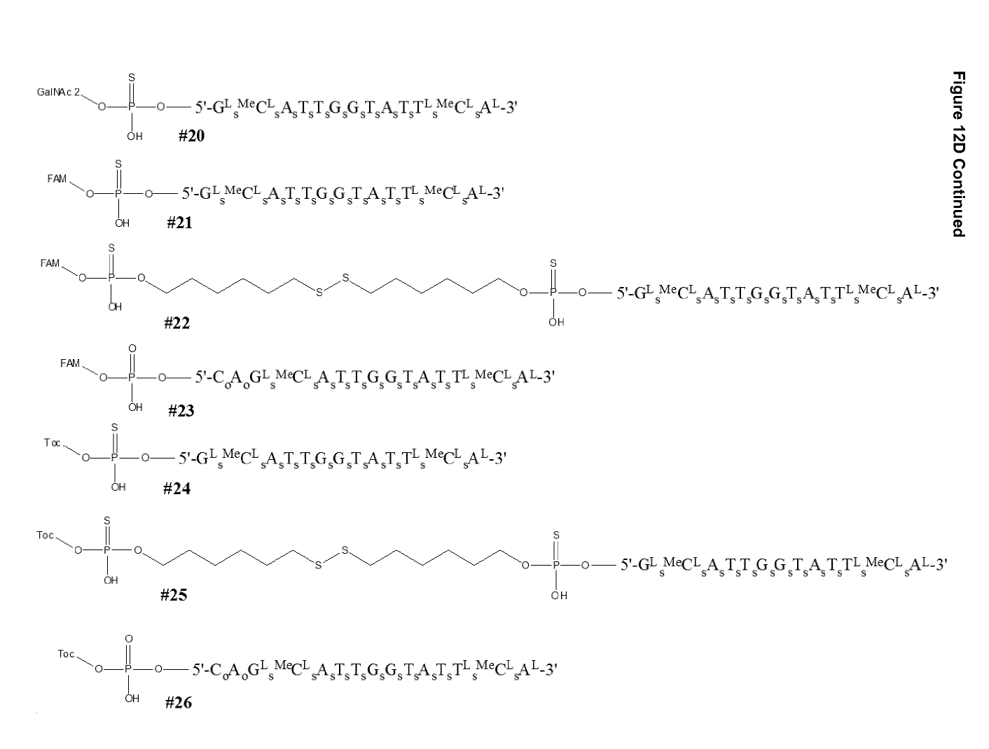

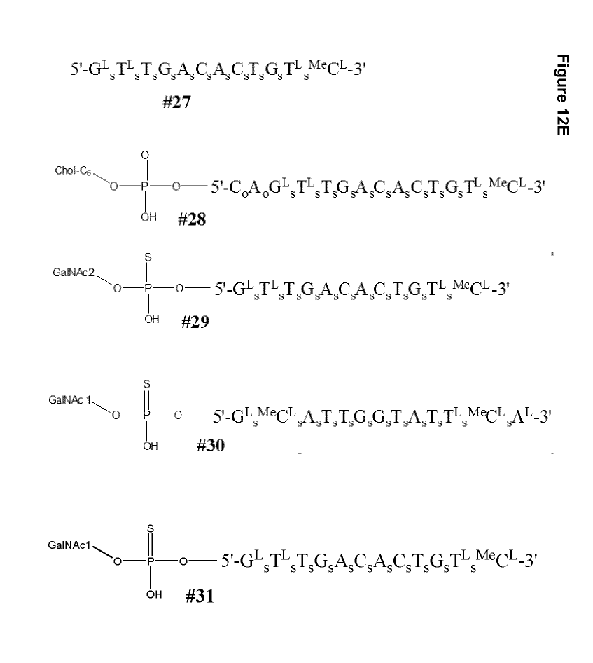

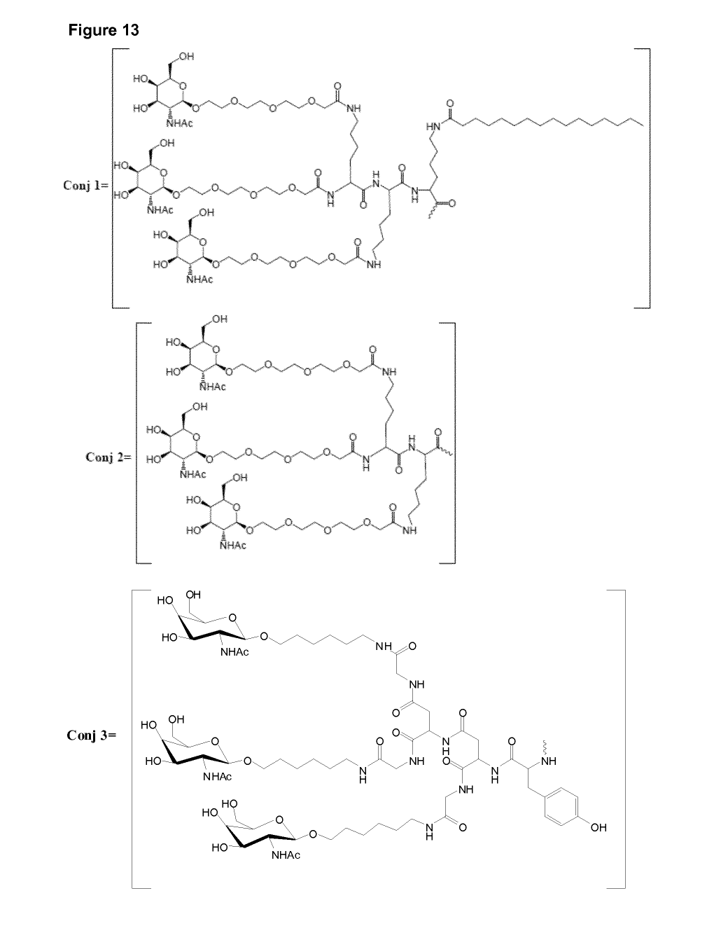

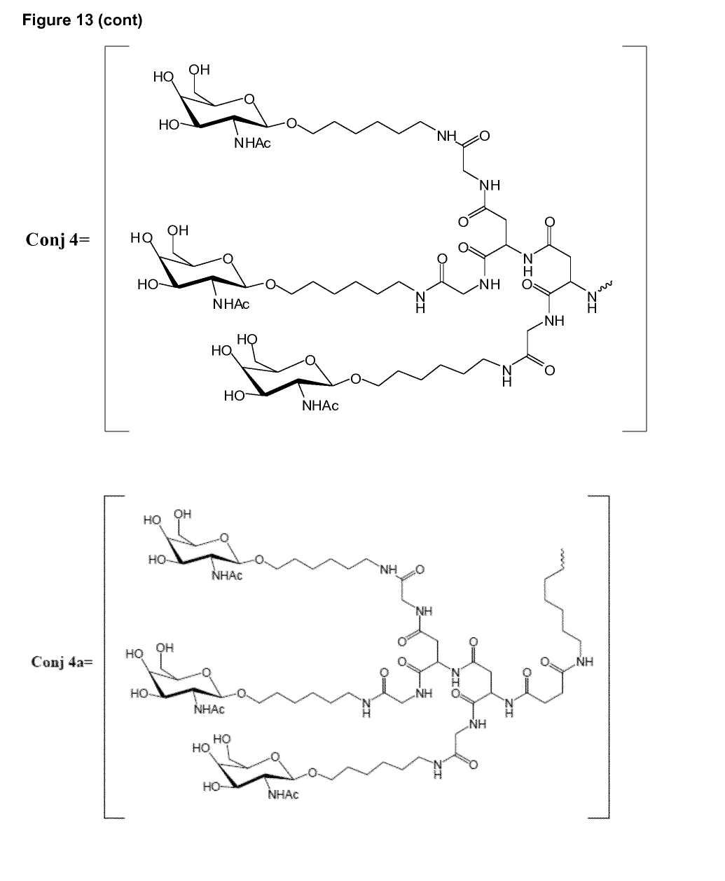

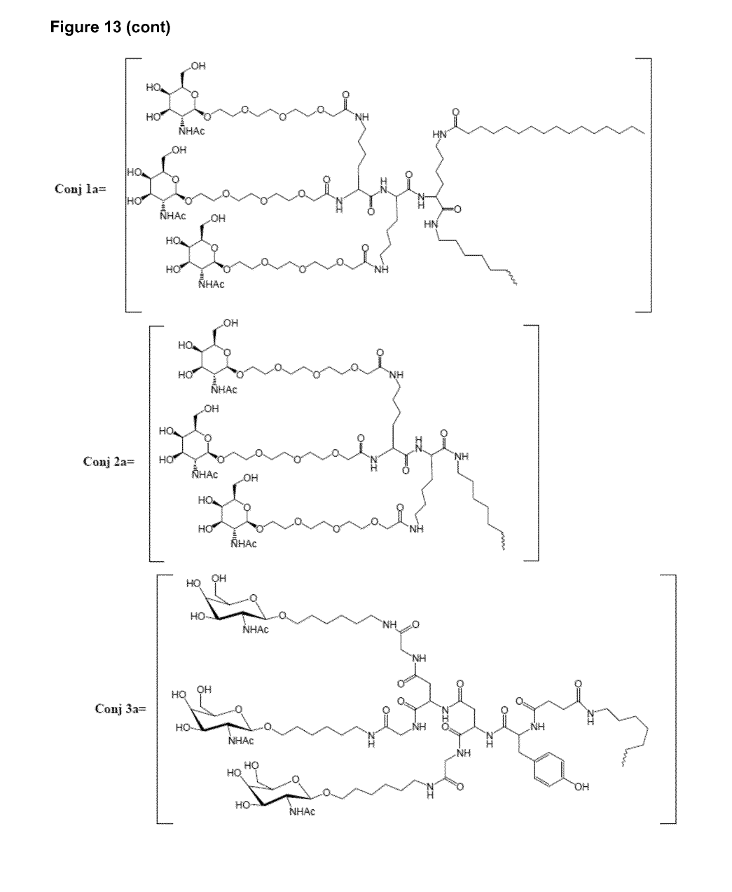

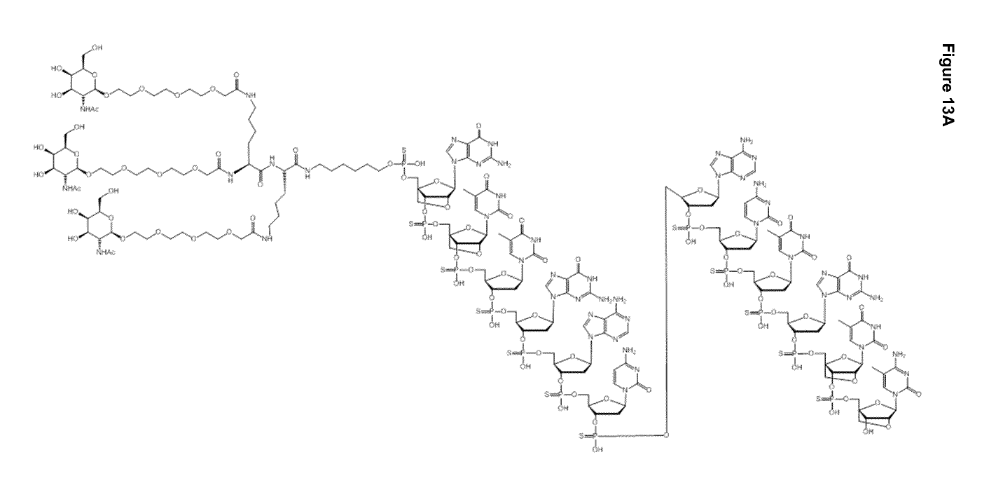

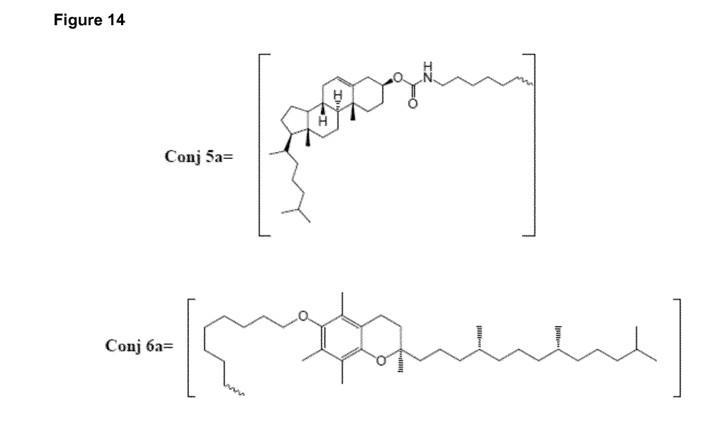

These combinations can be visualized by substituting the wavy line in FIG. 13 or 14 with the sequence of the oligomer. FIG. 12E shows the combination of Conj2 or Conj1 with SEQ ID NO 27, corresponding to SEQ ID NO 31 and 29 respectively. FIG. 13A is a detailed example of the Conj2a compound in FIG. 12E. Please note that a biocleavable linker (B) may or may not be present between the conjugate moiety (C) and the oligomer (A). For Conj1-4 and 1a-4a the GalNAc conjugate itself is biocleavable, utilizing a lysine linker in the GalNAc cluster, and as such a further biocleavable linker (B) may or may not be used. However, preliminary data indicates inclusion of a biocleavable linker (B), such as the phosphate nucleotide linkers disclosed herein may enhance activity of such GalNAc cluster oligomer conjugates. For use with Conj 5 and Conj 6, the use of a biocleavable linker greatly enhances compound activity. Inclusion of a biocleavable linker (B), such as the phosphate nucleotide linkers disclosed herein, is therefore recommended when conjugate moieties comprising sterol is used. The conjugate moiety (and region B or region Y or B and Y, may be positioned, e.g. 5′ or 3′ to the SEQ ID, such as 5′ to region A.

The compound (e.g. oligomer or conjugate) of the invention targets ApoB, and as such is capable of down regulating the APOB expression or reducing ApoB protein levels in an animal, human or in a cell expressing ApoB. In a preferred embodiment the oligonucleotide conjugate of the present invention is capable of reducing the serum ApoB level in an animal or human to a lower level than the unconjugated oligonucleotide with the same sequence when administered at equimolar levels. Preferably, the serum ApoB level is reduced 2 times more by conjugated than by unconjugated oligonucleotide, more preferably 3 times or 4 times more when the oligonucleotide compounds are dosed at, for instance but not limited to, 0.5 mg/kg in a single s.c. injection and measured day 7 after the injection. Even more preferably it is reduced 5 times more by conjugated than by unconjugated oligonucleotide and most preferably it is reduced at least 10 times more by conjugated than by unconjugated oligonucleotide when the oligonucleotide compounds are dosed at, for instance but not limited to, 0.5 mg/kg in a single s.c. injection and measured day 7 after the injection. This allows for a significant reduction in the therapeutic effective amount needed for treatment.

The compound of the invention comprises an oligomer that is between 10-22, such as 10-20, such as 12-22 nucleotides, such as 12-18 nucleotides, such as 13-16 or 12 or 13 or 14 or 15 or 16 nucleotides in length. Details on oligonucleotide length are described in a separate section below.

In some embodiments, the oligomer comprises one or more phosporothiolate linked nucleosides. Details on internucleotide linkages are described in a separate section below.

The compound of the invention comprises an oligonucleotide with the motif of SEQ ID NO 2. In a preferred embodiment the oligonucleotide is a modified oligomer, meaning that it comprises nucleosides or nucleoside linkages that are not naturally occurring. In an embodiment of the invention, the compound of the invention comprises an oligomer with the motif of SEQ ID NO 2, wherein the oligomer comprises or contains at least one nucleotide analogue with a functional effect. The functional effect of the analogue can be producing increased binding to the target and/or increased resistance to intracellular nucleases and/or increased transport into the cell. Details on nucleotide analogue are described in a separate section below. In some embodiments, the nucleotide analogues are sugar modified nucleotides, such as sugar modified nucleotides independently or dependently selected from the group consisting of: Locked Nucleic Acid (LNA) units; 2′-O-alkyl-RNA units, 2′-OMe-RNA units, 2′-amino-DNA units, and 2′-fluoro-DNA units. A preferred nucleotide analogue is LNA.

In some embodiments, the oligomer of the invention comprises or is a gapmer, such as a LNA gapmer oligonucleotide designed based on the motif of SEQ ID NO 2. Details on gapmers and other oligomer designs are described in a separate section below. In preferred embodiments the gapmer corresponds to SEQ ID No 27.

The term “oligonucleotide motif” as used herein describes an oligonucleotide sequence with a defined sequence of bases, such as A, T, G and C that can form the basis for a specific oligonucleotide design where some bases are nucleotide analogues others are DNA or RNA and the linkages can be varied as well.

In a preferred embodiment the oligonucleotide conjugate comprises the LNA oligomer of SEQ ID NO 27, 5′ GTtgacactgTC 3′, wherein the capital letters are LNA nucleosides, and lower case letters are DNA nucleosides, such as the LNA oligomer 5′ GₛTₛtₛgₛaₛcₛaₛcₛtₛgₛTₛC 3′ (region A), wherein capital letters represent beta-D-oxy LNA, lower case letters represent DNA nucleosides, LNA cytosines are 5-methyl cytosine, and all internucleoside linkages are phosphorothioate.

The compound of the invention may comprise a further nucleotide region. In some embodiments, the further nucleotide region comprises a biocleavable nucleotide region, such as a phosphate nucleotide sequence (a second region, region B), which may covalently link region A to a non-nucleotide moiety, such as a conjugate group, (a third region, or region C). In some embodiments the contiguous nucleotide sequence of the oligomer of the invention (region A) is directly covalently linked to region C. In some embodiments region C is biocleavable. More details on linkers are found in the sections below.

In various embodiments, the compound of the invention does not comprise RNA (units). In some embodiments, the compound according to the invention, the first region, or the first and second regions together (e.g. as a single contiguous sequence), is a linear molecule or is synthesised as a linear molecule. The oligomer may therefore be single stranded molecule. In some embodiments, the oligomer does not comprise short regions of, for example, at least 3, 4 or 5 contiguous nucleotides, which are complementary to equivalent regions within the same oligomer (i.e. the oligo does not form duplexes). The oligomer, in some embodiments, may be not (essentially) double stranded. In some embodiments, the oligomer is essentially not double stranded, such as is not a siRNA.

### The Target

Suitably the oligomer of the invention is capable of down-regulating expression of the APO-B gene, such as ApoB-100 or ApoB-48 (APOB). In this regards, the oligomer of the invention can affect the inhibition of APOB, typically in a mammalian such as a human cell, such as liver cells. In some embodiments, the oligomers of the invention bind to the target nucleic acid and effect inhibition of expression of at least 10% or 20% compared to the normal expression level, more preferably at least a 30%, 40%, 50%, 60%, 70%, 80%, 90% or 95% inhibition compared to the normal expression level. In some embodiments, such modulation is seen when using between 0.04 and 25 nM, such as between 0.8 and 20 nM concentration of the compound of the invention. In the same or a different embodiment, the inhibition of expression is less than 100%, such as less than 98% inhibition, less than 95% inhibition, less than 90% inhibition, less than 80% inhibition, such as less than 70% inhibition. Modulation of expression level may be determined by measuring protein levels, e.g. by the methods such as SDS-PAGE followed by western blotting using suitable antibodies raised against the target protein. Alternatively, modulation of expression levels can be determined by measuring levels of mRNA, e.g. by northern blotting or quantitative RT-PCR. When measuring via mRNA levels, the level of down-regulation when using an appropriate dosage, such as between 0.04 and 25 nM, such as between 0.8 and 20 nM concentration, is, in some embodiments, typically to a level of between 10-20% the normal levels in the absence of the compound of the invention.

The invention therefore provides a method of down-regulating or inhibiting the expression of APO-B protein and/or mRNA in a cell which is expressing APO-B protein and/or mRNA, said method comprising administering the compound of the invention to the invention to said cell to down-regulating or inhibiting the expression of APO-B protein and/or mRNA in said cell. Suitably the cell is a mammalian cell such as a human cell. The administration may occur, in some embodiments, in vitro. The administration may occur, in some embodiments, in vivo.

The term “target nucleic acid”, as used herein refers to the DNA or RNA encoding mammalian APO-B polypeptide, such as human APO-B100, such as human APO-B100 mRNA. APO-B100 encoding nucleic acids or naturally occurring variants thereof, and RNA nucleic acids derived therefrom, preferably mRNA, such as pre-mRNA, although preferably mature mRNA. An example of the target ApoB nucleic acid is given in SEQ ID No 32 corresponding to NCBI accession No NM_000384. Target ApoB nucleic acids are also found as genbank accession No: NG_011793, NM_000384.2, GI:105990531 and NG_011793.1 GI:226442987, all are hereby incorporated by reference. In some embodiments, for example when used in research or diagnostics the “target nucleic acid” may be a cDNA or a synthetic oligonucleotide derived from the above DNA or RNA nucleic acid targets. The oligomer according to the invention is preferably capable of hybridising to the target nucleic acid. It will be recognised that human APO-B mRNA is a cDNA sequence, and as such, corresponds to the mature mRNA target sequence, although uracil is replaced with thymidine in the cDNA sequences.

The term “naturally occurring variant thereof” refers to variants of the APO-B1 polypeptide of nucleic acid sequence which exist naturally within the defined taxonomic group, such as mammalian, such as mouse, monkey, and preferably human. Typically, when referring to “naturally occurring variants” of a polynucleotide the term also may encompass any allelic variant of the APO-B encoding genomic DNA by chromosomal translocation or duplication, and the RNA, such as mRNA derived therefrom. “Naturally occurring variants” may also include variants derived from alternative splicing of the APO-B100 mRNA. When referenced to a specific polypeptide sequence, e.g., the term also includes naturally occurring forms of the protein which may therefore be processed, e.g. by co- or post-translational modifications, such as signal peptide cleavage, proteolytic cleavage, glycosylation, etc.

The oligomers (region A) comprise or consist of a contiguous nucleotide sequence which corresponds to the reverse complement of a nucleotide sequence present in e.g. the human APO-B mRNA.

The terms “corresponding to” and “corresponds to” refer to the comparison between the nucleotide sequence of the oligomer (i.e. the nucleobase or base sequence) or contiguous nucleotide sequence (a first region/region A) and the reverse complement of the nucleic acid target, or sub-region thereof.

Nucleotide analogues are compared directly to their equivalent or corresponding nucleotides. In a preferred embodiment, the oligomers (or first region thereof) are complementary to the target region or sub-region, such as fully complementary.

The terms “reverse complement”, “reverse complementary” and “reverse complementarity” as used herein are interchangeable with the terms “complement”, “complementary” and “complementarity”.

The terms “corresponding nucleotide analogue” and “corresponding nucleotide” are intended to indicate that the nucleotide in the nucleotide analogue and the naturally occurring nucleotide are identical. For example, when the 2-deoxyribose unit of the nucleotide is linked to an adenine, the “corresponding nucleotide analogue” contains a pentose unit (different from 2-deoxyribose) linked to an adenine.

The term “nucleobase” refers to the base moiety of a nucleotide and covers both naturally occurring a well as non-naturally occurring variants. Thus, “nucleobase” covers not only the known purine and pyrimidine heterocycles but also heterocyclic analogues and tautomeres thereof. It will be recognized that the DNA or RNA nucleosides of region B may have a naturally occurring and/or non-naturally occurring nucleobase(s).

Examples of nucleobases include, but are not limited to adenine, guanine, cytosine, thymidine, uracil, xanthine, hypoxanthine, 5-methylcytosine, isocytosine, pseudoisocytosine, 5-bromouracil, 5-propynyluracil, 6-aminopurine, 2-aminopurine, inosine, diaminopurine, and 2-chloro-6-aminopurine. In some embodiments the nucleobases may be independently selected from the group consisting of adenine, guanine, cytosine, thymidine, uracil, and 5-methylcytosine. In some embodiments the nucleobases may be independently selected from the group consisting of adenine, guanine, cytosine, thymidine, and 5-methylcytosine.

In some embodiments, at least one of the nucleobases present in the oligomer is a modified nucleobase selected from the group consisting of 5-methylcytosine, isocytosine, pseudoisocytosine, 5-bromouracil, 5-propynyluracil, 6-aminopurine, 2-aminopurine, inosine, diaminopurine, and 2-chloro-6-aminopurine.

### Nucleotide Analogues

The term “nucleotide” as used herein, refers to a glycoside comprising a sugar moiety, a base moiety and a covalently linked group, such as a phosphate or phosphorothioate internucleotide linkage group, and covers both naturally occurring nucleotides, such as DNA or RNA, and non-naturally occurring nucleotides comprising modified sugar and/or base moieties, which are also referred to as “nucleotide analogues” herein. Herein, a single nucleotide (unit) may also be referred to as a monomer or nucleic acid unit.

In field of biochemistry, the term “nucleoside” is commonly used to refer to a glycoside comprising a sugar moiety and a base moiety, and may therefore be used when referring to the nucleotide units, which are covalently linked by the internucleotide linkages between the nucleotides of the oligomer.

As one of ordinary skill in the art would recognise, the 5′ nucleotide of an oligonucleotide does not comprise a 5′ internucleotide linkage group, although may or may not comprise a 5′ terminal group.

Non-naturally occurring nucleotides include nucleotides which have modified sugar moieties, such as bicyclic nucleotides or 2′ modified nucleotides, such as 2′ substituted nucleotides.

“Nucleotide analogues” are variants of natural nucleotides, such as DNA or RNA nucleotides, by virtue of modifications in the sugar and/or base moieties. Analogues could in principle be merely “silent” or “equivalent” to the natural nucleotides in the context of the oligonucleotide, i.e. have no functional effect on the way the oligonucleotide works to inhibit target gene expression. Such “equivalent” analogues may nevertheless be useful if, for example, they are easier or cheaper to manufacture, or are more stable to storage or manufacturing conditions, or represent a tag or label. Preferably, however, the analogues will have a functional effect on the way in which the oligomer works to inhibit expression; for example by producing increased binding affinity to the target and/or increased resistance to intracellular nucleases and/or increased ease of transport into the cell. Specific examples of nucleoside analogues are described by e.g. Freier & Altmann; Nucl. Acid Res., 1997, 25, 4429-4443 and Uhlmann; Curr. Opinion in Drug Development, 2000, 3(2), 293-213, and in Scheme 1:

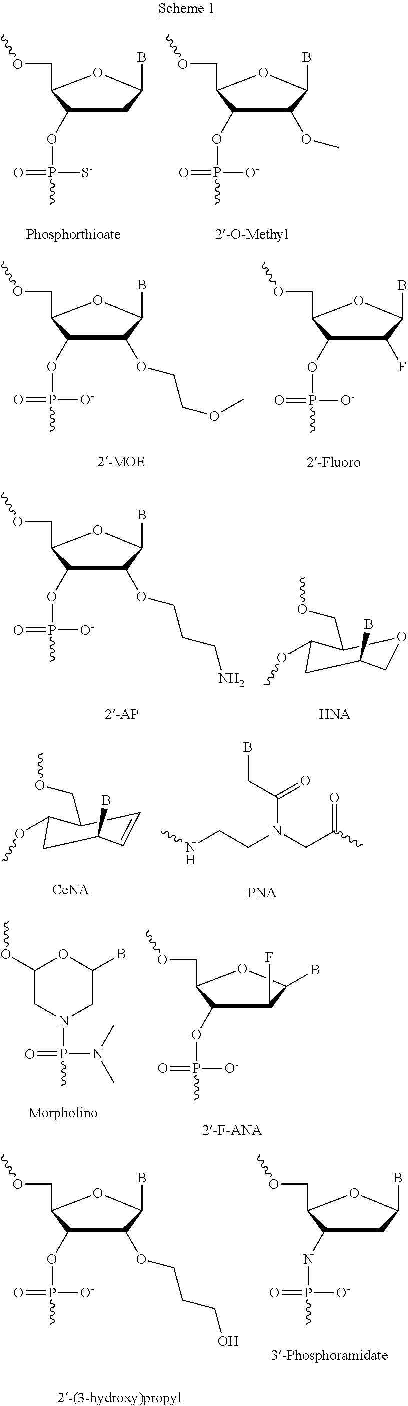

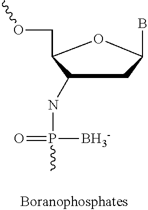

The oligomer may thus comprise or consist of a simple sequence of natural occurring nucleotides—preferably 2′-deoxynucleotides (referred here generally as “DNA”), but also possibly ribonucleotides (referred here generally as “RNA”), or a combination of such naturally occurring nucleotides and one or more non-naturally occurring nucleotides, i.e. nucleotide analogues. Such nucleotide analogues may suitably enhance the affinity of the oligomer for the target sequence.

As used herein, “2′-modified” or “2′-substituted” refers to a nucleoside comprising a sugar comprising a substituent at the 2′ position other than H or OH. 2′-modified nucleosides, include, but are not limited to nucleosides with non-bridging 2′substituents, such as allyl, amino, azido, thio, O-allyl, O—C₁-C₁₀ alkyl, —OCF₃, O—(CH₂)₂—O—CH₃, 2′-O(CH₂)₂SCH₃, O—(CH₂)₂—O—N(Rₘ)(Rₙ), or O—CH₂—C(═O)—N(Rₘ)(Rₙ), where each Rₙ, and R is, independently, H or substituted or unsubstituted C₁-C₁₀ alkyl. 2′-modified nucleosides may further comprise other modifications, for example, at other positions of the sugar and/or at the nucleobase.

As used herein, “2′-F” refers to a sugar comprising a fluoro group at the 2′ position.

As used herein, “2′-OMe” or “2′-OCH₃” or “2′-O-methyl” each refers to a nucleoside

Examples of suitable and preferred nucleotide analogues are provided by WO2007/031091 or are referenced therein. Other nucleotide analogues which may be used in the oligomer of the invention include tricyclic nucleic acids, for example please see WO2013154798 and WO2013154798 which are hereby incorporated by reference.

Incorporation of affinity-enhancing nucleotide analogues in the oligomer, such as LNA or 2′-substituted sugars, can allow the size of the specifically binding oligomer to be reduced, and may also reduce the upper limit to the size of the oligomer before non-specific or aberrant binding takes place.

In some embodiments of the invention nucleotide analogues present within the oligomer of the invention are independently selected from, for example: 2′-O-alkyl-RNA units, 2′-amino-DNA units, 2′-fluoro-DNA units, LNA units, arabino nucleic acid (ANA) units, 2′-fluoro-ANA units, HNA units, INA (intercalating nucleic acid—Christensen, 2002. Nucl. Acids. Res. 2002 30: 4918-4925, hereby incorporated by reference) units and 2′MOE units. In some embodiments there is only one of the above types of nucleotide analogues present in the oligomer of the invention, such as the first region, or contiguous nucleotide sequence thereof.

In some embodiments the oligomer comprises at least 2 nucleotide analogues. In some embodiments, the oligomer comprises from 3-8 nucleotide analogues, e.g. 6 or 7 nucleotide analogues. In the by far most preferred embodiments, at least one of said nucleotide analogues is a locked nucleic acid (LNA); for example at least 3 or at least 4, or at least 5, or at least 6, or at least 7, or 8, of the nucleotide analogues may be LNA. In some embodiments all the nucleotides analogues may be LNA. LNA analogues are described in more detail in a separate section.

It will be recognised that when referring to a preferred nucleotide sequence motif or nucleotide sequence, which consists of only nucleotides, the oligomers of the invention which are defined by that sequence may comprise a corresponding nucleotide analogue in place of one or more of the nucleotides present in said sequence, such as LNA units or other nucleotide analogues, which raise the duplex stability/Tₘ of the oligomer/target duplex (i.e. affinity enhancing nucleotide analogues).

Tₘ Assay: The oligonucleotide: Oligonucleotide and RNA target (PO) duplexes are diluted to 3 mM in 500 ml RNase-free water and mixed with 500 ml 2×Tₘ-buffer (200 mM NaCl, 0.2 mM EDTA, 20 mM Naphosphate, pH 7.0). The solution is heated to 95° C. for 3 min and then allowed to anneal in room temperature for 30 min. The duplex melting temperatures (Tₘ) is measured on a Lambda 40 UV/VIS Spectrophotometer equipped with a Peltier temperature programmer PTP6 using PE Templab software (Perkin Elmer). The temperature is ramped up from 20° C. to 95° C. and then down to 25° C., recording absorption at 260 nm. First derivative and the local maximums of both the melting and annealing are used to assess the duplex Tₘ.

### LNA

The term “LNA” refers to a bicyclic nucleoside analogue which comprises a C2*-C4* biradical (a bridge), and is known as “Locked Nucleic Acid”. It may refer to an LNA monomer, or, when used in the context of an “LNA oligonucleotide”, LNA refers to an oligonucleotide containing one or more such bicyclic nucleotide analogues. In some aspects bicyclic nucleoside analogues are LNA nucleotides, and these terms may therefore be used interchangeably, and is such embodiments, both are be characterized by the presence of a linker group (such as a bridge) between C2′ and C4′ of the ribose sugar ring.

In some embodiments, at least one nucleoside analogue present in the first region (A) is a bicyclic nucleoside analogue, such as at least 2, at least 3, at least 4, at least 5, at least 6, at least 7, at least 8, (except the DNA and or RNA nucleosides of region B) are sugar modified nucleoside analogues, such as such as bicyclic nucleoside analogues, such as LNA, e.g. beta-D-X-LNA or alpha-L-X-LNA (wherein X is oxy, amino or thio), or other LNAs disclosed herein including, but not limited to ENA, (R/S) cET, cMOE or 5′-Me-LNA.

LNA used in the oligonucleotide compounds of the invention preferably has the structure of the two exemplary stereochemical isomers shown below which include the beta-D and alpha-L isoforms:

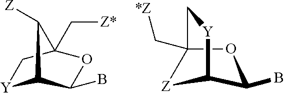

Specific exemplary LNA units are shown below:

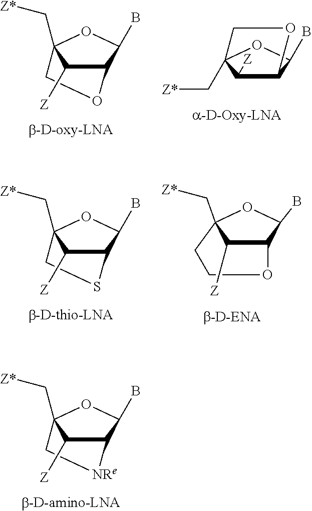

A preferred nucleotide analogue is LNA, such as oxy-LNA (such as beta-D-oxy-LNA, and alpha-L-oxy-LNA), and/or amino-LNA (such as beta-D-amino-LNA and alpha-L-amino-LNA) and/or thio-LNA (such as beta-D-thio-LNA and alpha-L-thio-LNA) and/or ENA (such as beta-D-ENA and alpha-L-ENA). In preferred embodiments LNA is beta-D-oxy-LNA.

The term “thio-LNA” comprises a locked nucleotide in which O in the general formula above is selected from S or —CH₂—S—. Thio-LNA can be in both beta-D and alpha-L-configuration.

The term “amino-LNA” comprises a locked nucleotide in which Y in the general formula above is selected from —N(H)—, N(R)—, CH₂—N(H)—, and —CH₂—N(R)— where R is selected from hydrogen and C₁₋₄-alkyl. Amino-LNA can be in both beta-D and alpha-L-configuration.

The term “oxy-LNA” comprises a locked nucleotide in which Y in the general formula above represents —O—. This can also be described as 2′-O—(CH₂)-4′ or 4′-(CH₂)—O-2′. Oxy-LNA can be in both beta-D and alpha-L-configuration.

The term “ENA” comprises a locked nucleotide in which Y in the general formula above is —CH₂—O— (where the oxygen atom of —CH₂—O— is attached to the 2′-position relative to the base B). This can also be described as 2′-O—(CH₂)₂-4′ or 4′-(CH₂)₂—O-2′

Other LNA nucleosides which may be used in place of beta-D-oxy LNA are provided in PCT/EP2013/073858, hereby incorporated by reference, for example.

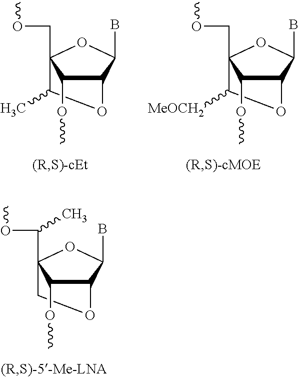

Incorporation of affinity-enhancing nucleotide analogues in the oligomer, such as LNA or 2′-substituted sugars, can allow the size of the specifically binding oligomer to be reduced, and may also reduce the upper limit to the size of the oligomer before non-specific or aberrant binding takes place.

### Length

The oligomers may comprise or consist of a contiguous nucleotide sequence of a total of between 10, 11, 12, 13, 14, 15, 16, 17, 18, 19, 20, 21, or 22 contiguous nucleotides in length. Lengths may include region A or region A and B for example.

In some embodiments, the oligomers comprise or consist of a contiguous nucleotide sequence of a total of between 10-22, such as 12-18, such as 13-17 or 13-16 or 12-16 or 12-14, such as 12, 13, 14, 15, 16 contiguous nucleotides in length.

In some embodiments, the oligomer according to the invention consists of no more than 22 nucleotides, such as no more than 20 nucleotides, such as no more than 18 nucleotides, such as 15, 16 or 17 nucleotides. In some embodiments the oligomer of the invention comprises less than 20 nucleotides.

### RNAse Recruitment

It is recognised that an oligomeric compound may function via non RNase mediated degradation of target mRNA, such as by steric hindrance of translation, or other methods, In some embodiments, the oligomers of the invention are capable of recruiting an endoribonuclease (RNase), such as RNase H.

It is preferable such oligomers, such as region A, or contiguous nucleotide sequence, comprises of a region of at least 6, such as at least 7 consecutive nucleotide units, such as at least 8 or at least 9 consecutive nucleotide units (residues), including 7, 8, 9, 10, 11, 12, 13, 14, 15 or 16 consecutive nucleotides, which, when formed in a duplex with the complementary target RNA is capable of recruiting RNase (such as DNA units). The contiguous sequence which is capable of recruiting RNAse may be region Y′ as referred to in the context of a gapmer as described herein. In some embodiments the size of the contiguous sequence which is capable of recruiting RNAse, such as region Y′, may be higher, such as 10, 11, 12, 13, 14, 15, 16, 17, 18, 19 or 20 nucleotide units.

EP 1 222 309 provides in vitro methods for determining RNaseH activity, which may be used to determine the ability to recruit RNaseH. A oligomer is deemed capable of recruiting RNase H if, when provided with the complementary RNA target, it has an initial rate, as measured in pmol/l/min, of at least 1%, such as at least 5%, such as at least 10% or, more than 20% of the of the initial rate determined using DNA only oligonucleotide, having the same base sequence but containing only DNA monomers, with no 2′ substitutions, with phosphorothioate linkage groups between all monomers in the oligonucleotide, using the methodology provided by Example 91-95 of EP 1 222 309.

In some embodiments, an oligomer is deemed essentially incapable of recruiting RNaseH if, when provided with the complementary RNA target, and RNaseH, the RNaseH initial rate, as measured in pmol/l/min, is less than 1%, such as less than 5%, such as less than 10% or less than 20% of the initial rate determined using the equivalent DNA only oligonucleotide, with no 2′ substitutions, with phosphorothioate linkage groups between all nucleotides in the oligonucleotide, using the methodology provided by Example 91-95 of EP 1 222 309.

In other embodiments, an oligomer is deemed capable of recruiting RNaseH if, when provided with the complementary RNA target, and RNaseH, the RNaseH initial rate, as measured in pmol/l/min, is at least 20%, such as at least 40%, such as at least 60%, such as at least 80% of the initial rate determined using the equivalent DNA only oligonucleotide, with no 2′ substitutions, with phosphorothioate linkage groups between all nucleotides in the oligonucleotide, using the methodology provided by Example 91-95 of EP 1 222 309.

Typically the region of the oligomer which forms the consecutive nucleotide units which, when formed in a duplex with the complementary target RNA is capable of recruiting RNase consists of nucleotide units which form a DNA/RNA like duplex with the RNA target. The oligomer of the invention, such as the first region, may comprise a nucleotide sequence which comprises both nucleotides and nucleotide analogues, and may be e.g. in the form of a gapmer.

### Gapmer Design

In some embodiments, the oligomer of the invention, such as the first region, comprises or is a gapmer. A gapmer oligomer is an oligomer which comprises a contiguous stretch of nucleotides which is capable of recruiting an RNAse, such as RNAseH, such as a region of at least 6 or 7 DNA nucleotides, referred to herein in as region Y′ (Y′), wherein region Y′ is flanked both 5′ and 3′ by regions of affinity enhancing nucleotide analogues, such as from 1-6 nucleotide analogues 5′ and 3′ to the contiguous stretch of nucleotides which is capable of recruiting RNAse—these regions are referred to as regions X′ (X′) and Z′ (Z′) respectively. The X′ and Z′ regions can also be termed the wings of the gapmer and region Y′ is also termed the gap of the gapmer. Examples of gapmers are disclosed in WO2004/046160, WO2008/113832, and WO2007/146511.

In some embodiments, the monomers which are capable of recruiting RNAse are selected from the group consisting of DNA monomers, alpha-L-LNA monomers, C4′ alkylayted DNA monomers (see PCT/EP2009/050349 and Vester et al., Bioorg. Med. Chem. Lett. 18 (2008) 2296-2300, hereby incorporated by reference), and UNA (unlinked nucleic acid) nucleotides (see Fluiter et al., Mol. Biosyst., 2009, 10, 1039 hereby incorporated by reference). UNA is unlocked nucleic acid, typically where the C2-C3 C—C bond of the ribose has been removed, forming an unlocked “sugar” residue. Preferably the gapmer comprises a (poly)nucleotide sequence of formula (5′ to 3′), X′-Y′-Z′, wherein; region X′ (X′) (5′ region) consists or comprises of at least one nucleotide analogue, such as at least one LNA unit, such as from 1-6 nucleotide analogues, such as LNA units, and; region Y′ (Y′) consists or comprises of at least five consecutive nucleotides which are capable of recruiting RNAse (when formed in a duplex with a complementary RNA molecule, such as the mRNA target), such as DNA nucleotides, and; region Z′ (Z′) (3′region) consists or comprises of at least one nucleotide analogue, such as at least one LNA unit, such as from 1-6 nucleotide analogues, such as LNA units.

In some embodiments, region X′ consists of 1, 2, 3, 4, 5 or 6 nucleotide analogues, such as LNA units, such as from 2-5 nucleotide analogues, such as 2-5 LNA units, such as 3 or 4 nucleotide analogues, such as 3 or 4 LNA units; and/or region Z′ consists of 1, 2, 3, 4, 5 or 6 nucleotide analogues, such as LNA units, such as from 2-5 nucleotide analogues, such as 2-5 LNA units, such as 3 or 4 nucleotide analogues, such as 3 or 4 LNA) units.

In some embodiments Y′ consists or comprises of 5, 6, 7, 8, 9, 10, 11 or 12 consecutive nucleotides which are capable of recruiting RNAse, or from 6-10, or from 7-9, such as 8 consecutive nucleotides which are capable of recruiting RNAse. In some embodiments region Y′ consists or comprises at least one DNA nucleotide unit, such as 1-12 DNA units, preferably from 4-12 DNA units, more preferably from 6-10 DNA units, such as from 7-10 DNA units, most preferably 8, 9 or 10 DNA units.

In some embodiments region X′ consist of 3 or 4 nucleotide analogues, such as LNA, region X′ consists of 7, 8, 9 or 10 DNA units, and region Z′ consists of 3 or 4 nucleotide analogues, such as LNA. Such designs include (X′-Y′-Z′) 3-10-3, 3-10-4, 4-10-3, 3-9-3, 3-9-4, 4-9-3, 3-8-3, 3-8-4, 4-8-3, 3-7-3, 3-7-4, 4-7-3.

Further gapmer designs are disclosed in WO2004/046160, which is hereby incorporated by reference. WO2008/113832, which claims priority from U.S. provisional application 60/977,409 hereby incorporated by reference, refers to ‘shortmer’ gapmer oligomers. In some embodiments, oligomers presented here may be such shortmer gapmers.

In some embodiments the oligomer, e.g. region X′, is consisting of a contiguous nucleotide sequence of a total of 10, 11, 12, 13 or 14 nucleotide units, wherein the contiguous nucleotide sequence comprises or is of formula (5′-3′), X′-Y′-Z′ wherein; X′ consists of 1, 2 or 3 nucleotide analogue units, such as LNA units; Y′ consists of 7, 8 or 9 contiguous nucleotide units which are capable of recruiting RNAse when formed in a duplex with a complementary RNA molecule (such as a mRNA target); and Z′ consists of 1, 2 or 3 nucleotide analogue units, such as LNA units.

In some embodiments X′ consists of 1 LNA unit. In some embodiments X′ consists of 2 LNA units. In some embodiments X′ consists of 3 LNA units. In some embodiments Z′ consists of 1 LNA units. In some embodiments Z′ consists of 2 LNA units. In some embodiments Z′ consists of 3 LNA units. In some embodiments Y′ consists of 7 nucleotide units. In some embodiments Y′ consists of 8 nucleotide units. In some embodiments Y′ consists of 9 nucleotide units. In certain embodiments, region Y′ consists of 10 nucleoside monomers. In certain embodiments, region Y′ consists or comprises 1-10 DNA monomers. In some embodiments Y′ comprises of from 1-9 DNA units, such as 2, 3, 4, 5, 6, 7, 8 or 9 DNA units. In some embodiments Y′ consists of DNA units. In some embodiments Y′ comprises of at least one LNA unit which is in the alpha-L configuration, such as 2, 3, 4, 5, 6, 7, 8 or 9 LNA units in the alpha-L-configuration. In some embodiments Y′ comprises of at least one alpha-L-oxy LNA unit or wherein all the LNA units in the alpha-L-configuration are alpha-L-oxy LNA units. In some embodiments the number of nucleotides present in X′-Y′-Z′ are selected from the group consisting of (nucleotide analogue units—region Y′—nucleotide analogue units): 1-8-1, 1-8-2, 2-8-1, 2-8-2, 3-8-3, 2-8-3, 3-8-2, 4-8-1, 4-8-2, 1-8-4, 2-8-4, or; 1-9-1, 1-9-2, 2-9-1, 2-9-2, 2-9-3, 3-9-2, 1-9-3, 3-9-1, 4-9-1, 1-9-4, or; 1-10- 1, 1-10-2, 2-10-1, 2-10-2, 1-10-3, 3-10-1. In some embodiments the number of nucleotides in X′-Y′-Z′ are selected from the group consisting of: 2-7-1, 1-7-2, 2-7-2, 3-7-3, 2-7-3, 3-7-2, 3-7-4, and 4-7-3. In certain embodiments, each of regions X′ and Y′ consists of three LNA monomers, and region Y′ consists of 8 or 9 or 10 nucleoside monomers, preferably DNA monomers. In some embodiments both X′ and Z′ consists of two LNA units each, and Y′ consists of 8 or 9 nucleotide units, preferably DNA units. In various embodiments, other gapmer designs include those where regions X′ and/or Z′ consists of 3, 4, 5 or 6 nucleoside analogues, such as monomers containing a 2′-O-methoxyethyl-ribose sugar (2′-MOE) or monomers containing a 2′-fluoro-deoxyribose sugar, and region Y′ consists of 8, 9, 10, 11 or 12 nucleosides, such as DNA monomers, where regions X′-Y′-Z′ have 3-9-3, 3-10-3, 5-10-5 or 4-12-4 monomers. Further gapmer designs are disclosed in WO 2007/146511A2, hereby incorporated by reference.

A LNA gapmer is a gapmer oligomer (region A) which comprises at least one LNA nucleotide. A preferred LNA gapmer oligomer is 12 to 16 nucleotides in length and comprises or consists of the oligomer motif of SEQ ID NO 2 with a 2-8-2 gapmer motif. SEQ ID NO 27 is an example of such an LNA gapmer oligomer.

### Internucleotide Linkages

The nucleoside monomers of the oligomers (e.g. first and second regions) described herein are coupled together via internucleoside linkage groups. Suitably, each monomer is linked to the 3′ adjacent monomer via a linkage group.

The person having ordinary skill in the art would understand that, in the context of the present invention, the 5′ monomer at the end of an oligomer does not comprise a 5′ linkage group, although it may or may not comprise a 5′ terminal group.

The terms “linkage group” or “internucleotide linkage” are intended to mean a group capable of covalently coupling together two nucleotides. Specific and preferred examples include phosphate groups and phosphorothioate groups.

The nucleotides of the oligomer of the invention or contiguous nucleotides sequence thereof are coupled together via linkage groups. Suitably each nucleotide is linked to the 3′ adjacent nucleotide via a linkage group.

Suitable internucleotide linkages include those listed within WO2007/031091, for example the internucleotide linkages listed on the first paragraph of page 34 of WO2007/031091 (hereby incorporated by reference).

It is, in some embodiments, other than the phosphodiester linkage(s) of region B (where present), the preferred to modify the internucleotide linkage from its normal phosphodiester to one that is more resistant to nuclease attack, such as phosphorothioate or boranophosphate—these two, being cleavable by RNase H, also allow that route of antisense inhibition in reducing the expression of the target gene.

Suitable sulphur (S) containing internucleotide linkages as provided herein may be preferred, such as phosphorothioate or phosphodithioate. Phosphorothioate internucleotide linkages are also preferred, particularly for the first region, such as in gapmers, mixmers, antimirs splice switching oligomers, and totalmers.

For gapmers, the internucleotide linkages in the oligomer may, for example be phosphorothioate or boranophosphate so as to allow RNase H cleavage of targeted RNA. Phosphorothioate is preferred, for improved nuclease resistance and other reasons, such as ease of manufacture.

In one aspect, with the exception of the phosphodiester linkage between the first and second region, and optionally within region B, the remaining internucleoside linkages of the oligomer of the invention, the nucleotides and/or nucleotide analogues are linked to each other by means of phosphorothioate groups. In some embodiments, at least 50%, such as at least 70%, such as at least 80%, such as at least 90% such as all the internucleoside linkages between nucleosides in the first region are other than phosphodiester (phosphate), such as are selected from the group consisting of phosphorothioate phosphorodithioate, or boranophosphate. In some embodiments, at least 50%, such as at least 70%, such as at least 80%, such as at least 90% such as all the internucleoside linkages between nucleosides in the first region are phosphorothioate.

WO09124238 refers to oligomeric compounds having at least one bicyclic nucleoside attached to the 3′ or 5′ termini by a neutral internucleoside linkage. The oligomers of the invention may therefore have at least one bicyclic nucleoside attached to the 3′ or 5′ termini by a neutral internucleoside linkage, such as one or more phosphotriester, methylphosphonate, MMI, amide-3, formacetal or thioformacetal. The remaining linkages may be phosphorothioate.

In a preferred embodiment all the internucleoside linkages linking the nucleotides of oligomers with the motif of SEQ ID NO 2 are phosphorothioate linkages.

### GalNAc Conjugate Moieties

Targeting to the liver can be greatly enhanced by the addition of a conjugate moiety (C). It is therefore desirable to use a conjugate moiety which enhances uptake and activity in hepatocytes. The enhancement of activity may be due to enhanced uptake or it may be due to enhanced potency of the compound in hepatocytes.

In some embodiments the carbohydrate moiety is not a linear carbohydrate polymer. The carbohydrate moiety may however be multi-valent, such as, for example 2, 3 or 4 identical or non-identical carbohydrate moieties may be covalently joined to the oligomer, optionally via a linker or linkers (such as region Y). In some embodiments the invention provides a conjugate comprising the oligomer of the invention and a carbohydrate conjugate moiety. In some embodiments the invention provides a conjugate comprising the oligomer of the invention and an asialoglycoprotein receptor targeting conjugate moiety, such as a GalNAc moiety, which may form part of a further region (referred to as region C).

The invention also provides modified oligonucleotides (such as LNA antisense) which are conjugated to an asialoglycoprotein receptor targeting moiety. In some embodiments, the conjugate moiety (such as the third region or region C) comprises an asialoglycoprotein receptor targeting moiety, such as galactose, galactosamine, N-formyl-galactosamine, N-acetylgalactosamine, N-propionyl-galactosamine, N-n-butanoyl-galactosamine, and N-isobutanoylgalactos-amine. In some embodiments the conjugate comprises 1 to 3 asialoglycoprotein receptor targeting moieties, such as N-acetylgalactosamine, preferably 2 to 3 asialoglycoprotein receptor targeting moieties N-acetylgalactosamine. More preferably the conjugate moiety comprises a galactose cluster, such as N-acetylgalactosamine trimer. In some embodiments, the conjugate moiety comprises a GalNAc (N-acetylgalactosamine), such as a mono-valent, di-valent, tri-valent or tetra-valent GalNAc. Trivalent GalNAc conjugates may be used to target the compound to the liver. GalNAc conjugates have been used with phosphodiester, methylphosphonate and PNA antisense oligonucleotides (e.g. U.S. Pat. No. 5,994,517 and Hangeland et al., Bioconjug Chem. 1995 November-December; 6(6):695-701, Biessen et al 1999 Biochem J. 340, 783-792 and Maier et al 2003 Bioconjug Chem 14, 18-29) and siRNAs (e.g. WO2009/126933, WO2012/089352 & WO2012/083046) and more recently with LNA and 2′-MOE modified nucleosides WO2014/076196 and WO 2014/179620. The GalNAc references and the specific conjugates used therein are hereby incorporated by reference, in particular the conjugate moieties in WO 2014/179620 are incorporated by reference. WO2012/083046 discloses siRNAs with GalNAc conjugate moieties which comprise cleavable pharmacokinetic modulators, which are suitable for use in the present invention, the preferred pharmacokinetic modulators are C16 hydrophobic groups such as palmitoyl, hexadec-8-enoyl, oleyl, (9E, 12E)-octadeca-9,12-dienoyl, dioctanoyl, and C16-C20 acyl. The '046 cleavable pharmacokinetic modulators may also be cholesterol.

The ‘targeting moieties (conjugate moieties) may be selected from the group consisting of: galactose, galactosamine, N-formyl-galactosamine, N-acetylgalactosamine, N-propionyl-galactosamine, N-n-butanoyl-galactosamine, N-iso-butanoylgalactos-amine, galactose cluster, and N-acetylgalactosamine trimer and may have a pharmacokinetic modulator selected from the group consisting of: hydrophobic group having 16 or more carbon atoms, hydrophobic group having 16-20 carbon atoms, palmitoyl, hexadec-8-enoyl, oleyl, (9E,12E)-octadeca-9, 12dienoyl, dioctanoyl, and C16-C20 acyl, and cholesterol. Certain GalNAc clusters disclosed in '046 include: (E)-hexadec-8-enoyl (C16), oleyl (C18), (9,E,12E)-octadeca-9,12-dienoyl (C18), octanoyl (C8), dodececanoyl (C12), C-20 acyl, C24 acyl, dioctanoyl (2×C8). The targeting moiety-pharmacokinetic modulator targeting moiety may be linked to the polynucleotide via a physiologically labile bond or, e.g. a disulfide bond, or a PEG linker. The invention also relates to the use of phosphodiester linkers between the oligomer and the conjugate group (these are referred to as region B herein, and suitably are positioned between the LNA oligomer and the carbohydrate conjugate group).

For targeting hepatocytes in liver, a preferred targeting ligand is a galactose cluster.

A galactose cluster comprises a molecule having e.g. comprising two to four terminal galactose derivatives. As used herein, the term galactose derivative includes both galactose and derivatives of galactose having affinity for the asialoglycoprotein receptor equal to or greater than that of galactose. A terminal galactose derivative is attached to a molecule through its C-I carbon. The asialoglycoprotein receptor (ASGPr) is unique to hepatocytes and binds branched galactose-terminal glycoproteins. A preferred galactose cluster has three terminal galactosamines or galactosamine derivatives each having affinity for the asialoglycoprotein receptor. A more preferred galactose cluster has three terminal N-acetyl-galactosamines. Other terms common in the art include tri-antennary galactose, tri-valent galactose and galactose trimer. It is known that tri-antennary galactose derivative clusters are bound to the ASGPr with greater affinity than bi-antennary or mono-antennary galactose derivative structures (Baenziger and Fiete, 1980, Cell, 22, 611-620; Connolly et al., 1982, 1. Biol. Chern., 257, 939-945). Multivalency is required to achieve nM affinity. According to WO 2012/083046 the attachment of a single galactose derivative having affinity for the asialoglycoprotein receptor does not enable functional delivery of the RNAi polynucleotide to hepatocytes in vivo when co-administered with the delivery polymer.

A galactose cluster may comprise two or preferably three galactose derivatives each linked to a central branch point. The galactose derivatives are attached to the central branch point through the C-I carbons of the saccharides. The galactose derivative is preferably linked to the branch point via linkers or spacers. A preferred spacer is a flexible hydrophilic spacer (U.S. Pat. No. 5,885,968; Biessen et al. J. Med. Chern. 1995 Vol. 39 p. 1538-1546). A preferred flexible hydrophilic spacer is a PEG spacer. A preferred PEG spacer is a PEG3 spacer. The branch point can be any small molecule which permits attachment of the three galactose derivatives and further permits attachment of the branch point to the oligomer. An exemplary branch point group is a di-lysine. A di-lysine molecule contains three amine groups through which three galactose derivatives may be attached and a carboxyl reactive group through which the di-lysine may be attached to the oligomer. Attachment of the branch point to oligomer may occur through a linker or spacer. A preferred spacer is a flexible hydrophilic spacer. A preferred flexible hydrophilic spacer is a PEG spacer. A preferred PEG spacer is a PEG3 spacer (three ethylene units). The galactose cluster may be attached to the 3′ or 5′ end of the oligomer using methods known in the art. In preferred embodiments the galactose cluster is linked to the 5′ end of the oligomer.

A preferred conjugate moiety is a galactose derivative, preferably an N-acetyl-galactosamine (GalNAc) conjugate moiety. More preferably a trivalent N-acetylgalactosamine moiety is used. Other saccharides having affinity for the asialoglycoprotein receptor may be selected from the list comprising: galactosamine, N-n-butanoylgalactosamine, and N-iso-butanoylgalactosamine. The affinities of numerous galactose derivatives for the asialoglycoprotein receptor have been studied (see for example: Jobst, S. T. and Drickamer, K. JB.C. 1996, 271, 6686) or are readily determined using methods typical in the art.

Conjugate moieties of the invention preferably comprises one to three N-acetylgalactosamine moiety(s). In some embodiments the conjugate moiety comprise a galactose cluster with three galactose moieties or derivatives thereof linked via a spacer to a branch point. Non-limiting examples of trivalent N-acetylgalactosamine clusters are shown in FIG. 13 and the figures below. A preferred conjugate moiety comprise three GalNAc moieties linked via a PEG spacer to a di-lysine. Preferably the PEG spacer is a 3PEG spacer.

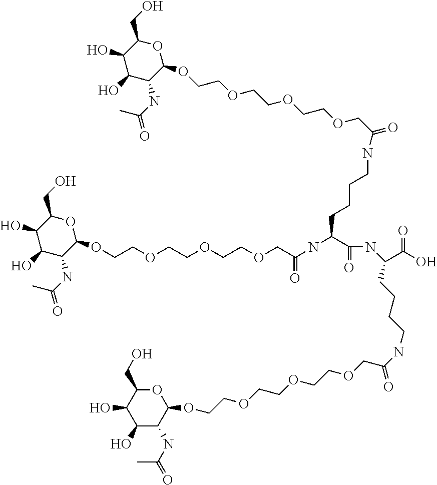 [SMILES: CC(=O)N[C@H]1[C@H](OCCOCCOCCOCC(=O)NCCCC[C@H](NC(=O)[C@H](CCCCNC(=O)COCCOCCOCCO[C@@H]2O[C@H](CO)[C@H](O)[C@H](O)[C@H]2NC(C)=O)NC(=O)COCCOCCOCCO[C@@H]2O[C@H](CO)[C@H](O)[C@H](O)[C@H]2NC(C)=O)C(=O)O)O[C@H](CO)[C@H](O)[C@@H]1O]

One embodiment of a Galactose cluster

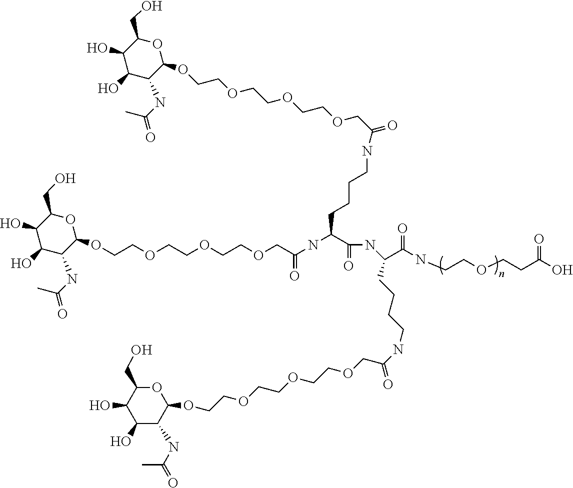

Galactose cluster with PEG spacer between branch point and nucleic acid

Further Examples of the conjugate of the invention are illustrated below:

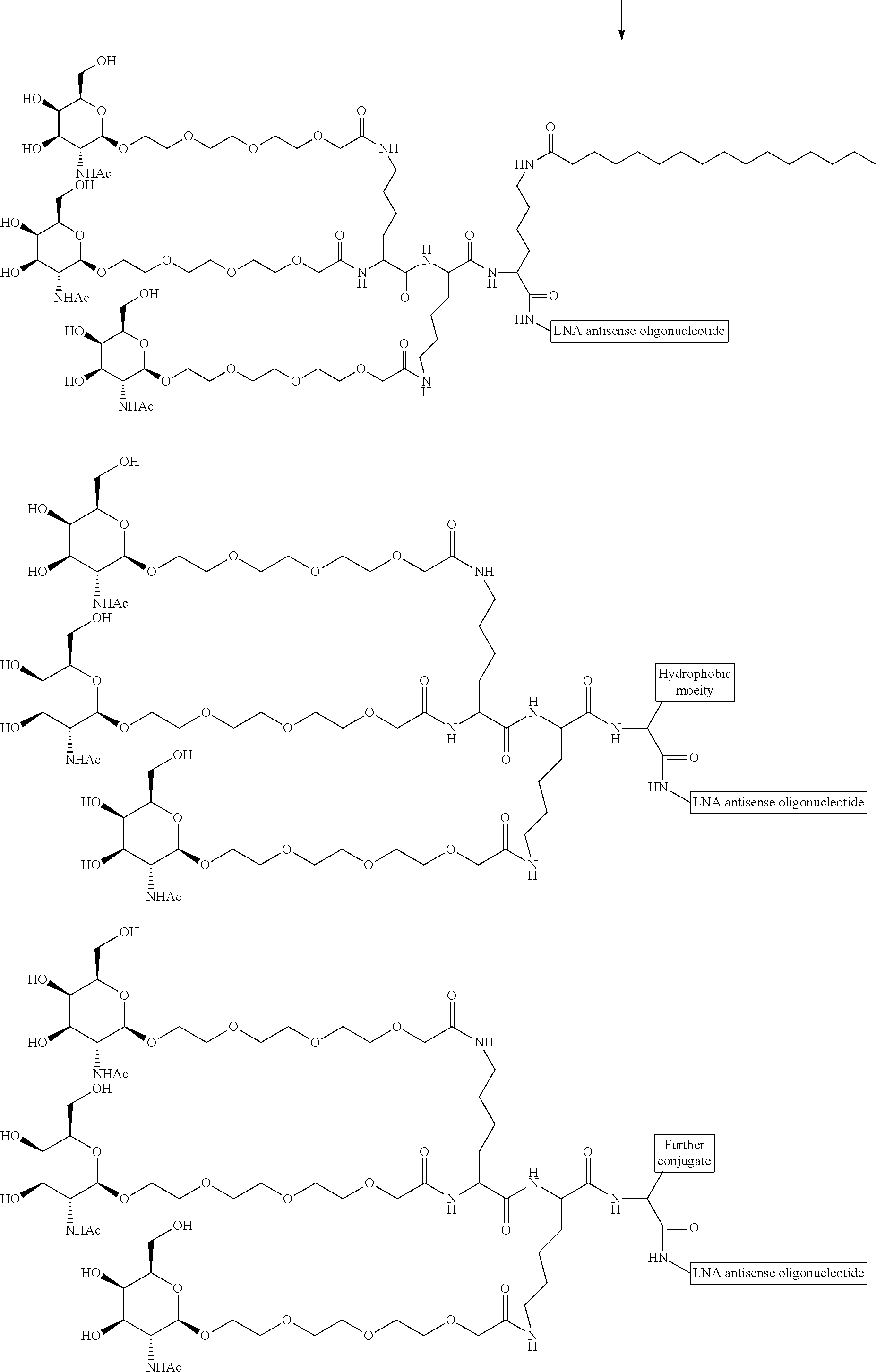

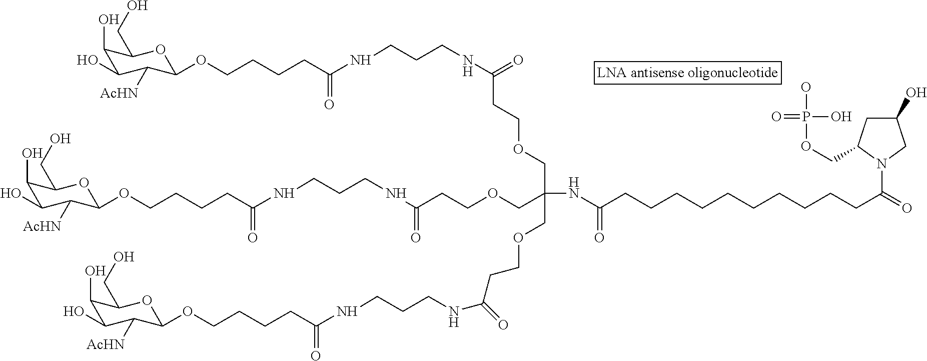 [SMILES: CC(=O)NC1C(OCCCCC(=O)NCCCNC(=O)CCOCC(COCCC(=O)NCCCNC(=O)CCCCOC2OC(CO)C(O)C(O)C2NC(C)=O)(COCCC(=O)NCCCNC(=O)CCCCOC2OC(CO)C(O)C(O)C2NC(C)=O)NC(=O)CCCCCCCCCCC(=O)N2C[C@H](O)C[C@H]2COP(=O)(O)O)OC(CO)C(O)C1O]

Where the hydrophobic or lipophilic (or further conjugate) moiety (i.e. pharmacokinetic modulator) in the above GalNAc cluster conjugates, when using LNA oligomers, such as LNA antisense oligonucleotides, is optional.

See FIG. 13 for specific GalNAc clusters used in the present study, Conj 1, 2, 3, 4 and Conj1a, 2a, 3a and 4a (which are shown with an optional C6 linker which joins the GalNAc cluster to the oligomer).

In a preferred embodiment of the invention the oligonucleotide conjugate corresponds to SEQ ID NO 29 or 31.

Each carbohydrate moiety of a GalNAc cluster (e.g. GalNAc) may therefore be joined to the oligomer via a spacer, such as (poly)ethylene glycol linker (PEG), such as a di, tri, tetra, penta, hexa-ethylene glycol linker. As is shown above the PEG moiety forms a spacer between the galctose sugar moiety and a peptide (trilysine is shown) linker.

In some embodiments, the GalNAc cluster comprises a peptide linker, e.g. a Tyr-Asp(Asp) tripeptide or Asp(Asp) dipeptide, which is attached to the oligomer (or to region Y or region B) via a biradical linker, for example the GalNAc cluster may comprise the following biradical linkers:

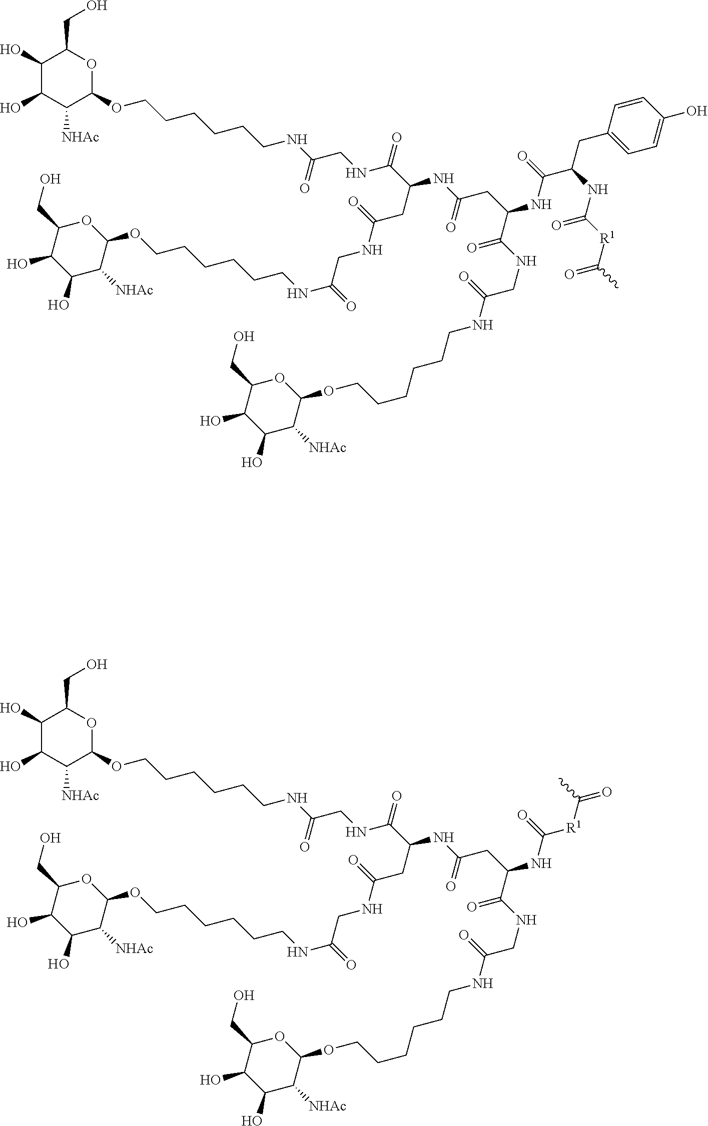

R¹ is a biradical preferably selected from —C₂H₄—, —C₃H₆—, —C₄H₈—, —C₅H₁₀—, —C₆H₁₂—, 1,4-cyclohexyl (—C₆H₁₀—), 1,4-phenyl (—C₆H₄—), —C₂H₄OC₂H₄—, —C₂H₄(OC₂H₄)₂— or —C₂H₄(OC₂H₄)₃—.

The carbohydrate conjugate (e.g. GalNAc), or carbohydrate-linker moiety (e.g. carbohydrate-PEG moiety) may be covalently joined (linked) to the oligomer via a branch point group such as, an amino acid, or peptide, which suitably comprises two or more amino groups (such as 3, 4, or 5), such as lysine, di-lysine or tri-lysine or tetra-lysine. A tri-lysine molecule contains four amine groups through which three carbohydrate conjugate groups, such as galactose & derivatives (e.g. GalNAc) and a further conjugate such as a hydrophobic or lipophilic moiety/group may be attached and a carboxyl reactive group through which the tri-lysine may be attached to the oligomer. The further conjugate, such as lipophilic/hydrophobic moiety may be attached to the lysine residue that is attached to the oligomer.

### Pharmacokinetic Modulators

The compound of the invention may further comprise one or more additional conjugate moieties, of which lipophilic or hydrophobic moieties are particularly interesting, such as when the conjugate group is a carbohydrate moiety. Such lipophilic or hydrophobic moieties may act as pharmacokinetic modulators, and may be covalently linked to either the carbohydrate conjugate, a linker linking the carbohydrate conjugate to the oligomer or a linker linking multiple carbohydrate conjugates (multi-valent) conjugates, or to the oligomer, optionally via a linker, such as a bio cleavable linker.

The oligomer or conjugate moiety may therefore comprise a pharmacokinetic modulator, such as a lipophilic or hydrophobic moiety. Such moieties are disclosed within the context of siRNA conjugates in WO2012/082046. The hydrophobic moiety may comprise a C8-C36 fatty acid, which may be saturated or un-saturated. In some embodiments, 010, C12, C14, C16, C18, C20, C22, C24, C26, C28, C30, C32 and C34 fatty acids may be used. The hydrophobic group may have 16 or more carbon atoms. Exemplary suitable hydrophobic groups may be selected from the group comprising: sterol, cholesterol, palmitoyl, hexadec-8-enoyl, oleyl, (9E, 12E)-octadeca-9,12-dienoyl, dioctanoyl, and C16-C20 acyl. According to WO′346, hydrophobic groups having fewer than 16 carbon atoms are less effective in enhancing polynucleotide targeting, but they may be used in multiple copies (e.g. 2×, such as 2×C8 or 010, C12 or C14) to enhance efficacy. Pharmacokinetic modulators useful as polynucleotide targeting moieties may be selected from the group consisting of: cholesterol, alkyl group, alkenyl group, alkynyl group, aryl group, aralkyl group, aralkenyl group, and aralkynyl group, each of which may be linear, branched, or cyclic. Pharmacokinetic modulators are preferably hydrocarbons, containing only carbon and hydrogen atoms. However, substitutions or heteroatoms which maintain hydrophobicity, for example fluorine, may be permitted.

In some embodiments, the conjugate is or may comprise a carbohydrate or comprises a carbohydrate group. In some embodiments, the carbohydrate is selected from the group consisting of galactose, lactose, n-acetylgalactosamine, mannose, and mannose-6-phosphate. In some embodiments, the conjugate group is or may comprise mannose or mannose-6-phosphate. Carbohydrate conjugates may be used to enhance delivery or activity in a range of tissues, such as liver and/or muscle. See, for example, EP1495769, WO99/65925, Yang et al., Bioconjug Chem (2009) 20(2): 213-21. Zatsepin & Oretskaya Chem Biodivers. (2004) 1(10): 1401-17.

Surprisingly, the present inventors have found that GalNAc conjugates for use with LNA oligomers do not require a pharmacokinetic modulator, and as such, in some embodiments, the GalNAc conjugate is not covalently linked to a lipophilic or hydrophobic moiety, such as those described here in, e.g. do not comprise a C8-C36 fatty acid or a sterol. The invention therefore also provides for LNA oligomer GalNAc conjugates which do not comprise a lipophilic or hydrophobic pharmacokinetic modulator or conjugate moiety/group.

In some embodiments, the conjugate moiety is hydrophilic. In some embodiments, the conjugate group does not comprise a lipophilic substituent group, such as a fatty acid substituent group, such as a C8-C26, such as a palmityl substituent group, or does not comprise a sterol, e.g. a cholesterol substituent group. In this regards, part of the invention is based on the surprising discovery that LNA oligomers GalNAc conjugates have remarkable pharmacokinetic properties even without the use of pharmacokinetic modulators, such as fatty acid substituent groups (e.g. >08 or >016 fatty acid groups).

### Lipophilic Conjugates

Lipophilic conjugates, such as sterols, stanols, and stains, such as cholesterol or as disclosed herein, may be used to enhance delivery of the oligonucleotide to, for example, the liver (typically hepatocytes).

In some embodiments, the conjugate group is or may comprise a sterol (for example, cholesterol, cholesteryl, cholestanol, stigmasterol, cholanic acid and ergosterol). In some embodiments the conjugate is or comprises tocopherol. In some embodiments, the conjugate is or may comprise cholesterol.

In some embodiments, the conjugate is, or may comprise a lipid, a phospholipid or a lipophilic alcohol, such as a cationic lipids, a neutral lipids, sphingolipids, and fatty acids such as stearic, oleic, elaidic, linoleic, linoleaidic, linolenic, and myristic acids. In some embodiments the fatty acid comprises a C4-C30 saturated or unsaturated alkyl chain. The alkyl chain may be linear or branched.

Lipophilic conjugate moieties can be used, for example, to counter the hydrophilic nature of an oligomeric compound and enhance cellular penetration.

Lipophilic moieties include, for example, sterols stanols, and steroids and related compounds such as cholesterol (U.S. Pat. No. 4,958,013 and Letsinger et al., Proc. Natl. Acad. Sci. USA, 1989, 86, 6553), thiocholesterol (Oberhauser et al, Nucl Acids Res., 1992, 20, 533), lanosterol, coprostanol, stigmasterol, ergosterol, calciferol, cholic acid, deoxycholic acid, estrone, estradiol, estratriol, progesterone, stilbestrol, testosterone, androsterone, deoxycorticosterone, cortisone, 17-hydroxycorticosterone, their derivatives, and the like. In some embodiments, the conjugate may be selected from the group consisting of cholesterol, thiocholesterol, lanosterol, coprostanol, stigmasterol, ergosterol, calciferol, cholic acid, deoxycholic acid, estrone, estradiol, estratriol, progesterone, stilbestrol, testosterone, androsterone, deoxycorticosterone, cortisone, and 17-hydroxycorticosterone. Other lipophilic conjugate moieties include aliphatic groups, such as, for example, straight chain, branched, and cyclic alkyls, alkenyls, and alkynyls. The aliphatic groups can have, for example, 5 to about 50, 6 to about 50, 8 to about 50, or 10 to about 50 carbon atoms. Example aliphatic groups include undecyl, dodecyl, hexadecyl, heptadecyl, octadecyl, nonadecyl, terpenes, bornyl, adamantyl, derivatives thereof and the like. In some embodiments, one or more carbon atoms in the aliphatic group can be replaced by a heteroatom such as O, S, or N (e.g., geranyloxyhexyl). Further suitable lipophilic conjugate moieties include aliphatic derivatives of glycerols such as alkylglycerols, bis(alkyl)glycerols, tris(alkyl)glycerols, monoglycerides, diglycerides, and triglycerides. In some embodiments, the lipophilic conjugate is di-hexyldecyl-rac-glycerol or 1,2-di-O-hexyldecyl-rac-glycerol (Manoharan et al., Tetrahedron Lett., 1995, 36, 3651; Shea, et al., Nuc. Acids Res., 1990, 18, 3777) or phosphonates thereof. Saturated and unsaturated fatty functionalities, such as, for example, fatty acids, fatty alcohols, fatty esters, and fatty amines, can also serve as lipophilic conjugate moieties. In some embodiments, the fatty functionalities can contain from about 6 carbons to about 30 or about 8 to about 22 carbons. Example fatty acids include, capric, caprylic, lauric, palmitic, myristic, stearic, oleic, linoleic, linolenic, arachidonic, eicosenoic acids and the like.

In further embodiments, lipophilic conjugate groups can be polycyclic aromatic groups having from 6 to about 50, 10 to about 50, or 14 to about 40 carbon atoms. Example polycyclic aromatic groups include pyrenes, purines, acridines, xanthenes, fluorenes, phenanthrenes, anthracenes, quinolines, isoquinolines, naphthalenes, derivatives thereof and the like. Other suitable lipophilic conjugate moieties include menthols, trityls (e.g., dimethoxytrityl (DMT)), phenoxazines, lipoic acid, phospholipids, ethers, thioethers (e.g., hexyl-S-tritylthiol), derivatives thereof and the like. Preparation of lipophilic conjugates of oligomeric compounds are well-described in the art, such as in, for example, Saison-Behmoaras et al, EMBO J., 1991, 10, 1111; Kabanov et al., FEBSLett., 1990, 259, 327; Svinarchuk et al, Biochimie, 1993, 75, 49; (Mishra et al., Biochim. Biophys. Acta, 1995, 1264, 229, and Manoharan et al., Tetrahedron Lett., 1995, 36, 3651.

Oligomeric compounds containing conjugate moieties with affinity for low density lipoprotein (LDL) can help provide an effective targeted delivery system. High expression levels of receptors for LDL on tumor cells makes LDL an attractive carrier for selective delivery of drugs to these cells (Rump, et al., Bioconjugate Chem., 1998, 9, 341; Firestone, Bioconjugate Chem., 1994, 5, 105; Mishra, et al., Biochim. Biophys. Acta, 1995, 1264, 229). Moieties having affinity for LDL include many lipophilic groups such as steroids (e.g., cholesterol), fatty acids, derivatives thereof and combinations thereof. In some embodiments, conjugate moieties having LDL affinity can be dioleyl esters of cholic acids such as chenodeoxycholic acid and lithocholic acid.

In some embodiments, the lipophillic conjugates may be or may comprise biotin. In some embodiments, the lipophilic conjugate may be or may comprise a glyceride or glyceride ester.

Lipophillic conjugates, such as sterols, stanols, and stains, such as cholesterol or as disclosed herein, may be used to enhance delivery of the oligonucleotide to, for example, the liver (typically hepatocytes).

The following references also refer to the use of lipophilic conjugates: Kobylanska et al., Acta Biochim Pol. (1999); 46(3): 679-91. Felber et al., Biomaterials (2012) 33(25): 599-65); Grijalvo et al., J Org Chem (2010) 75(20): 6806-13. Koufaki et al., Curr Med Chem (2009) 16(35): 4728-42. Godeau et al J. Med. Chem. (2008) 51(15): 4374-6 and WO 2013/033230.

In a preferred embodiment of the invention the oligonucleotide conjugate corresponds to SEQ ID NO 28.

Linkers (e.g. Region Y)

A linkage or linker is a connection between two atoms that links one chemical group or segment of interest to another chemical group or segment of interest via one or more covalent bonds. Conjugate moieties (or targeting or blocking moieties) can be attached to the oligomeric compound directly or through a linking moiety (linker or tether)—a linker. Linkers are bifunctional moieties that serve to covalently connect a third region, e.g. a conjugate moiety, to an oligomeric compound (such as to region B). In some embodiments, the linker comprises a chain structure or an oligomer of repeating units such as ethylene glycol or amino acid units. The linker can have at least two functionalities, one for attaching to the oligomeric compound and the other for attaching to the conjugate moiety. Example linker functionalities can be electrophilic for reacting with nucleophilic groups on the oligomer or conjugate moiety, or nucleophilic for reacting with electrophilic groups. In some embodiments, linker functionalities include amino, hydroxyl, carboxylic acid, thiol, phosphoramidate, phosphorothioate, phosphate, phosphite, unsaturations (e.g., double or triple bonds), and the like. Some example linkers include 8-amino-3,6-dioxaoctanoic acid (ADO), succinimidyl 4-(N-maleimidomethyl)cyclohexane-1-carboxylate (SMCC), 6-aminohexanoic acid (AHEX or AHA), 6-aminohexyloxy, 4-aminobutyric acid, 4-aminocyclohexylcarboxylic acid, succinimidyl 4-(N-maleimidomethyl)cyclohexane-1-carboxy-(6-amido-caproate) (LCSMCC), succinimidyl m-maleimido-benzoylate (MBS), succinimidyl N-e-maleimido-caproylate (EMCS), succinimidyl 6-(beta-maleimido-propionamido) hexanoate (SMPH), succinimidyl N-(a-maleimido acetate) (AMAS), succinimidyl 4-(p-maleimidophenyl)butyrate (SMPB), beta-alanine (beta-ALA), phenylglycine (PHG), 4-aminocyclohexanoic acid (ACHC), beta-(cyclopropyl) alanine (beta-CYPR), amino dodecanoic acid (ADC), alylene diols, polyethylene glycols, amino acids, and the like.

A wide variety of further linker groups are known in the art that can be useful in the attachment of conjugate moieties to oligomeric compounds. A review of many of the useful linker groups can be found in, for example, Antisense Research and Applications, S. T. Crooke and B. Lebleu, Eds., CRC Press, Boca Raton, Fla., 1993, p. 303-350. A disulfide linkage has been used to link the 3′ terminus of an oligonucleotide to a peptide (Corey, et al., Science 1987, 238, 1401; Zuckermann, et al, J Am. Chem. Soc. 1988, 110, 1614; and Corey, et al., J Am. Chem. Soc. 1989, 111, 8524). Nelson, et al., Nuc. Acids Res. 1989, 17, 7187 describe a linking reagent for attaching biotin to the 3′-terminus of an oligonucleotide. This reagent, N-Fmoc-O-DMT-3-amino-1,2-propanediol is commercially available from Clontech Laboratories (Palo Alto, Calif.) under the name 3′-Amine. It is also commercially available under the name 3′-Amino-Modifier reagent from Glen Research Corporation (Sterling, Va.). This reagent was also utilized to link a peptide to an oligonucleotide as reported by Judy, et al., Tetrahedron Letters 1991, 32, 879. A similar commercial reagent for linking to the 5′-terminus of an oligonucleotide is 5′-Amino-Modifier C6. These reagents are available from Glen Research Corporation (Sterling, Va.). These compounds or similar ones were utilized by Krieg, et al, Antisense Research and Development 1991, 1, 161 to link fluorescein to the 5′-terminus of an oligonucleotide. Other compounds such as acridine have been attached to the 3′-terminal phosphate group of an oligonucleotide via a polymethylene linkage (Asseline, et al., Proc. Natl. Acad. Sci. USA 1984, 81, 3297). [0074] Any of the above groups can be used as a single linker or in combination with one or more further linkers.

Linkers and their use in preparation of conjugates of oligomeric compounds are provided throughout the art such as in WO 96/11205 and WO 98/52614 and U.S. Pat. Nos. 4,948,882; 5,525,465; 5,541,313; 5,545,730; 5,552,538; 5,580,731; 5,486,603; 5,608,046; 4,587,044; 4,667,025; 5,254,469; 5,245,022; 5,112,963; 5,391,723; 5,510475; 5,512,667; 5,574,142; 5,684,142; 5,770,716; 6,096,875; 6,335,432; and 6,335,437, Wo2012/083046 each of which is incorporated by reference in its entirety.

As used herein, a physiologically labile bond is a labile bond that is cleavable under conditions normally encountered or analogous to those encountered within a mammalian body (also referred to as a cleavable linker). Physiologically labile linkage groups are selected such that they undergo a chemical transformation (e.g., cleavage) when present in certain physiological conditions. Mammalian intracellular conditions include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic or hydrolytic enzymes. In some embodiments, the cleavable linker is susceptible to nuclease(s) which may for example, be expressed in the target cell—and as such, as detailed herein, the linker may be a short region (e.g. 1-10) phosphodiester linked nucleosides, such as DNA nucleosides,

Chemical transformation (cleavage of the labile bond) may be initiated by the addition of a pharmaceutically acceptable agent to the cell or may occur spontaneously when a molecule containing the labile bond reaches an appropriate intra- and/or extra-cellular environment. For example, a pH labile bond may be cleaved when the molecule enters an acidified endosome. Thus, a pH labile bond may be considered to be an endosomal cleavable bond. Enzyme cleavable bonds may be cleaved when exposed to enzymes such as those present in an endosome or lysosome or in the cytoplasm. A disulfide bond may be cleaved when the molecule enters the more reducing environment of the cell cytoplasm. Thus, a disulfide may be considered to be a cytoplasmic cleavable bond. As used herein, a pH-labile bond is a labile bond that is selectively broken under acidic conditions (pH<7). Such bonds may also be termed endosomally labile bonds, since cell endosomes and lysosomes have a pH less than 7.

### Oligomer Linked Biocleavable Conjugates

The oligomeric compound may optionally, comprise a second region (region B) which is positioned between the oligomer (referred to as region A) and the conjugate (referred to as region C). Region B may be a linker such as a cleavable linker (also referred to as a physiologically labile linkage). (see Example 6)

### Nuclease Susceptible Physiological Labile Linkages:

In some embodiments, the oligomer (also referred to as oligomeric compound) of the invention (or conjugate) comprises three regions:
i) a first region (region A), which comprises an oligonucleotide with motif of SEQ ID NO 2;
ii) a second region (region B) which comprises a biocleavable linker
iii) a third region (C) which comprises a conjugate moiety, a targeting moiety, an activation moiety, wherein the third region is covalent linked to the second region.

The oligonucleotide conjugate of the invention can be constructed such that a lysine linker (region B) joins the N-acetylgalactosamine group(s) (region C) and the oligomer (region A) optionally via a further linker Y. The further linker Y is inserted between the lysine linker and the oligomer. The N-acetylgalactosamine group(s) joined to a lysine linker can also be considered as a conjugate moiety (region C) where Region B is embedded in Region C. The linker Y can therefore be between region C and A.

For trivalent GalNAc conjugates, each GalNAc moiety may be joined to the biocleavable linker (e.g. a di-lysine or tri-lysine linker) which is further covalently joined to the oligomer (SEQ ID NO 2 or SEQ ID NO: 27). Optionally a further linker (Y) can be inserted between the biocleavable lysine linker and the oligomer. Linker Y can for example be a fatty acid such as a C6 linker. In addition to linker Y a physiologically cleavable linker Region B can be inserted between the oligomer and linker Y.

In some embodiments, region B may be a phosphate nucleotide linker. For example such linkers may be used when the conjugate is a sterol, such as cholesterol or tocopherol. Phosphate nucleotide linkers may also be used for other conjugates, for example carbohydrate conjugates, such as GalNAc.

In a preferred embodiment the oligonucleotide conjugate comprises three N-acetylgalactosamine units linked to a spacer and a C6 linker connecting the oligomer to the di-lysine linker. Examples of such constructs are shown in FIGS. 13 and 13A. In a further embodiment a PEG spacer is inserted between the GalNAc moiety and the lysine linker (e.g. Conl1a and Conj2a). In a further embodiment a physiologically labile nucleotide linker can be inserted between the C6 linker and the oligomer.

### Peptide Linkers

In some embodiments, the biocleavable linker (region B) is a peptide, such as a trilysine peptide linker which may be used in a polyGalNAc conjugate, such as a trimeric GalNAc conjugate.

Other linkers known in the art which may be used, include disulfide linkers.

### Phosphate Nucleotide Linkers

In some embodiments, region B comprises between 1-6 nucleotides, which is covalently linked to the 5′ or 3′ nucleotide of the first region, such as via a internucleoside linkage group such as a phosphodiester linkage, wherein either
a. the internucleoside linkage between the first and second region is a phosphodiester linkage and the nucleoside of the second region [such as immediately] adjacent to the first region is either DNA or RNA; and/or
b. at least 1 nucleoside of the second region is a phosphodiester linked DNA or RNA nucleoside;

In some embodiments, region A and region B form a single contiguous nucleotide sequence of 13-16 nucleotides in length.

In some aspects the internucleoside linkage between the first and second regions may be considered part of the second region.

In some embodiments, there is a phosphorus containing linkage group between the second and third region. The phosphorus linkage group, may, for example, be a phosphate (phosphodiester), a phosphorothioate, a phosphorodithioate or a boranophosphate group. In some embodiments, this phosphorus containing linkage group is positioned between the second region and a linker region which is attached to the third region. In some embodiments, the phosphate group is a phosphodiester.

Therefore, in some aspects the oligomeric compound comprises at least two phosphodiester groups, wherein at least one is as according to the above statement of invention, and the other is positioned between the second and third (conjugate) regions, optionally between a linker group and the second region.

The antisense oligonucleotide may be or may comprise the first region, and optionally the second region. In this respect, in some embodiments, region B may form part of a contiguous nucleobase sequence which is complementary to the (nucleic acid) target. In other embodiments, region B may lack complementarity to the target.

In some embodiments, at least two consecutive nucleosides of the second region are DNA nucleosides (such as at least 3 or 4 or 5 consecutive DNA nucleotides).

In such an embodiment, the oligonucleotide of the invention may be described according to the following formula:

5′-A-PO-B[Y)X-3′ or 3′-A-PO-B[Y)X-5′

wherein A is region A, PO is a phosphodiester linkage, B is region B, Y is an optional linkage group, and X is a conjugate, a targeting, a blocking group or a reactive or activation group.

In some embodiments, region B comprises 3′-5′ or 5′-3′: i) a phosphodiester linkage to the 5′ nucleoside of region A, ii) a DNA or RNA nucleoside, such as a DNA nucleoside, and iii) a further phosphodiester linkage

5′-A-PO-B-PO-3′ or 3′-A-PO-B-PO-5′

The further phosphodiester linkage link the region B nucleoside with one or more further nucleoside, such as one or more DNA or RNA nucleosides, or may link to X (is a conjugate, a targeting or a blocking group or a reactive or activation group) optionally via a linkage group (Y).

In some embodiments, region B comprises 3′-5′ or 5′-3′: i) a phosphodiester linkage to the 5′ nucleoside of region A, ii) between 2-10 DNA or RNA phosphodiester linked nucleosides, such as a DNA nucleoside, and optionally iii) a further phosphodiester linkage:

5′-A-[PO-B]n [Y]-X 3′ or 3′-A-[PO-B]n-[Y]-X 5′

5′-A-[PO-B]n-PO-[Y]-X 3′ or 3′-A-[PO-B]n-PO-[Y]-X 5′

Wherein A represent region A, [PO-B]n represents region B, wherein n is 1-10, such as 1, 2, 3, 4, 5, 6, 7, 8, 9 or 10, PO is an optional phosphodiester linkage group between region B and X (or Y if present).

In some embodiments the invention provides compounds according to (or comprising) one of the following formula:

5′[Region A]-PO-[region B]3′-Y-X

5′[Region A]-PO-[region B]-PO 3′-Y-X

5′[Region A]-PO-[region B]3′-X

5′[Region A]-PO-[region B]-PO 3′-X

3′[Region A]-PO-[region B]5′-Y-X

3′[Region A]-PO-[region B]-PO 5′-Y-X

3′[Region A]-PO-[region B]5′-X

3′[Region A]-PO-[region B]-PO 5′-X

Region B, may for example comprise or consist of:

5′ DNA3′

3′ DNA 5′

5′ DNA-PO-DNA-3′

3′ DNA-PO-DNA-5′

5′ DNA-PO-DNA-PO-DNA 3′

3′ DNA-PO-DNA-PO-DNA 5′

5′ DNA-PO-DNA-PO-DNA-PO-DNA 3′

3′ DNA-PO-DNA-PO-DNA-PO-DNA 5′

5′ DNA-PO-DNA-PO-DNA-PO-DNA-PO-DNA 3′

3′ DNA-PO-DNA-PO-DNA-PO-DNA-PO-DNA 5′

It should be recognized that phosphate linked biocleavable linkers may employ nucleosides other than DNA and RNA. Bio cleavable nucleotide linkers may, for example, be identified using the assays in Example 7.

In some embodiments, the compound of the invention comprises a biocleavable linker (also referred to as the physiologically labile linker, Nuclease Susceptible Physiological Labile Linkages, or nuclease susceptible linker), for example the phosphate nucleotide linker (such as region B) or a peptide linker, which joins the oligomer (or contiguous nucleotide sequence or region A), to a conjugate moiety (or region C).

The susceptibility to cleavage in the assays shown in Example 7 can be used to determine whether a linker is biocleavable or physiologically labile.

Biocleavable linkers according to the present invention refers to linkers which are susceptible to cleavage in a target tissue (i.e. physiologically labile), for example liver and/or kidney. It is preferred that the cleavage rate seen in the target tissue is greater than that found in blood serum. Suitable methods for determining the level (%) of cleavage in tissue (e.g. liver or kidney) and in serum are found in example 6. In some embodiments, the biocleavable linker (also referred to as the physiologically labile linker, or nuclease susceptible linker), such as region B, in a compound of the invention, are at least about 20% cleaved, such as at least about 30% cleaved, such as at least about 40% cleaved, such as at least about 50% cleaved, such as at least about 60% cleaved, such as at least about 70% cleaved, such as at least about 75% cleaved, in the liver or kidney homogenate assay of Example 7. In some embodiments, the cleavage (%) in serum, as used in the assay in Example 7, is less than about 30%, is less than about 20%, such as less than about 10%, such as less than 5%, such as less than about 1%.

In some embodiments, which may be the same of different, the biocleavable linker (also referred to as the physiologically labile linker, or nuclease susceptible linker), such as region B, in a compound of the invention, are susceptible to 51 nuclease cleavage. Susceptibility to 51 cleavage may be evaluated using the 51 nuclease assay shown in Example 7. In some embodiments, the biocleavable linker (also referred to as the physiologically labile linker, or nuclease susceptible linker), such as region B, in a compound of the invention, are at least about 30% cleaved, such as at least about 40% cleaved, such as at least about 50% cleaved, such as at least about 60% cleaved, such as at least about 70% cleaved, such as at least about 80% cleaved, such as at least about 90% cleaved, such as at least 95% cleaved after 120 min incubation with S1 nuclease according to the assay used in Example 6.

### Sequence Selection in the Second Region:

In some embodiments, region B does not form a complementary sequence when the oligonucleotide region A and B is aligned to the complementary target sequence.

In some embodiments, region B does form a complementary sequence when the oligonucleotide region A and B is aligned to the complementary target sequence. In this respect region A and B together may form a single contiguous sequence which is complementary to the target sequence.

In some embodiments, the sequence of bases in region B is selected to provide an optimal endonuclease cleavage site, based upon the predominant endonuclease cleavage enzymes present in the target tissue or cell or sub-cellular compartment. In this respect, by isolating cell extracts from target tissues and non-target tissues, endonuclease cleavage sequences for use in region B may be selected based upon a preferential cleavage activity in the desired target cell (e.g. liver/hepatocytes) as compared to a non-target cell (e.g. kidney). In this respect, the potency of the compound for target down-regulation may be optimized for the desired tissue/cell.

In some embodiments region B comprises a dinucleotide of sequence AA, AT, AC, AG, TA, TT, TC, TG, CA, CT, CC, CG, GA, GT, GC, or GG, wherein C may be 5-mthylcytosine, and/or T may be replaced with U. In some embodiments region B comprises a trinucleotide of sequence AAA, AAT, AAC, AAG, ATA, ATT, ATC, ATG, ACA, ACT, ACC, ACG, AGA, AGT, AGC, AGG, TAA, TAT, TAC, TAG, TTA, TTT, TTC, TAG, TCA, TCT, TCC, TCG, TGA, TGT, TGC, TGG, CAA, CAT, CAC, CAG, CTA, CTG, CTC, CTT, CCA, CCT, CCC, CCG, CGA, CGT, CGC, CGG, GAA, GAT, GAC, CAG, GTA, GTT, GTC, GTG, GCA, GCT, GCC, GCG, GGA, GGT, GGC, and GGG wherein C may be 5-mthylcytosine and/or T may be replaced with U. In some embodiments region B comprises a trinucleotide of sequence AAAX, AATX, AACX, AAGX, ATAX, ATTX, ATCX, ATGX, ACAX, ACTX, ACCX, ACGX, AGAX, AGTX, AGCX, AGGX, TAAX, TATX, TACX, TAGX, TTAX, TTTX, TTCX, TAGX, TCAX, TCTX, TCCX, TCGX, TGAX, TGTX, TGCX, TGGX, CAAX, CATX, CACX, CAGX, CTAX, CTGX, CTCX, CTTX, CCAX, CCTX, CCCX, CCGX, CGAX, CGTX, CGCX, CGGX, GAAX, GATX, GACX, CAGX, GTAX, GTTX, GTCX, GTGX, GCAX, GCTX, GCCX, GCGX, GGAX, GGTX, GGCX, and GGGX, wherein X may be selected from the group consisting of A, T, U, G, C and analogues thereof, wherein C may be 5-mthylcytosine and/or T may be replaced with U. It will be recognized that when referring to (naturally occurring) nucleobases A, T, U, G, C, these may be substituted with nucleobase analogues which function as the equivalent natural nucleobase (e.g. base pair with the complementary nucleoside).

### Amino Alkyl Intermediates

The invention further provides for the LNA oligomer intermediates which comprise an antisense LNA oligomer (SEQ ID NO 2) which comprises an (e.g. terminal, 5′ or 3′) amino alkyl, such as a C2-C36 amino alkyl group, including, for example C6 and C12 amino alkyl groups. The amino alkyl group may be added to the LNA oligomer as part of standard oligonucleotide synthesis, for example using a (e.g. protected) amino alkyl phosphoramidite. The linkage group between the amino alkyl and the LNA oligomer may for example be a phosphorothioate or a phosphodiester, or one of the other nucleoside linkage groups referred to herein, for example. The amino alkyl group may be covalently linked to, for example, the 5′ or 3′ of the LNA oligomer, such as by the nucleoside linkage group, such as phosphorothioate or phosphodiester linkage.

The invention also provides a method of synthesis of the LNA oligomer comprising the sequential synthesis of the LNA oligomer, such as solid phase oligonucleotide synthesis, comprising the step of adding an amino alkyl group to the oligomer, such as e.g. during the first or last round of oligonucleotide synthesis. The method of synthesis may further comprise the step of reacting a conjugate to the amino alkyl-LNA oligomer (the conjugation step). The a conjugate may comprise suitable linkers and/or branch point groups, and optionally further conjugate groups, such as hydrophobic or lipophilic groups, as described herein. The conjugation step may be performed whilst the oligomer is bound to the solid support (e.g. after oligonucleotide synthesis, but prior to elution of the oligomer from the solid support), or subsequently (i.e. after elution). The invention provides for the use of an amino alkyl linker in the synthesis of the oligomer of the invention.

### Method of Manufacture/Synthesis

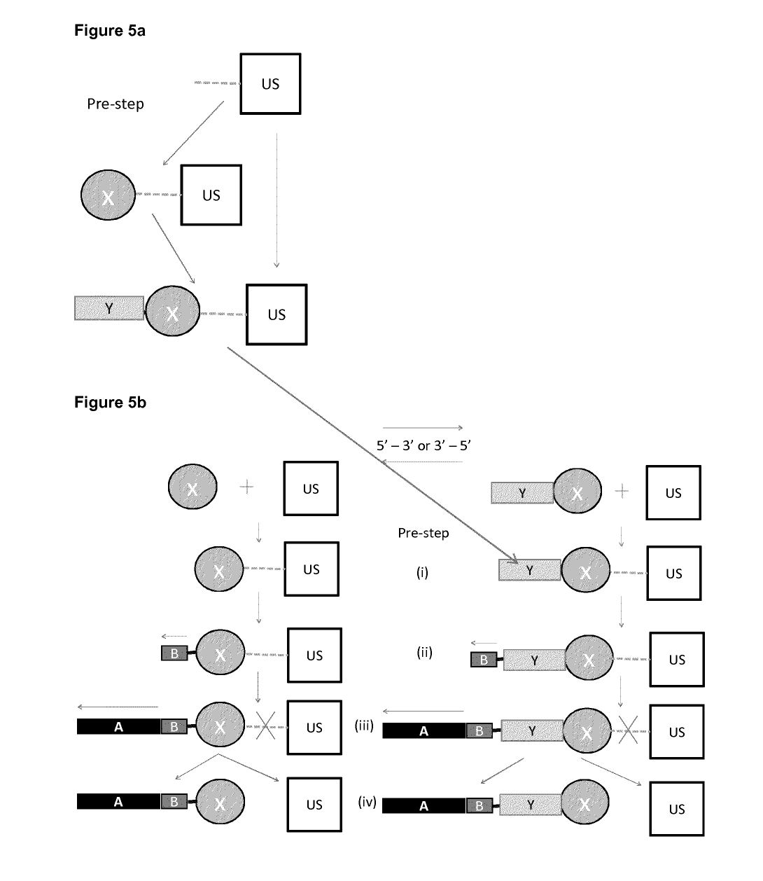

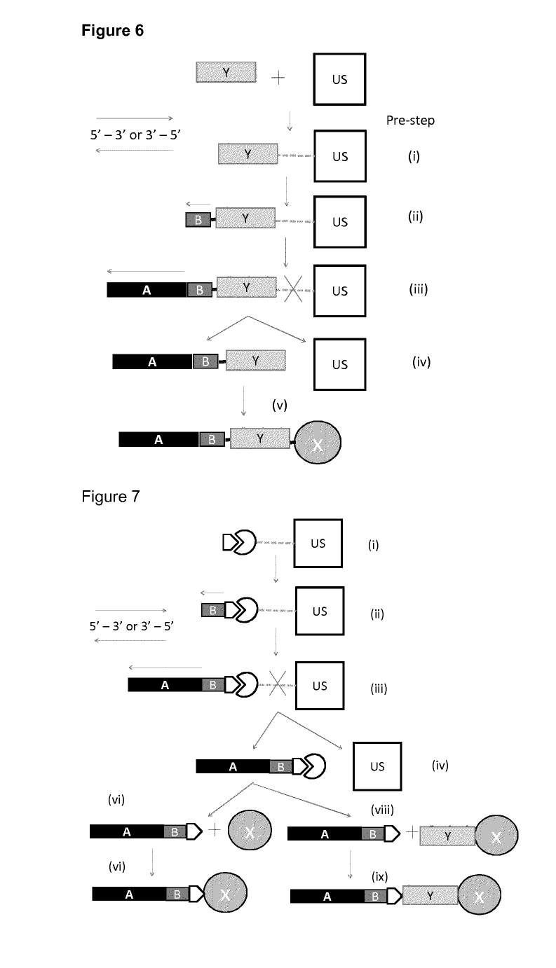

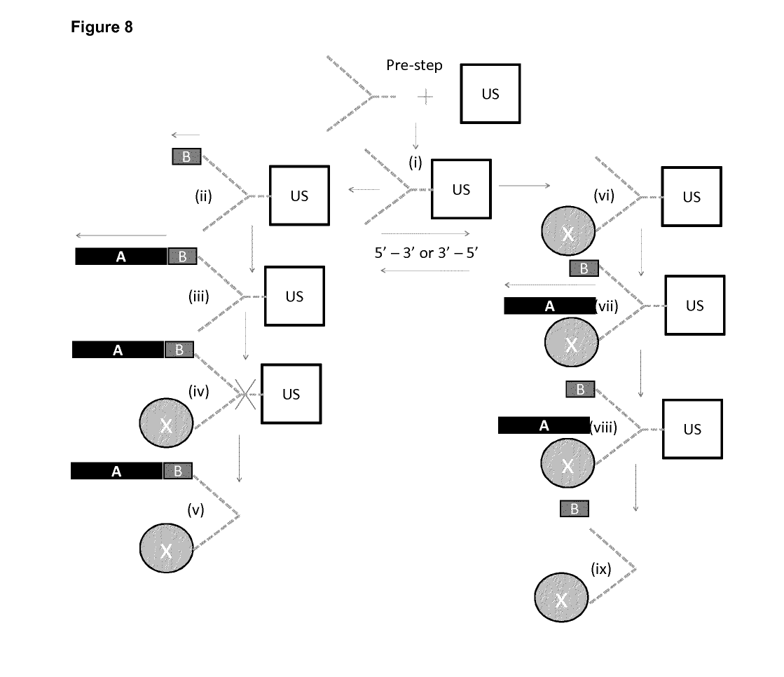

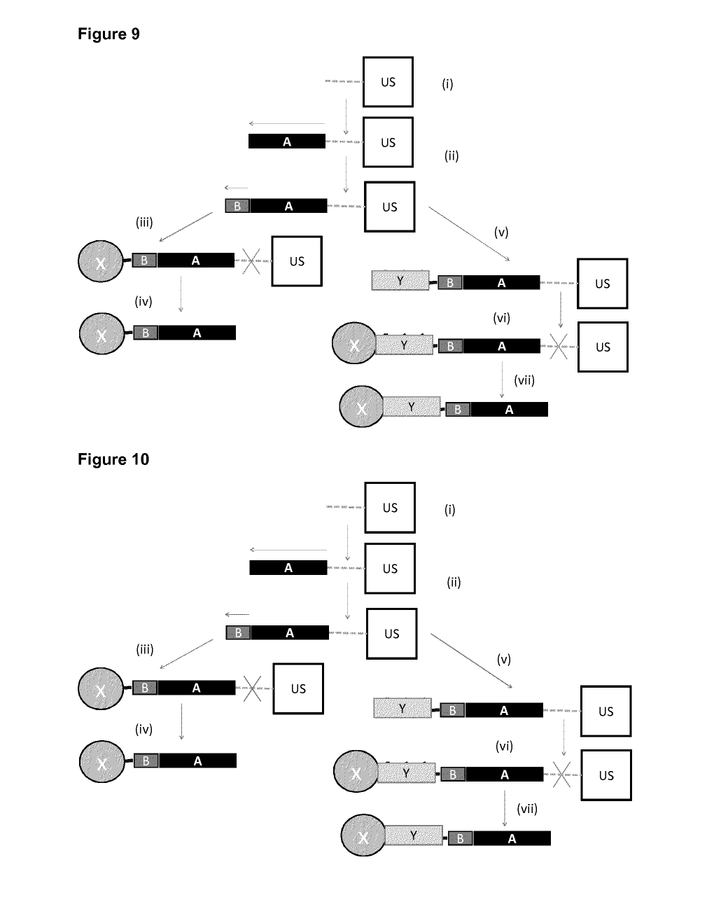

The invention provides for a method of synthesizing (or manufacture) of an oligonucleotide conjugate of the invention, said method comprising either:
a) a step of providing a solid phase oligonucleotide synthesis support to which one of the following is attached third region:
   i) a linker group (-Y-)
   ii) a group (X-) selected from the group consisting of a conjugate, a targeting group, a blocking group, a reactive group (e.g. an amine or an alcohol) or an activation group
   iii) an -Y -X group
and
b) a step of sequential oligonucleotide synthesis of region B followed by region A, and/or:
c) a step of sequential oligonucleotide synthesis of a first region (A) and a second region (B), wherein the synthesis step is followed by
d) a step of adding a third region [phosphoramidite comprising]
   i) a linker group (-Y-)
   ii) a group (X-) selected from the group consisting of a conjugate, a targeting group, a blocking group, a reactive group [e.g. an amine or an alcohol] or an activation group
iii) an -Y -X group
followed by
e) the cleavage of the oligomeric compound from the solid phase support wherein, optionally said method further comprises a further step selected from:
f) wherein the third group is an activation group, the step of activating the activation group to produce a reactive group, followed by adding a conjugate, a blocking, or targeting group to the reactive group, optionally via a linker group (Y);
g) wherein the third region is a reactive group, the step of adding a conjugate, a blocking, or targeting group to the reactive group, optionally via a linker group (Y).
h) wherein the third region is a linker group (Y), the step of adding a conjugate, a blocking, or targeting group to the linker group (Y)

wherein steps f), g) or h) are performed either prior to or subsequent to cleavage of the oligomeric compound from the oligonucleotide synthesis support. In some embodiments, the method may be performed using standard phosphoramidite chemistry, and as such the region X and/or region X or region X and Y may be provided, prior to incorporation into the oligomer, as a phosphoramidite. Please see FIGS. 5-10 which illustrate non-limiting aspects of the method of producing the oligonucleotide conjugate of the invention.

The invention provides for a method of synthesizing (or manufacture) of an oligonucleotide conjugate of the invention, said method comprising a step of sequential oligonucleotide synthesis of an oligomer with the oligomer with the oligonucleotide motif of SEQ ID NO 2 (region (A)) and optionally a second region (B), wherein the synthesis step is followed by a step of adding a conjugate moiety phosphoramidite comprising a N-acetylgalactosamine moiety or a sterol moiety followed by the cleavage of the oligomeric compound from the solid phase support. The N-acetylgalactosamine moiety or a sterol moiety can be selected from those described in the corresponding sections. In a preferred embodiment the N-acetylgalactosamine moiety is selected from Conj1a or Conj2a.

It is however recognized that the conjugate moiety phosphoramidite comprising a N-acetylgalactosamine moiety or a sterol moiety may be added after the cleavage from the solid support. Alternatively, the method of synthesis may comprise the steps of synthesizing the oligomer with the oligonucleotide motif of SEQ ID NO 2 (region (A)) and optionally a second region (B), followed by the cleavage of the oligomer from the support, with a subsequent step of adding a conjugate moiety comprising a N-acetylgalactosamine moiety or a sterol moiety to the oligomer. The addition of the third region may be achieved, by example, by adding an amino phosphoramidite unit in the final step of oligomer synthesis (on the support), which can, after cleavage from the support, be used to join to a conjugate moiety comprising a N-acetylgalactosamine moiety or a sterol moiety to the oligomer. In the embodiments where the cleavable linker is not a nucleotide region, region B may be a non-nucleotide cleavable linker for example a peptide linker, which may form part of the conjugate moiety (also referred to as region C) or be region Y (or part thereof).

In some embodiments of the method, the conjugate moiety (e.g. the GalNAc conjugate) comprises an activation group, (an activated functional group) and in the method of synthesis the activated conjugate is added to the oligomer, such as an amino linked oligomer. The amino group may be added to the oligomer by standard phosphoramidite chemistry, for example as the final step of oligomer synthesis (which typically will result in amino group at the 5′ end of the oligomer). For example during the last step of the oligonucleotide synthesis a protected amino-alkyl phosphoramidite is used, for example a TFA-aminoC6 phosphoramidite (6-(Trifluoroacetylamino)-hexyl-(2-cyanoethyl)-(N,N-diisopropyl)-phosphoramidite).

The conjugate moiety (e.g. a GalNac conjugate) may be activated via NHS ester method and then the aminolinked oligomer is added. For example a N-hydroxysuccinimide (NHS) may be used as activating group for the conjugate moiety, such as a GalNAc.

The invention provides an oligonucleotide conjugate prepared by the method of the invention.

In some embodiments, the conjugate moiety comprising a sterol moiety may be covalently joined (linked) to region B via a phosphate nucleoside linkage, such as those described herein, including phosphodiester or phosphorothioate, or via an alternative group, such as a triazol group.

In some embodiments, the internucleoside linkage between the first and second region is a phosphodiester linked to the first (or only) DNA or RNA nucleoside of the second region, or region B comprises at least one phosphodiester linked DNA or RNA nucleoside.

The second region may, in some embodiments, comprise further DNA or RNA nucleosides which may be phosphodester linked. The second region is further covalently linked to a third region which may, for example, be a conjugate, a targeting group a reactive group, and/or a blocking group.

In some aspects, the present invention is based upon the provision of a labile region, the second region, linking the first region, e.g. an antisense oligonucleotide, and a conjugate moiety. The labile region comprises at least one phosphodiester linked nucleoside, such as a DNA or RNA nucleoside, such as 1, 2, 3, 4, 5, 6, 7, 8, 9 or 10 phosphodiester linked nucleosides, such as DNA or RNA. In some embodiments, the oligomeric compound comprises a cleavable (labile) linker. In this respect the cleavable linker is preferably present in region B (or in some embodiments, between region A and B).

Alternatively stated, in some embodiments, the invention provides a non-phosphodiester linked, such as a phosphorothioate linked, oligonucleotide (e.g. an antisense oligonucleotide) which has at least one terminal (5′ and/or 3′) DNA or RNA nucleoside linked to the adjacent nucleoside of the oligonucleotide via a phosphodiester linkage, wherein the terminal DNA or RNA nucleoside is further covalently linked to a conjugate moiety, a targeting moiety or a blocking moiety, optionally via a linker moiety.

### Compositions

The oligomer, in particular the oligonucleotide conjugates of the invention may be used in pharmaceutical formulations and compositions. Suitably, such compositions comprise a pharmaceutically acceptable diluent, carrier, salt or adjuvant. WO2007/031091 provides suitable and preferred pharmaceutically acceptable diluent, carrier and adjuvants—which are hereby incorporated by reference. Suitable dosages, formulations, administration routes, compositions, dosage forms, combinations with other therapeutic agents, pro-drug formulations are also provided in WO2007/031091—which are also hereby incorporated by reference.

Antisense oligonucleotide conjugates of the invention may be mixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

An Antisense oligonucleotide conjugate can be utilized in pharmaceutical compositions by combining the antisense oligonucleotide conjugate compound with a suitable pharmaceutically acceptable diluent or carrier. A pharmaceutically acceptable diluent includes phosphate-buffered saline (PBS). PBS is a diluent suitable for use in compositions to be delivered parenterally.

Pharmaceutical compositions comprising antisense oligonucleotide conjugate compounds encompass any pharmaceutically acceptable salts, esters, or salts of such esters, upon administration to an animal, including a human, is capable of providing (directly or indirectly) the biologically active metabolite or residue thereof. Accordingly, for example, the disclosure is also drawn to pharmaceutically acceptable salts of antisense oligonucleotide conjugate compounds, prodrugs, pharmaceutically acceptable salts of such prodrugs, and other bioequivalents. Suitable pharmaceutically acceptable salts include, but are not limited to, sodium and potassium salts. In some embodiments, the oligomer of the invention is a prodrug where the conjugate moiety is cleaved of the oligonucleotide once the prodrug is delivered to the site of action, in particular to a hepatocyte.

In a preferred embodiment the pharmaceutical compositions of the present invention are administered by a parenteral route including intravenous, intraarterial, subcutaneous, intraperitoneal or intramuscular injection or infusion; or intracranial, e.g., intrathecal or intraventricular, administration. In one embodiment the active oligomer or oligonucleotide conjugate is administered intravenously, this is particular relevant if the conjugate moiety is a sterol. In another embodiment the active oligomer or oligonucleotide conjugate is administered subcutaneously, this is particular relevant if the conjugate moiety is a N-acetylgalactosamine moiety.

### Applications

The oligomers, in particular the oligonucleotide conjugates of the invention may be utilized as research reagents for, for example, diagnostics, therapeutics and prophylaxis.

In research, such oligomers or oligonucleotide conjugates may be used to specifically inhibit the synthesis of ApoB protein (typically by degrading or inhibiting the mRNA and thereby prevent protein formation) in cells and experimental animals thereby facilitating functional analysis of the target or an appraisal of its usefulness as a target for therapeutic intervention.

In diagnostics the oligomers may be used to detect and quantitate APOB expression in cell and tissues by northern blotting, in-situ hybridisation or similar techniques.

For therapeutics, an animal or a human, suspected of having a disease or disorder, which can be treated by modulating the expression of APOB is treated by administering oligomeric compounds, in particular the oligonucleotide conjugates, in accordance with this invention. Further provided are methods of treating a mammal, such as treating a human, suspected of having or being prone to a disease or condition, associated with expression of APOB by administering a therapeutically or prophylactically effective amount of one or more of the oligomers or oligonucleotide conjugates or compositions of the invention. The oligomer, a conjugate or a pharmaceutical composition according to the invention is typically administered in an effective amount.

In a preferred embodiment the oligonucleotide conjugates of the invention are administered in an effective amount using a dose between 2.0 to 2.5 mg/kg, more preferably in a dose between 1.5 and 2.0 mg/kg, more preferably in a dose between 1.0 and 1.5 mg/kg, even more preferably in a dose between 0.5 and 1.0 mg/kg and most preferred in a dose between 0.1 and 0.5 mg/kg.

In a preferred embodiment the effective amount of the oligonucleotide conjugates of the invention reduces serum ApoB levels in an animal or human when compared to the ApoB serum level before treatment.

The invention also provides for the use of the compound or conjugate of the invention as described for the manufacture of a medicament for the treatment of a disorder as referred to herein, or for a method of the treatment of as a disorder as referred to herein.

The invention also provides for a method for treating a disorder as referred to herein said method comprising administering a compound according to the invention as herein described, and/or a conjugate according to the invention, and/or a pharmaceutical composition according to the invention to a patient in need thereof.

### Medical Indications

The oligomers, in particular the oligonucleotide conjugates, and other compositions according to the invention can be used for the treatment of conditions associated with over expression or expression of mutated version of ApoB.

The invention further provides use of a compound of the invention in the manufacture of a medicament for the treatment of a disease, disorder or condition as referred to herein.

Generally stated, one aspect of the invention is directed to a method of treating a mammal suffering from or susceptible to conditions associated with abnormal levels and/or activity of APOB, comprising administering to the mammal and therapeutically effective amount of an oligomer or oligonucleotide conjugate targeted to APOB. Preferably, the oligomer comprises one or more LNA units. The oligomer, the oligonucleotide conjugate or a pharmaceutical composition according to the invention is typically administered in an effective amount.

The disease or disorder, as referred to herein, may, in some embodiments be associated with a mutation in the APOB gene or a gene whose protein product is associated with or interacts with APOB. Therefore, in some embodiments, the target mRNA is a mutated form of the APOB sequence.

An interesting aspect of the invention is directed to the use of an oligomer (compound) as defined herein or a conjugate as defined herein for the preparation of a medicament for the treatment of a disease, disorder or condition as referred to herein.

The methods of the invention are preferably employed for treatment or prophylaxis against diseases caused by abnormal levels and/or activity of ApoB.

Alternatively stated, In some embodiments, the invention is furthermore directed to a method for treating abnormal levels and/or activity of ApoB, said method comprising administering a oligomer of the invention, or a conjugate of the invention or a pharmaceutical composition of the invention to a patient in need thereof.

The invention also relates to an oligomer, a composition or a conjugate as defined herein for use as a medicament.

The invention further relates to use of a compound, composition, or a conjugate as defined herein for the manufacture of a medicament for the treatment of abnormal levels and/or activity of APOB or expression of mutant forms of APOB (such as allelic variants, such as those associated with one of the diseases referred to herein).

Moreover, the invention relates to a method of treating a subject suffering from a disease or condition such as those referred to herein.

A patient who is in need of treatment is a patient suffering from or likely to suffer from the disease or disorder.

In some embodiments, the term ‘treatment’ as used herein refers to both treatment of an existing disease (e.g. a disease or disorder as herein referred to), or prevention of a disease, i.e. prophylaxis. It will therefore be recognised that treatment as referred to herein may, in some embodiments, be prophylactic.

In one embodiment, the invention relates to compounds or compositions comprising compounds for treatment of hypercholesterolemia and related disorders, or methods of treatment using such compounds or compositions for treating hypercholesterolemia and related disorders, wherein the term “related disorders” when referring to hypercholesterolemia refers to one or more of the conditions selected from the group consisting of: atherosclerosis, hyperlipidemia, hypercholesterolemia, familiar hypercholesterolemia e.g. gain of function mutations in APOB, HDL/LDL cholesterol imbalance, dyslipidemias, e.g., familial hyperlipidemia (FCHL), acquired hyperlipidemia, statin-resistant hypercholesterolemia, coronary artery disease (CAD), and coronary heart disease (CHD).

### Combination Treatments

In some embodiments the compound of the invention is for use in a combination treatment with another therapeutic agent. E.g. inhibitors of HMG CoA reductase, such as statins for example are widely used in the treatment of metabolic disease (see WO2009/043354, hereby incorporated by reference for examples of combination treatments). Combination treatments may be other cholesterol lowering compounds, such as may be selected from a compound is selected from the group consisting of bile salt sequestering resins (e.g., cholestyramine, colestipol, and colesevelam hydrochloride), HMGCoA-reductase inhibitors (e.g., lovastatin, cerivastatin, prevastatin, atorvastatin, simvastatin, and fluvastatin), nicotinic acid, fibric acid derivatives (e.g., clofibrate, gemfibrozil, fenofibrate, bezafibrate, and ciprofibrate), probucol, neomycin, dextrothyroxine, plant-stanol esters, cholesterol absorption inhibitors (e.g., ezetimibe), implitapide, inhibitors of bile acid transporters (apical sodium-dependent bile acid transporters), regulators of hepatic CYP7a, estrogen replacement therapeutics (e.g., tamoxifen), and anti-inflammatories (e.g., glucocorticoids). Combinations with statins may be particularly preferred.

### Specific Embodiments of the Invention

1. An antisense oligonucleotide conjugate comprising an oligomer with the oligonucleotide motif of SEQ ID NO 2 joined with a conjugate moiety (region C), where the conjugate moiety comprise a N-acetylgalactosamine moiety or a sterol moiety.
2. The antisense oligonucleotide conjugate according to embodiment 1, wherein the oligomer comprises at least 2 affinity enhancing nucleotide analogues.
3. The oligonucleotide conjugate according to embodiment 2, wherein the nucleotide analogues are sugar modified nucleotides, such as sugar modified nucleotides independently or dependently selected from the group consisting of: Locked Nucleic Acid (LNA) units; 2′-O-alkyl-RNA units, 2′-OMe-RNA units, 2′-amino-DNA units, and 2′-fluoro-DNA units.
4. The antisense oligonucleotide conjugate according to any one of embodiments 1 to 3, wherein the oligomer is a LNA containing oligomer.
5. The antisense oligonucleotide conjugate according to embodiment 3 or 4, wherein the LNA unit(s) is selected from the group consisting of beta-D-X-LNA or alpha-L-X-LNA (wherein X is oxy, amino or thio), ENA, cET, cMOE and 5′-Me-LNA.
6. The antisense oligonucleotide conjugate according to embodiment 5, wherein the LNA is beta-D-oxy-LNA.
7. The oligonucleotide conjugate according to any one of embodiments 1 to 6, wherein the oligomer is a gapmer.
8. The oligonucleotide conjugate according to embodiment 7, wherein the gapmer comprise a wing of 1 to 3 nucleotide analogues on each side (5′ and 3′) of a gap of 6 to 10 nucleotides.
9. The oligonucleotide conjugate according to embodiment 7 or 8, wherein the gapmer design is selected from the group consisting of 2-8-2, 2-7-3, 3-7-2 and 3-6-3.
10. The antisense oligonucleotide conjugate according to any one of the embodiments 1 to 9, wherein the oligomer comprises one or more nucleoside linkages selected from the group consisting of phosphorothioate, phosphorodithioate and boranophosphate.
11. The antisense oligonucleotide conjugate according to any one of embodiments 1 to 10, wherein the oligomer comprises or consist of phosphorothioate nucleoside linkages.
12. The antisense oligonucleotide conjugate according to any one of embodiments 1 to 11, wherein the oligomer corresponds to SEQ ID NO 27: 5′ GₛTₛtₛgₛaₛcₛaₛcₛtₛgₛTₛC 3′, wherein capital letters represent beta-D-oxy LNA, lower case letters represent DNA nucleosides, LNA cytosines are 5-methyl cytosine, and all internucleoside linkages are phosphorothioate indicated by s.
13. The antisense oligonucleotide conjugate according to any one of embodiments 1 to 12, wherein the oligomer is capable of down regulating the expression of ApoB in a cell which is expressing ApoB.
14. The antisense oligonucleotide conjugate according to embodiment 13, wherein the ApoB down regulation is in an animal or human.
15. The antisense oligonucleotide conjugate according to any one of embodiments 1 to 14, wherein the conjugate moiety comprises a sterol selected from cholesterol or tocopherol, such as those shown as Conj 5a and Conj 6a.
16. The antisense oligonucleotide conjugate according to any one of embodiments 1 to 15, wherein said conjugate moiety is joined to said oligomer, via a cleavable linker (B).
17. The antisense oligonucleotide conjugate according to embodiment 16, wherein the cleavable linker comprises a moiety selected from the group consisting of a peptide linker, a polypeptide linker, a lysine linker, or physiologically labile nucleotide linker.
18. The antisense oligonucleotide conjugate according to embodiment 16 or 17, wherein the bio cleavable linker comprises a physiologically labile nucleotide linker.
19. The antisense oligonucleotide conjugate according to embodiment 17 or 18, wherein the physiologically labile nucleotide linker is a phosphodiester nucleotide linkage comprising one or more contiguous DNA phosphodiester nucleotides, such as 1, 2, 3, 4, 5, or 6 DNA phosphodiester nucleotides which are contiguous with the 5′ or 3′ end of the contiguous sequence of the oligomer, and which may or may not form complementary base pairing with the ApoB target sequence.
20. The antisense oligonucleotide conjugate according to embodiment 19, wherein the phosphodiester nucleotide linkage (or biocleavable linker) comprises 1, 2 or 3 DNA phosphodiester nucleotides, such as two DNA phosphodiester nucleotides, such as a 5′ CA 3′ dinucleotide.
21. The antisense oligonucleotide conjugate according to embodiment 16 or 17, wherein the bio cleavable linker comprises a cleavable lysine linker, such as a di-lysine.
22. The antisense oligonucleotide conjugate according to any one of embodiments 1-14, or 16-21, wherein the conjugate moiety comprises one or more N-acetylgalactosamine moiety(s).
23. The antisense oligonucleotide conjugate according to any one of embodiments 1-14, or 16-21, wherein the conjugate moiety comprises 2 or 3 N-acetylgalactosamine moiety(s).
24. The antisense oligonucleotide conjugate according to any one of embodiments 1-14, or 16-23, wherein the conjugate moiety comprises a trivalent N-acetylgalactosamine cluster.
25. The antisense oligonucleotide conjugate according to any one of the preceding embodiments, wherein the oligonucleotide conjugate comprises a linker Y which covalently links the conjugate moiety to the oligomer.
26. The antisense oligomer conjugate according to embodiment 25, wherein the linker region Y comprises a fatty acid, such as a C6 to C12 linker, preferably a C6 linker.
27. The antisense oligonucleotide according to any one of embodiments 1-14 or 16 to 26, wherein the N-acetylgalactosamine moiety(s) comprises a flexible hydrophilic spacer.
28. The antisense oligonucleotide according to embodiment 27, wherein the hydrophilic spacer is a PEG spacer.
29. The antisense oligonucleotide conjugate according to any one of embodiments 1-14 or 16 to 28, wherein the conjugate moiety comprises three N-acetylgalactosamine moieties linked via a PEG spacer to a di-lysine.
30. The antisense oligonucleotide according to any one of embodiments 1-14, wherein the conjugate comprises a conjugate moiety selected from the group consisting of Conj1, Conj2, Conj3, Conj4, Conj1a, Conj2a, Conj3a and Conj4a.
31. The antisense oligonucleotide conjugate according to embodiment 30, wherein the conjugate moiety comprises Conj 2 or Conj2a, most preferably Conj2a.
32. The antisense oligonucleotide conjugate according to any one of the preceding embodiments, wherein the conjugate moiety (region C) does not comprise a pharmacokinetic modulator such as a fatty acid group of more than C6 in length.
33. The antisense oligomer according to embodiment 1, which consist of SEQ ID NO 28 or SEQ ID NO 31 or SEQ ID NO 29.
34. A pharmaceutical composition comprising the antisense oligonucleotide conjugate according to any one of embodiments 1 to 33, and a pharmaceutically acceptable diluent, carrier, salt or adjuvant.
35. The antisense oligonucleotide conjugate or pharmaceutical composition according to any one of embodiments 1 to 34, for use in reduction of serum ApoB levels in an animal or human.
36. The antisense oligonucleotide conjugate or pharmaceutical composition according to any one of embodiments 1 to 34, for use as a medicament.
37. The antisense oligonucleotide conjugate or pharmaceutical composition according to any one of embodiments 1 to 34, for use as a medicament such as for the treatment of acute coronary syndrome, or hypercholesterolemia or related disorder, such as a disorder selected from the group consisting of atherosclerosis, hyperlipidemia, hypercholesterolemia, HDL/LDL cholesterol imbalance, dyslipidemias, e.g., familial hyperlipidemia (FCHL), acquired hyperlipidemia, statin-resistant hypercholesterolemia, coronary artery disease (CAD), and coronary heart disease (CHD).
38. The antisense oligonucleotide conjugate or pharmaceutical composition according to any one of embodiments 1 to 34, for use in the treatment of acute coronary syndrome, or hypercholesterolemia or related disorder, such as a disorder selected from the group consisting of atherosclerosis, hyperlipidemia, hypercholesterolemia, HDL/LDL cholesterol imbalance, dyslipidemias, e.g., familial hyperlipidemia (FCHL), acquired hyperlipidemia, statin-resistant hypercholesterolemia, coronary artery disease (CAD), and coronary heart disease (CHD).
39. The use of an antisense oligonucleotide conjugate or pharmaceutical composition according to any one of the embodiments 1 to 34, for the manufacture of a medicament for the treatment of acute coronary syndrome, or hypercholesterolemia or a related disorder, such as a disorder selected from the group consisting of atherosclerosis, hyperlipidemia, hypercholesterolemia, HDL/LDL cholesterol imbalance, dyslipidemias, e.g., familial hyperlipidemia (FCHL), acquired hyperlipidemia, statin-resistant hypercholesterolemia, coronary artery disease (CAD), and coronary heart disease (CHD).
40. A method of treating acute coronary syndrome, or hypercholesterolemia or a related disorder, such as a disorder selected from the group consisting atherosclerosis, hyperlipidemia, hypercholesterolemia, HDL/LDL cholesterol imbalance, dyslipidemias, e.g., familial hyperlipidemia (FCHL), acquired hyperlipidemia, statin-resistant hypercholesterolemia, coronary artery disease (CAD), and coronary heart disease (CHD), said method comprising administering an effective amount of an antisense oligonucleotide conjugate or pharmaceutical composition according to any one of the embodiments 1 to 34, to a patient suffering from, or likely to suffer from hypercholesterolemia or a related disorder.
41. An in vivo or in vitro method for the inhibition of ApoB in a cell which is expressing ApoB, said method comprising administering an oligonucleotide conjugate or pharmaceutical composition according to any one of the embodiments 1 to 34 to said cell so as to inhibit ApoB in said cell.

### EXAMPLES

### Oligonucleotide ApoB Targeting Compounds

The tables below show the oligonucleotide sequence motifs complementary to the ApoB gene (NCBI accession number NM_000384 and SEQ ID NO: 32) and oligonucleotide designs used in the examples.

TABLE 2
Oligonucleotide sequence motifs

| | | Position on |
|---|---|---|
| SEQ | Sequence motif | the ApoB gene |
| ID NO | (5′-3′) | SEQ ID NO: 32 |
| 1 | GCATTGGTATTCA | 10177-10189 |
| 2 | GTTGACACTGTC | 2265-2277 |

TABLE 3
ApoB Targeting Compounds
with cholesterol conjugates

| | Oligo Sequence | Cleavable | |
|---|---|---|---|
| SEQ | (5′-3′) | Linker | Region C- |
| ID NO | (Region A) | (Region B) | Conjugate |
| 3 | GCattggtatTCA | no | no |
| 4 | GCattggtatTCA | no | Cholesterol |
| 5 | GCattggtatTCA | SS | Cholesterol |
| 6 | GCattggtatTCA | 3PO-DNA (5′tca3′) | Cholesterol |
| 7 | GCattggtatTCA | 2PO-DNA (5′ca3′) | Cholesterol |
| 8 | GCattggtatTCA | 1PO-DNA (5′a3′) | Cholesterol |

The compounds are illustrated in FIG. 12B

TABLE 4
ApoB Targeting Compounds
with FAM label conjugates

| | Oligo Sequence | | |
|---|---|---|---|
| SEQ | (5′-3′) | Cleavable | Conjugate |
| ID NO | (Region A) | linker (B) | (C) |
| 9 | GCattggtatTCA | 3PO-DNA (5′tca3′) | FAM |
| 10 | GCattggtatTCA | 2PO-DNA (5′ca3′) | FAM |
| 11 | GCattggtatTCA | 1PO-DNA (5′a3′) | FAM |
| 12 | GCattggtatTCA | 3PO-DNA (5′gac3′) | FAM |
| 13 | GCattggtatTCA | no | FAM |

The compounds are illustrated in FIG. 12C

TABLE 5
ApoB Targeting Compounds with
different conjugates and linkers

| | Oligo Sequence | | |
|---|---|---|---|
| SEQ | (5′-3′) | Cleavable | |
| ID NO | (Region A) | Linker (B) | Conjugate |
| 14 | GCattggtatTCA | no | Folic acid |
| 15 | GCattggtatTCA | SS | Folic acid |
| 16 | GCattggtatTCA | 2PO-DNA | Folic acid |
| | | (5′ca3′) | |
| 17 | GCattggtatTCA | no | monoGalNAc |
| 18 | GCattggtatTCA | SS | monoGalNAc |
| 19 | GCattggtatTCA | 2PO-DNA | monoGalNAc |
| | | (5′ca3′) | |
| 20 | GCattggtatTCA | | GalNAc cluster |
| | | | Conj2a |
| 21 | GCattggtatTCA | no | FAM |
| 22 | GCattggtatTCA | SS | FAM |
| 23 | GCattggtatTCA | 2PO-DNA | FAM |
| | | (5′ca3′) | |
| 24 | GCattggtatTCA | no | Tocopherol |
| 25 | GCattggtatTCA | SS | Tocopherol |
| 26 | GCattggtatTCA | 2PO-DNA | Tocopherol |
| | | (5′ca3′) | |
| 30 | GCattggtatTCA | | GalNAc cluster |
| | | | Conj1a |

The compounds are illustrated in FIG. 12D

TABLE 6
ApoB Targeting Compounds with
different conjugates and linkers

| | Oligo Sequence | | |
|---|---|---|---|
| SEQ | (5′-3′) | Cleavable | |
| ID NO | (Region A) | Linker (B) | Conjugate |
| 27 | GttgacactgTC | no | no |
| 28 | GttgacactgTC | 2PO-DNA | Cholesterol |
| | | (5′ca3′) | |
| 29 | GttgacactgTC | | GalNAc cluster |
| | | | Conj2a |
| 31 | GttgacactgTC | | GalNAc cluster |
| | | | Conj1a |

The compounds are illustrated in FIG. 12E

In the table 3 to 6 Capital letters are LNA nucleosides (such as beta-D-oxy LNA), lower case letters are DNA nucleosides. LNA cytosines are 5-methyl cytosine. Internucleoside linkages in the oligonucleotide (oligo) sequence are phosphorothioate internucleoside linkages.

### Mouse Experiments

Unless otherwise specified, the mouse experiments may be performed as follows:

### Dose Administration and Sampling:

7-10 week old C57B16-N mice were used, animals were age and sex matched (females for study 1, 2 and 4, males in study 3). Compounds were injected i.v. into the tail vein. For intermediate serum sampling, 2-3 drops of blood were collected by puncture of the vena facialis, final bleeds were taken from the vena cava inferior. Serum was collected in gel-containing serum-separation tubes (Greiner) and kept frozen until analysis.

C57BL6 mice were dosed i.v. with a single dose of 1 mg/kg antisense oligomer (ASO) (or amount shown) formulated in saline or saline alone according to the information shown. Animals were sacrificed at e.g. day 4 or 7 (or time shown) after dosing and liver and kidney were sampled.

RNA isolation and mRNA analysis: mRNA analysis from tissue was performed using the Qantigene mRNA quantification kit (“bDNA-assay”, Panomics/Affimetrix), following the manufacturers protocol. For tissue lysates, 50-80 mg of tissue was lysed by sonication in 1 ml lysis-buffer containing Proteinase K. Lysates were used directly for bDNA-assay without RNA extraction. Probe sets for the target and GAPDH were obtained custom designed from Panomics. For analysis, luminescence units obtained for target genes were normalized to the housekeeper GAPDH.

Serum analysis for ALT, AST and cholesterol was performed on the “Cobas INTEGRA 400 plus” clinical chemistry platform (Roche Diagnostics), using 10 μl of serum.

For oligonucleotide quantification, a fluorescently-labeled PNA probe is hybridized to the oligonucleotide of interest in the tissue lysate. The same lysates are used as for bDNA-assays, just with exactly weighted amounts of tissue. The heteroduplex is quantified using AEX-HPLC and fluorescent detection.

### Example 1

### Synthesis of Compounds

Oligonucleotides were synthesized on uridine universal supports using the phosphoramidite approach on an Oligomaker 48 at 1 μmol scale. At the end of the synthesis, the oligonucleotides were cleaved from the solid support using aqueous ammonia for 5-16 hours at 60° C. The oligonucleotides were purified by reverse phase HPLC (RP-HPLC) or by solid phase extractions and characterized by UPLC, and the molecular mass was further confirmed by ESI-MS. See below for more details.

Elongation of the Oligonucleotide:

The coupling of β-cyanoethyl-phosphoramidites (DNA-A(Bz), DNA-G(ibu), DNA-C(Bz), DNA-T, LNA-5-methyl-C(Bz), LNA-A(Bz), LNA-G(dmf), LNA-T or C6-S-S linker) is performed by using a solution of 0.1 M of the 5′-O-DMT-protected amidite in acetonitrile and DCI (4,5-dicyanoimidazole) in acetonitrile (0.25 M) as activator. For the final cycle a commercially available C6-linked cholesterol phosphoramidite was used at 0.1 M in DCM. Thiolation for introduction of phosphorthioate linkages is carried out by using xanthane hydride (0.01 M in acetonitrile/pyridine 9:1). Phosphordiester linkages are introduced using 0.02 M iodine in THF/Pyridine/water 7:2:1. The rest of the reagents are the ones typically used for oligonucleotide synthesis. For post solid phase synthesis conjugation a commercially available C6 aminolinker phorphoramidite was used in the last cycle of the solid phase synthesis and after deprotection and cleavage from the solid support the aminolinked deprotected oligonucleotide was isolated. The conjugates were introduced via activation of the functional group using standard synthesis methods.

Purification by RP-HPLC:

The crude compounds were purified by preparative RP-HPLC on a Phenomenex Jupiter C18 10μ 150×10 mm column. 0.1 M ammonium acetate pH 8 and acetonitrile was used as buffers at a flow rate of 5 mL/min. The collected fractions were lyophilized to give the purified compound typically as a white solid.

### ABBREVIATIONS

DCI: 4,5-Dicyanoimidazole

DCM: Dichloromethane

DMF: Dimethylformamide

DMT: 4,4′-Dimethoxytrityl

THF: Tetrahydrofurane

Bz: Benzoyl

Ibu: Isobutyryl

RP-HPLC: Reverse phase high performance liquid chromatography

### Example 2

### Knock Down of ApoB mRNA with Cholesterol-Conjugates In Vivo

C57BL6/J mice were injected with a single dose saline or 1 mg/kg unconjugated LNA-antisense oligonucleotide (SEQ ID NO 3) or equimolar amounts of the LNA antisense oligonucleotide conjugated to Cholesterol with different linkers (see table 7 below) and sacrificed at days 1-10 according to the table below. RNA was isolated from liver and kidney and subjected to qPCR with ApoB specific primers and probe to analyses for ApoB mRNA knockdown.

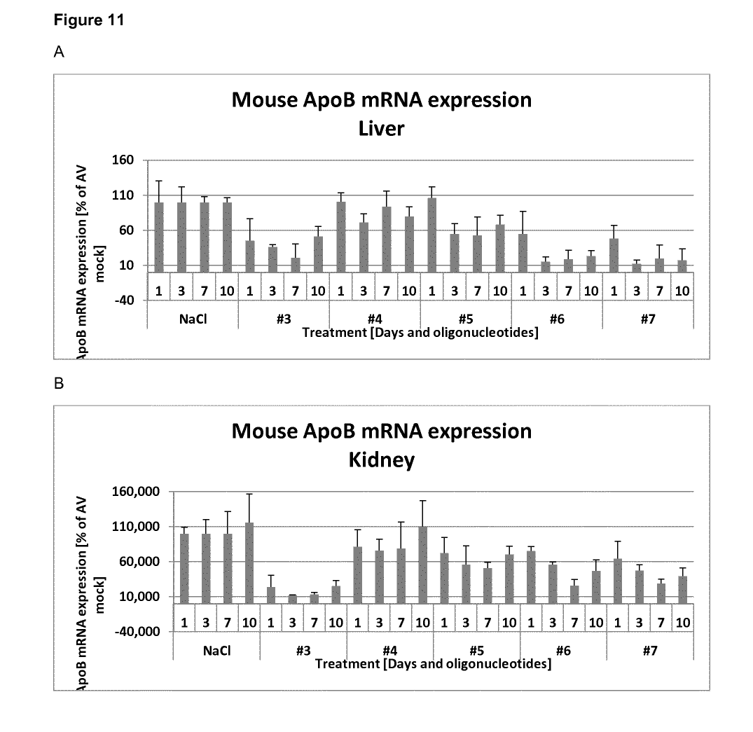

TABLE 7
ApoB Targeting Compounds with
different conjugates and linkers

| SEQ | | |
|---|---|---|
| ID | Compound Sequence | Comment |
| 3 | 5′- [symbol] aₛ tₛ tₛ gₛ gₛ tₛ aₛ | No conjugate |
| | tₛ [symbol] -3′ | FIG. 11 #3 |
| 4 | 5′-Chol_C6 [symbol] aₛ tₛ tₛ gₛ | Chol-Compound |
| | gₛ tₛ aₛ tₛ [symbol] -3′ | FIG. 11 #4 |
| 5 | 5′-Chol_C6 C6SSC6 [symbol] aₛ | Chol-SS-#1 |
| | tₛ tₛ gₛ gₛ tₛ aₛ tₛ [symbol] -3′ | FIG. 12 #5 |
| 6 | 5′-Chol_C6 t c a [symbol] aₛ tₛ | Chol-3PO-#1 |
| | tₛ gₛ gₛ tₛ aₛ tₛ [symbol] -3′ | FIG. 11 #6 |
| 7 | 5′-Chol_C6 c a [symbol] aₛ tₛ | Chol-2PO-#1 |
| | tₛ gₛ gₛ tₛ aₛ tₛ [symbol] -3′ | FIG. 11 #7 |

Uppercase letters denote beta-D-oxy-LNA monomers; lowercase letters denote DNA monomers the subscript “s” denotes a phosphorothioate linkage the superscript “m” denotes a beta-D-oxy-LNA monomer containing a 5-methylcytosine base; the superscript “o” denotes Oxy-LNA.

### Materials and Methods:

Experimental Design:

| | | | | | Compound | Conc. at | | |
|---|---|---|---|---|---|---|---|---|
| | Gr. | Animal | No of | Animal strain/ | Dose level | dose vol. | Body | |
| | no. | ID no. | animals | gender/feed | per day | 10 ml/kg | weight | Sacrifice |
| A | 1 | 1-4 | 4 | C57BL/6J- | NaCl 0.9% | — | Day −1, 7 | Day 10 |
| | | | | ♀-Chow | | | and 10 | |
| | 2 | 5-8 | 4 | C57BL/6J- | SEQ ID 3 | 0.1 mg/ml | Day −1, 7 | Day 10 |
| | | | | ♀-Chow | 1 mg/kg | | and 10 | |
| | 3 | 9-12 | 4 | C57BL/6J- | SEQ ID 4 | 0.12 mg/ml | Day −1, 7 | Day 10 |
| | | | | ♀-Chow | 1.2 mg/kg | | and 10 | |
| | 4 | 13-16 | 4 | C57BL/6J- | SEQ ID 5 | 0.12 mg/ml | Day −1, 7 | Day 10 |
| | | | | ♀-Chow | 1.2 mg/kg | | and 10 | |
| | 5 | 17-20 | 4 | C57BL/6J- | SEQ ID 6 | 0.13 mg/ml | Day −1, 7 | Day 10 |
| | | | | ♀-Chow | 1.3 mg/kg | | and 10 | |
| | 6 | 21-24 | 4 | C57BL/6J- | SEQ ID 7 | 0.13 mg/ml | Day −1, 7 | Day 10 |
| | | | | ♀-Chow | 1.3 mg/kg | | and 10 | |
| B | 7 | 25-28 | 4 | C57BL/6J- | NaCl 0.9% | — | Day −1, 7 | Day 7 |
| | | | | ♀-Chow | | | | |
| | 8 | 29-32 | 4 | C57BL/6J- | SEQ ID 3 | 0.1 mg/ml | Day −1, 7 | Day 7 |
| | | | | ♀-Chow | 1 mg/kg | | | |
| | 9 | 33-36 | 4 | C57BL/6J- | SEQ ID 4 | 0.12 mg/ml | Day −1, 7 | Day 7 |
| | | | | ♀-Chow | 1.2 mg/kg | | | |
| | 10 | 37-40 | 4 | C57BL/6J- | SEQ ID 5 | 0.12 mg/ml | Day −1, 7 | Day 7 |
| | | | | ♀-Chow | 1.2 mg/kg | | | |
| | 11 | 41-44 | 4 | C57BL/6J- | SEQ ID 6 | 0.13 mg/ml | Day −1, 7 | Day 7 |
| | | | | ♀-Chow | 1.3 mg/kg | | | |
| | 12 | 45-48 | 4 | C57BL/6J- | SEQ ID 7 | 0.13 mg/ml | Day −1, 7 | Day 7 |
| | | | | ♀-Chow | 1.3 mg/kg | | | |
| C | 13 | 49-52 | 4 | C57BL/6J- | NaCl 0.9% | — | Day 0, 3 | Day 3 |
| | | | | ♀-Chow | | | | |
| | 14 | 53-56 | 4 | C57BL/6J- | SEQ ID 3 | 0.1 mg/ml | Day 0, 3 | Day 3 |
| | | | | ♀-Chow | 1 mg/kg | | | |
| | 15 | 57-60 | 4 | C57BL/6J- | SEQ ID 4 | 0.12 mg/ml | Day 0, 3 | Day 3 |
| | | | | ♀-Chow | 1.2 mg/kg | | | |
| | 16 | 61-64 | 4 | C57BL/6J- | SEQ ID 5 | 0.12 mg/ml | Day 0, 3 | Day 3 |
| | | | | ♀-Chow | 1.2 mg/kg | | | |
| | 17 | 65-68 | 4 | C57BL/6J- | SEQ ID 6 | 0.13 mg/ml | Day 0, 3 | Day 3 |
| | | | | ♀-Chow | 1.3 mg/kg | | | |
| | 18 | 69-72 | 4 | C57BL/6J- | SEQ ID 7 | 0.13 mg/ml | Day 0, 3 | Day 3 |
| | | | | ♀-Chow | 1.3 mg/kg | | | |
| D | 19 | 73-76 | 4 | C57BL/6J- | NaCl 0.9% | — | Day −1, 1 | Day 1 |
| | | | | ♀-Chow | | | | |
| | 20 | 77-80 | 4 | C57BL/6J- | SEQ ID 3 | 0.1 mg/ml | Day −1, 1 | Day 1 |
| | | | | ♀-Chow | 1 mg/kg | | | |
| | 21 | 81-84 | 4 | C57BL/6J- | SEQ ID 4 | 0.12 mg/ml | Day −1, 1 | Day 1 |
| | | | | ♀-Chow | 1.2 mg/kg | | | |
| | 22 | 85-88 | 4 | C57BL/6J- | SEQ ID 5 | 0.12 mg/ml | Day −1, 1 | Day 1 |
| | | | | ♀-Chow | 1.2 mg/kg | | | |
| | 23 | 89-92 | 4 | C57BL/6J- | SEQ ID 6 | 0.13 mg/ml | Day −1, 1 | Day 1 |
| | | | | ♀-Chow | 1.3 mg/kg | | | |
| | 24 | 93-96 | 4 | C57BL/6J- | SEQ ID 7 | 0.13 mg/ml | Day −1, 1 | Day 1 |
| | | | | ♀-Chow | 1.3 mg/kg | | | |

Dose Administration.

C57BL/6JBorn female animals, app. 20 g at arrival, were dosed with 10 ml per kg BW (according to day 0 bodyweight) i.v. of the compound formulated in saline or saline alone according to the above table.

Sampling of Liver and Kidney Tissue.

The animals were anaesthetized with 70% CO₂-30% O₂ and sacrificed by cervical dislocation according to the table above. One half of the large liver lobe and one kidney were minced and submerged in RNAlater.

Total RNA Isolation and First Strand Synthesis.

Total RNA was extracted from maximum 30 mg of tissue homogenized by bead-milling in the presence of RLT-Lysis buffer using the Qiagen RNeasy kit (Qiagen cat. no. 74106) according to the manufacturer's instructions. First strand synthesis was performed using Reverse Transcriptase reagents from Ambion according to the manufacturer's instructions.

For each sample 0.5 μg total RNA was adjusted to (10.8 μl) with RNase free H₂O and mixed with 2 μl random decamers (50 μM) and 4 μl dNTP mix (2.5 mM each dNTP) and heated to 70° C. for 3 min after which the samples were rapidly cooled on ice. 2 μl 10× Buffer RT, 1 μl MMLV Reverse Transcriptase (100 U/μl) and 0.25 μl RNase inhibitor (10 U/μl) were added to each sample, followed by incubation at 42° C. for 60 min, heat inactivation of the enzyme at 95° C. for 10 min and then the sample was cooled to 4° C. cDNA samples were diluted 1: 5 and subjected to RT-QPCR using Taqman Fast Universal PCR Master Mix 2x (Applied Biosystems Cat #4364103) and Taqman gene expression assay (mApoB, Mn01545150_m1 and mGAPDH #4352339E) following the manufacturers protocol and processed in an Applied Biosystems RT-qPCR instrument (7500/7900 or ViiA7) in fast mode.

The results are shown in FIG. 11.

Conclusions:

Cholesterol conjugated to an ApoB LNA antisense oligonucleotide with a linker composed of 2 or 3 DNA with phosphodiester backbone (SEQ ID NO 6 and 7) showed a preference for liver specific knock down of ApoB (FIG. 11). In conclusion the cholesterol conjugated oligonucleotides with a cleavable linker (SEQ ID NO 6 and 7) increased the efficacy and duration of ApoB mRNA knock down in liver tissue compared to the unconjugated compound (SEQ ID NO 3), as well as compared to Cholesterol conjugates with stable linker (SEQ ID NO 4) and with disulphide linker (SEQ ID NO 5) and concomitant less knock down activity of SEQ ID NO 6 and SEQ ID NO 7 in kidney tissue.

### Example 3

### In Vivo Silencing of ApoB mRNA with Different Conjugates

To explore the impact of different conjugation moieties and linkers on the activity of an ApoB compound, SEQ ID NO 3 was conjugated to either monoGalNAc, Folic acid, FAM or Tocopherol using a non-cleavable linker or biocleavable linker (Dithio (SS) or 2 DNA nucleotides with Phosphodiester backbone (PO)). Additionally the monoGalNAc was compared to a GalNAc cluster (Conjugate 2a). See Table 5 for more construct details. C57BL6 In mice were treated i.v. with saline control or with a single dose of 1 or 0.25 mg/kg of ASO conjugates. After 7 days the animals were sacrificed and RNA was isolated from liver and kidney samples and analysed for ApoB mRNA expression

Materials and Methods:

Experimental Design:

| | | Animal | | | | | |
|---|---|---|---|---|---|---|---|
| | Animals | strain/ | Com- | | | | |
| Gr. | per | gender/ | pound | Dose | Adm. | Dosing | Sacrifice |
| no. | group | feed | Seq ID # | mg/kg | Route | Day | Day |
| 1 | 5 | C57BL6 | 3 | 1 | i.v. | 0 | 7 |
| | | ♀-Chow | | | | | |
| 2 | 5 | C57BL6 | 14 | 1 | i.v. | 0 | 7 |
| | | ♀-Chow | | | | | |
| 3 | 5 | C57BL6 | 15 | 1 | i.v. | 0 | 7 |
| | | ♀-Chow | | | | | |
| 4 | 5 | C57BL6 | 16 | 1 | i.v.. | 0 | 7 |
| | | ♀-Chow | | | | | |
| 5 | 5 | C57BL6 | 17 | 1 | i.v. | 0 | 7 |
| | | ♀-Chow | | | | | |
| 6 | 5 | C57BL6 | 18 | 1 | i.v. | 0 | 7 |
| | | ♀-Chow | | | | | |
| 7 | 5 | C57BL6 | 19 | 1 | i.v. | 0 | 7 |
| | | ♀-Chow | | | | | |
| 8 | 5 | C57BL6 | 19 | 0.25 | i.v. | 0 | 7 |
| | | ♀-Chow | | | | | |
| 9 | 5 | C57BL6 | 20 | 0.25 | i.v. | 0 | 7 |
| | | ♀-Chow | | | | | |
| 10 | 5 | C57BL6 | NaCl | | i.v. | 0 | 7 |
| | | ♀-Chow | 0.9% | | | | |
| 1 | 5 | C57BL6 | 3 | 1 | i.v. | 0 | 7 |
| | | ♀-Chow | | | | | |
| 2 | 5 | C57BL6 | 21 | 1 | i.v. | 0 | 7 |
| | | ♀-Chow | | | | | |
| 3 | 5 | C57BL6 | 22 | 1 | i.v. | 0 | 7 |
| | | ♀-Chow | | | | | |
| 4 | 5 | C57BL6 | 23 | 1 | i.v. | 0 | 7 |
| | | ♀-Chow | | | | | |
| 5 | 5 | C57BL6 | 24 | 1 | i.v. | 0 | 7 |
| | | ♀-Chow | | | | | |
| 6 | 5 | C57BL6 | 25 | 1 | i.v. | 0 | 7 |
| | | ♀-Chow | | | | | |
| 7 | 5 | C57BL6 | 26 | 1 | i.v. | 0 | 7 |
| | | ♀-Chow | | | | | |
| 8 | 5 | C57BL6 | NaCl | 1 | i.v. | 0 | 7 |
| | | ♀-Chow | 0.9% | | | | |

Dose Administration and Sampling.

C57BL6 mice were dosed i.v. with a single dose of 1 mg/kg or 0.25 mg/kg ASO formulated in saline or saline alone according to the above table. Animals were sacrificed at day7 after dosing and liver and kidney were sampled. RNA isolation and mRNA analysis. Total RNA was extracted from liver and kidney samples and ApoB mRNA levels were analysed using a branched DNA assay

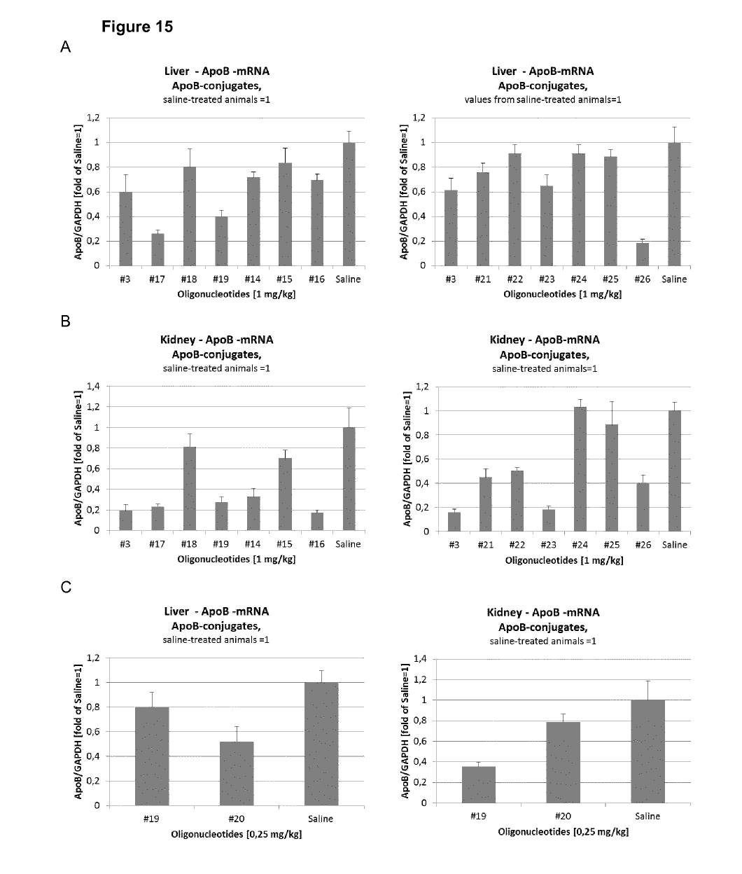

The results are shown in FIG. 15.

Conclusions:

Tocopherol conjugated to the ApoB compound with a DNA/PO-linker (SEQ ID NO 26) increased ApoB knock down in the liver compared to the unconjugated ApoB compound (SEQ ID NO 3) while decreasing activity in kidney (compare FIGS. 15 A and B). This points towards an ability of the Tocopherol to redirect the ApoB compound from kidney to liver. The non-cleavable (SEQ ID NO 24) and SS-linked (SEQ ID NO 25) Tocopherol conjugates were inactive in both tissues. Mono-GalNAc conjugates with a non-cleavable (SEQ ID NO 17) and with bio-cleavable DNA/PO linker (SEQ ID NO 19) show a tendency to preserve the activity of the unconjugated compound (SEQ ID NO 3) in kidney while improving activity in the Liver. Introduction of a SS-linker decreased activity in both tissues (compare FIGS. 15A and B). Conjugation of different GalNAc conjugates e.g. mono GalNAcPO (SEQ ID NO 19) and a GalNAc cluster (SEQ ID NO 20) also allows fine tuning of the compound activity with focus on either liver or kidney (FIG. 15C). Folic acid and FAM conjugates with the cleavable DNA/PO-linker (SEQ ID NO: 16 and 23) behave comparable to the unconjugated compound (SEQ ID NO: 3). Here as well the introduction of a non-cleavable (SEQ ID NO 14 and 21) or SS-linker (SEQ ID NO 15 and 22) decreases compound activity in both tissues (compare FIGS. 15a and 15b).

### Example 4A

### Effect of Non-Conjugated Anti-ApoB LNA Compounds in Non-Human Primates

The following example compares data from two different monkey studies with the purpose to compare the effectiveness of the non-conjugated anti-ApoB LNA compounds in relation to each other.

SEQ ID NO 3 and SEQ ID NO 27 have previously been tested in multiple dose studies in cynomolgus monkeys. Data from the study on SEQ ID NO 3 has previously been published (Straarup et al, Nucleic Acids Research, 2010, Vol. 38, pages 7100-7110).

The study of SEQ ID 27 was performed at Bridge Laboratories 32 Kexue Yuan Road, Zhongguancun Life Science Park, Changping District, Beijing 102206, People's Republic of China. The objective of this study was to evaluate the toxicity of SEQ ID NO 27 in male and female cynomolgus monkeys when administered for 2 weeks or 13 weeks, and to assess the reversibility, progression, and/or potential delayed effects during 6-week and 8-week observation periods following the 2- and 13-week treatment periods, respectively. Age at first day of dosing was 2.0-4.0 years, weight at first day of dosing 2.0-4.0 kg. SEQ ID NO 27 was administered at 1, 4, 8, or 24 mg/kg/injection. Animals were injected at days 1, 6, 11, 16, 23, 30, 37, 44, 51, 58, 65, 72, 79, and 86.

The effect on LDL-C reduction obtained in the two studies was compared at similar time points as shown in Table 8 below.

TABLE 8

| | | | Total dose | LDL-C (as % of |
|---|---|---|---|---|
| | Dosing before LDL-C | | before LDL-C | control at the |
| Compound | analysis | Dose level | analysis | same time point) |
| SEQ ID NO 3 | Injected day 1, 7 | 2 mg/kg | 2 × 2 mg/kg | 60 ± 17% day 14 |
| SEQ ID NO 27 | Injected day 1, 6, 11, 16 | 4 mg/kg | 4 × 4 mg/kg | 60 ± 11% day 17 |

Both compounds (SEQ ID NO 3 and SEQ ID NO 27) reduced LDL-C to 60% of LDL-C in saline (control) animals in respective study, but the effect was achieved at very different doses. SEQ ID NO 3 demonstrated significantly higher potency than for SEQ ID NO 27 when administered to male and female cynomolgus monkeys, in that two doses of 2 mg/kg (total dose 2×2 mg/kg) of SEQ ID NO 3 had the same effect on the final pharmacology end point (lowering of LDL-C) as four doses of 4 mg/kg (total dose 4×4 mg/kg) of SEQ ID NO 27.

### Example 4B

### Non-Human Primate (NHP) Studies

The primary objective for this study was to investigate selected lipid markers over 7 weeks after a single slow intravenous bolus injection of anti-ApoB LNA conjugated compounds to cynomolgus monkeys and assess the potential toxicity of compounds in monkey. The compounds used in this study were SEQ ID NO 7, 20, 28 & 29, prepared in sterile saline (0.9%) at an initial concentration of 0.625 and 2.5 mg/ml).

Female monkeys of at least 24 months old were used, and given free access to tap water and 180 g of OWM(E) SQC SHORT expanded diet (Dietex France, SDS, Saint Gratien, France) was distributed daily per animal. In addition, fruit or vegetables was given daily to each animal. The animals were acclimated to the study conditions for a period of at least 14 days before the beginning of the treatment period. During this period, pre-treatment investigations were performed. The animals were dosed i.v. at a dose of, 1 mg/kg. The dose volume was 0.4 mL/kg. Two animals were used per group. After three weeks, the data were analyzed and a second group of animals using a higher or lower dosing regimen was initiated—preliminary dose setting was 2.5 mg/kg, or lower than that based on the first data set.

The dose formulations were administered once on Day 1. Animals were observed for a period of 7 weeks following treatment. Day 1 corresponds to the first day of the treatment period. Clinical observations, body weight and food intake (per group) was recorded prior to and during the study.

Blood was sampled and analyses performed at the following time points:

| Study Day | Parameters |
|---|---|
| −8 | RCP, L, Apo-B, OA |
| −1 | L, Apo-B, PK, OA |
| 1 | Dosing |
| 4 | LSB, L, Apo-B, OA |
| 8 | LSB, L, Apo-B, PK, OA |
| 15 | RCP, L, Apo-B, PK, OA |
| 22 | LSB, L, Apo-B, PK, OA |
| 29 | L, Apo-B, PK, OA |
| 36 | LSB, L, Apo-B, PK, OA |
| 43 | L, PK, Apo-B, PK, OA |
| 50 | RCP, L, Apo-B, PK, OA |

RCP = routine clinical pathology,
LSB = liver safety biochemistry,
PK = pharmacokinetics,
OA = other analysis,
L = Lipids.

Blood biochemistry: The following parameters was determined for all surviving animals at the occasions indicated below:
full biochemistry panel (complete list below)—on Days −8, 15 and 50,
liver Safety (ASAT, ALP, ALAT, TBIL and GGT only)—on Days 4, 8, 22 and 36,
lipid profile (Total cholesterol, HDL-C, LDL-C and Triglycerides) and Apo-B only—on Days −1, 4, 8, 22, 29, 36, and 43.

Blood (approximately 1.0 mL) was taken into lithium heparin tubes (using the ADVIA 1650 blood biochemistry analyser): Apo-B, sodium, potassium, chloride, calcium, inorganic phosphorus, glucose, HDL-C, LDL-C, urea, creatinine, total bilirubin (TBIL), total cholesterol, triglycerides, alkaline phosphatase (ALP), alanine aminotransferase (ALAT), aspartate aminotransferase (ASAT), creatine kinase, gamma-glutamyl transferase (GGT), lactate dehydrogenase, total protein, albumin, albumin/globulin ratio.

Analysis of Blood:

Blood samples for ApoB analysis were collected on Days −8, −1, 4, 8, 15, 22, 29, 36, 43 and 50. Blood was centrifuged at 1000 g for 10 minutes under refrigerated conditions (set to maintain+4° C.). The serum was transferred into 3 individual tubes and stored at −80° C. until analysis.

Other Analysis:

WO2010142805 provides the methods for the following analysis: qPCR, ApoB mRNA analysis (hereby incorporated by reference). Other analysis includes ApoB protein ELISA, serum Lp(a) analysis with ELISA (Mercodia No. 10-1106-01), tissue and serum oligonucleotide analysis (drug content), Extraction of samples, standard- and QC-samples, Oligonucleotide content determination by ELISA.

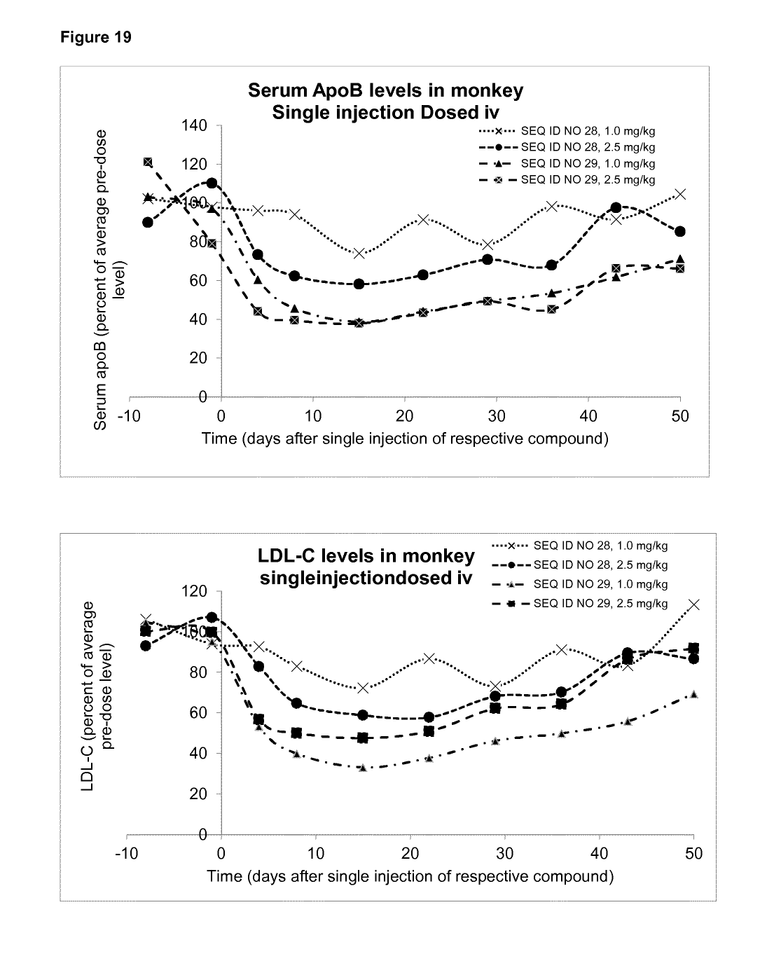

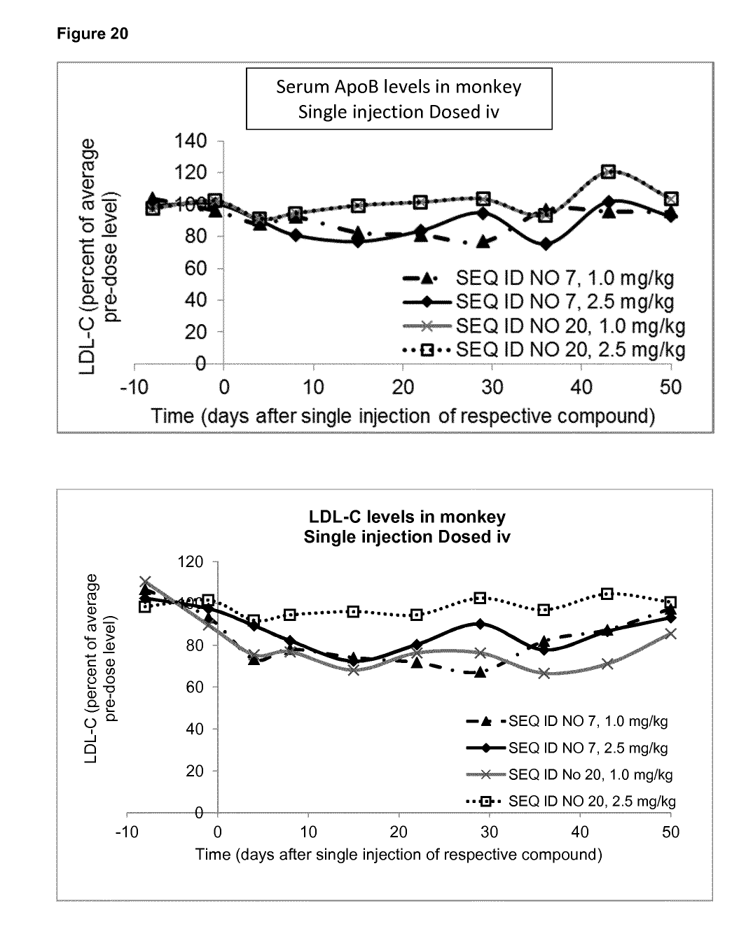

The data for SEQ ID NO 27 conjugate compounds (SEQ ID NO: 28 and 29) is shown in FIG. 19, and the data for SEQ ID NO 3 conjugates (SEQ ID NO 7 and 20) are shown in FIG. 20. Notably, in the NHP study the conjugated compounds of SEQ ID NO 3 (SEQ ID NO 7 and 20) did not result in a notable decrease in ApoB or LDL cholesterol at the doses used (FIG. 20), despite the parent compound (SEQ ID NO 3) being more potent than the parent compound of SEQ ID NO 27 as described in Example 4A. There was no indication of hepatotoxicity or nephrotoxicity with any of the ApoB targeting compounds. Notably, the SEQ ID NO 27-GalNAc compound (SEQ ID NO 29) gave a rapid and highly effective down regulation of ApoB and LDL which was maintained over an extensive time period (entire length of the study). This illustrated that the GalNAc conjugated SEQ ID NO: 27 compound (SEQ ID NO: 29) was more effective, both in terms of a rapid initial knock-down and long duration, than the cholesterol conjugate (FIG. 19), although the cholesterol conjugated SEQ ID NO: 27 (SEQ ID NO: 28) also had quite good effects at the 2.5 mg/kg dose. This is an indication that the GalNAc compound may be dosed comparatively infrequently and at a lower dosage, as compared to both the unconjugated parent compound, and compounds using alternative conjugation technology.

### Example 5

### Liver and Kidney Toxicity Assessment in Rat

Compounds of the invention can be evaluated for their toxicity profile in rodents, such as in mice or rats. By way of example the following protocol may be used: Wistar Han Crl:WI(Han) are used at an age of approximately 8 weeks old. At this age, the males should weigh approximately 250 g. All animals have free access to SSNIFF R/M-H pelleted maintenance diet (SSNIFF Spezialdiaten GmbH, Soest, Germany) and to tap water (filtered with a 0.22 μm filter) contained in bottles. The dose level of 10 and 40 mg/kg/dose is used (sub-cutaneous administration) and dosed on days 1 and 8. The animals are euthanized on Day 15. Urine and blood samples are collected on day 7 and 14. A clinical pathology assessment is made on day 14. Body weight is determined prior to the study, on the first day of administration, and 1 week prior to necropsy. Food consumption per group will be assessed daily. Blood samples are taken via the tail vein after 6 hours of fasting. The following blood serum analysis is performed: erythrocyte count, mean cell volume, packed cell volume, hemoglobin, mean cell hemoglobin concentration, mean cell hemoglobin, thrombocyte count, leucocyte count, differential white cell count with cell morphology reticulocyte count, sodium potassium chloride calcium, inorganic phosphorus, glucose, urea creatinine, total bilirubin, total cholesterol, triglycerides, alkaline phosphatase, alanine aminotransferase, aspartate aminotransferase, total protein albumin albumin/globulin ratio. Urinalysis are performed α-GST, β-2 Microglobulin, Calbindin, Clusterin, Cystatin C, KIM-1, Osteopontin, TIMP-1, VEGF, and NGAL. Seven analytes (Calbindin, Clusterin, GST-a, KIM-1, Osteopontin, TIMP-1, VEGF) will be quantified under Panel 1 (MILLIPLEX® MAP Rat Kidney Toxicity Magnetic Bead Panel 1, RKTX1 MAG-37K). Three analytes (β-2 Microglobulin, Cystatin C, Lipocalin-2/NGAL) will be quantified under Panel 2 (MILLIPLEX® MAP Rat Kidney Toxicity Magnetic Bead Panel 2, RKTX2MAG-37K). The assay for the determination of these biomarkers' concentration in rat urines is based on the Luminex xMAP® technology. Microspheres coated with anti-α-GST/β-2 microglobulin/calbindin/clusterin/cystacin C/KIM-1/osteopontin/TIMP-1/VEGF/NGAL antibodies are color-coded with two different fluorescent dyes. The following parameters are determined (Urine using the ADVIA 1650): Urine protein, urine creatinine. Quantitative parameters: volume, pH (using 10-Multistix SG test strips/Clinitek 500 urine analyzer), specific gravity (using a refractometer). Semi-quantitative parameters (using 10-Multistix SG test strips/Clinitek 500 urine analyzer): proteins, glucose, ketones, bilirubin, nitrites, blood, urobilinogen, cytology of sediment (by microscopic examination). Qualitative parameters: Appearance, color. After sacrifice, the body weight and kidney, liver and spleen weight are determined and organ to body weight ratio calculated. Kidney and liver samples will be taken and either frozen or stored in formalin. Microscopic analysis is performed.

The rat safety study was performed at CiToxLabs, France. Male Wistar rats (n=4/group) were selected for the study as the Wistar Han rats in the used study set-up (dose range and time course) have previously been demonstrated to predict renal (and to some extent hepatic) toxicity in humans. The animals were injected s.c. Day 1 and Day 8 with conjugated LNA compounds (at 10 mg/kg), or corresponding unconjugated “parent compound” (at 40 mg/kg). Urine was collected Day 7 and Day 14 and kept on ice until analysis. Urine samples were centrifuged (approx. 380 g, 5 min, at +4° C.) and a panel of urinary injury markers analyzed with a multiplex assay based on the Luminex xMAP® technology. Out of the panel of urinary kidney injury markers in the study KIM-1 (kidney injury marker 1) demonstrated the largest dynamic range and most clear signal, as has recently been described for KIM-1 in a meta-analysis of urinary kidney injury markers (Vlasakova et al, Evaluation of the Relative Performance of Twelve Urinary Biomarkers for Renal Safety across Twenty Two Rat Sensitivity and Specificity Studies Toxicol. Sci. Dec. 21, 2013). The results are shown in table9

TABLE 9
The Kim marker results

| | Urine Kim 1 mean (2) |
|---|---|
| SEQ ID NO 28 10 mg/kg × 2 | 13.5 |
| SEQ ID NO 20 10 mg/kg × 2 | 103 |

Neither compound gave concerning levels of kim-1 in the rat urine, but the SEQ ID NO 2 cholesterol conjugate (SEQ ID NO 28) gave a lower average kim-1 level than the SEQ ID NO 1 GalNAc conjugate (SEQ ID NO 20). Please note, though, that urinary kim-1 protein levels for SEQ ID NO 28 and SEQ ID NO 20 still are low compared to kim-1 levels in urine from rats displaying clear tubular toxicity as demonstrated by kidney histology analysis at the same time point.

### Example 6

### ApoB Targeting Compounds with FAM Label Conjugates

FAM-labelled antisense oligomers (ASOs) with different DNA/PO-linkers as shown in table 4 were subjected to in vitro cleavage either with S1 nuclease extract, Liver or kidney homogenates or Serum.

S1 Nuclease Cleavage:

FAM-labeled oligonucleotides 100 μM with different DNA/PO-linkers were subjected to in vitro cleavage by S1 nuclease in nuclease buffer (60 U pr. 100 μL) for 20 and 120 minutes (see table below). The enzymatic activity was stopped by adding EDTA to the buffer solution. The solutions were then subjected to AIE HPLC analyses on a Dionex Ultimate 3000 using an Dionex DNApac p-100 column and a gradient ranging from 10 mM-1 M sodium perchlorate at pH 7.5. The content of cleaved and non-cleaved oligonucleotide was determined against a standard using both a fluorescence detector at 615 nm and a uv detector at 260 nm. The results are shown in Table 10.

TABLE 10
cleavage of phosphodiester linkages using nuclease

| SEQ ID | Linker | % cleaved | % cleaved |
|---|---|---|---|
| NO | sequence | after 20 min S1 | after 120 min S1 |
| 13 | — | 2 | 5 |
| 11 | a | 29.1 | 100 |
| 10 | ca | 40.8 | 100 |
| 9 | tca | 74.2 | 100 |
| 12 | gac | 22.9 | n.d |

Conclusion:

The PO linkers (or region B as referred to herein) results in the conjugate (or group C) being cleaved off, and both the length and/or the sequence composition of the linker can be used to modulate susceptibility to nucleolytic cleavage of region B. The Sequence of DNA/PO-linkers can modulate the cleavage rate as seen after 20 min in Nuclease 51 extract Sequence selection for region B (e.g. for the DNA/PO-linker) can therefore also be used to modulate the level of cleavage in serum and in cells of target tissues.

Cleavage by Homogenates and Serum:

Liver and kidney homogenates and Serum were spiked with oligonucleotide SEQ ID NO 9 to concentrations of 200 μg/g tissue (see table below). Liver and kidney samples collected from NMRI mice were homogenized in a homogenisation buffer (0.5% Igepal CA-630, 25 mM Tris pH 8.0, 100 mM NaCl, pH 8.0 (adjusted with 1 N NaOH). The homogenates were incubated for 24 hours at 37° C. and thereafter the homogenates were extracted with phenol-chloroform. The content of cleaved and non-cleaved oligonucleotide in the extract from liver and kidney and from the serum was determined against a standard using the above HPLC method. The results are shown in table 11.

TABLE 11
cleavage of phosphodiester linkages using homogenates

| | | % cleaved after | % cleaved after | |
|---|---|---|---|---|
| | Linker | 24 hrs liver | 24 hrs kidney | % cleaved after |
| Seq ID | Sequence | homogenate | homogenate | 24 hours in serum |
| 9 | tca | 83 | 95 | 0 |

Conclusion:

The PO linkers (or region B as referred to herein) results in cleavage of the conjugate (or group C) from the oligonucleotide in liver or kidney homogenate, but not in serum. Note: cleavage in the above assays refers to the cleavage of the cleavable linker, the oligomer or region A should remain functionally intact.

The susceptibility to cleavage in the assays shown in Example 7 may be used to determine whether a linker is biocleavable or physiologically labile.

### Example 7

### Knock Down of ApoB mRNA, Tissue Content, and Serum Total Cholesterol with GalNAc-Conjugates In Vivo

TABLE 12
Compounds

| SEQ | Seq | Cleavable | Conjugate |
|---|---|---|---|
| ID NO | (5′-3′) (A) | Linker (B) | (C) |
| 3 | GₛCₛaₛtₛtₛgₛgₛ | no | No |
| | tₛaₛtₛTₛCₛA | | |
| 30 | GₛCₛaₛtₛtₛgₛaₛ | | GalNAc cluster |
| | tₛaₛtₛTₛCₛA | | Conj1a |
| 20 | GₛCₛaₛtₛtₛgₛaₛ | | GalNAc cluster |
| | tₛaₛtₛTₛCₛA | | Conj2a |
| 7 | GₛCₛaₛtₛtₛgₛgₛ | 2PO-DNA | cholesterol |
| | tₛaₛtₛTₛCₛA | (5′ca3′) | |

Region A: Capital letters are LNA nucleosides (such as beta-D-oxy LNA), lower case letters are DNA nucleosides. Subscript s represents a phosphorothioate internucleoside linkage. LNA cytosines are optionally 5-methyl cytosine. Region B: The 2PO linker is 5′ to the sequence region A, and comprises two DNA nucleosides indicated in ( ) linked by phosphodiester linkage, with the internucleoside linkage between the 3′ DNA nucleoside of region B and the 5′ LNA nucleoside of region A also being phosphodiester. A linkage group (Y) in the form of a C6 linker (not shown in the table) has been used to link the conjugate group to region B (SEQ ID NO 7), or to region A (SEQ ID NO 20 and 30).

C57BL6/J mice were injected either iv or sc with a single dose saline or 0.25 mg/kg unconjugated LNA-antisense oligonucleotide (SEQ ID NO 3) or equimolar amounts of LNA antisense oligonucleotides conjugated to GalNAc1 (SEQ ID NO 30), GalNAc2 (SEQ ID NO 20), or cholesterol (2PO) (SEQ ID NO 7) and sacrificed at days 1-7 according to the table below (experimental design).

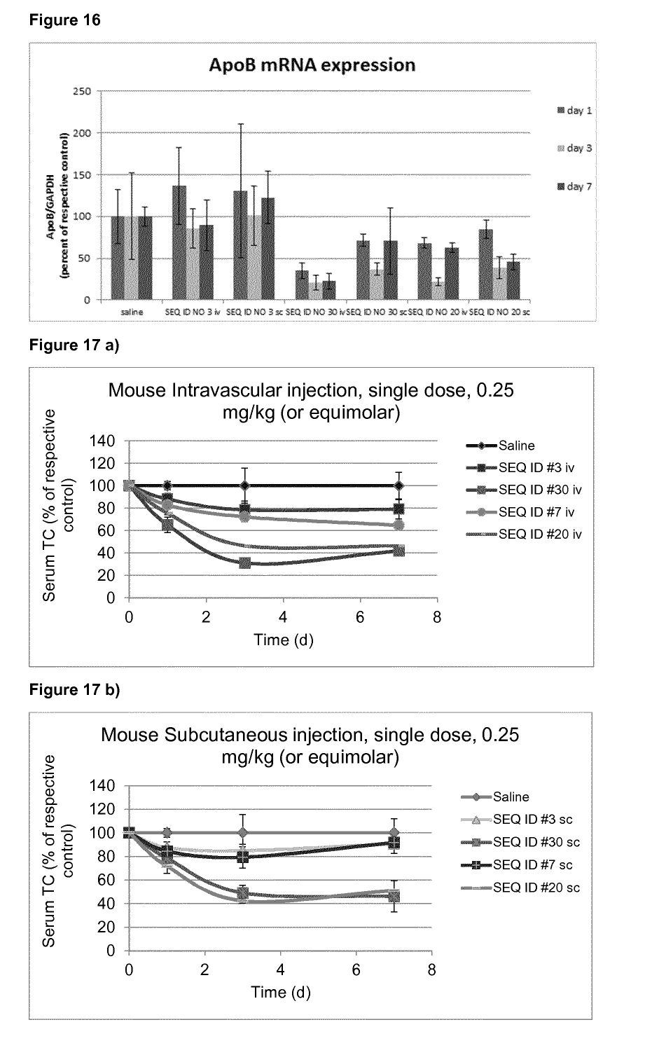

RNA was isolated from liver and kidney and subjected to qPCR with ApoB specific primers and probe to analyze for ApoB mRNA knockdown. The oligonucleotide content was measured using ELISA method and total cholesterol in serum was measured. The results are shown in FIGS. 16 and 17.

Materials and Methods:

Experimental Design:

| | | | | Compound | Conc. at | | | |
|---|---|---|---|---|---|---|---|---|
| Group | Animal | No. of | Animal strain/ | Dose level | dose vol. | Adm. | Dosing | Sacrifice |
| no. | id no. | Animals | gender/feed | per day | 10 ml/kg | Route | day | day |
| 1 | 1-3 | 3 | C57BL/6J/♀/Chow | Saline | — | i.v | 0 | 1 |
| 2 | 4-6 | 3 | C57BL/6J/♀/Chow | SEQ ID NO 3 | 0.025 mg/ml | i.v | 0 | 1 |
| | | | | 0.25 mg/kg | | | | |
| 3 | 7-9 | 3 | C57BL/6J/♀/Chow | SEQ ID NO 3 | 0.025 mg/ml | s.c | 0 | 1 |
| | | | | 0.25 mg/kg | | | | |
| 4 | 10-12 | 3 | C57BL/6J/♀/Chow | SEQ ID NO 30 | 0.036 mg/ml | i.v | 0 | 1 |
| | | | | 0.36 mg/kg | | | | |
| 5 | 13-15 | 3 | C57BL/6J/♀/Chow | SEQ ID NO 30 | 0.036 mg/ml | s.c | 0 | 1 |
| | | | | 0.36 mg/kg | | | | |
| 6 | 16-18 | 3 | C57BL/6J/♀/Chow | SEQ ID NO 7 | 0.032 mg/ml | i.v | 0 | 1 |
| | | | | 0.32 mg/kg | | | | |
| 7 | 19-21 | 3 | C57BL/6J/♀/Chow | SEQ ID NO 7 | 0.032 mg/ml | s.c | 0 | 1 |
| | | | | 0.32 mg/kg | | | | |
| 8 | 22-24 | 3 | C57BL/6J/♀/Chow | SEQ ID NO 20 | 0.034 mg/ml | i.v | 0 | 1 |
| | | | | 0.34 mg/kg | | | | |
| 9 | 25-27 | 3 | C57BL/6J/♀/Chow | SEQ ID NO 20 | 0.034 mg/ml | s.c | 0 | 1 |
| | | | | 0.34 mg/kg | | | | |
| 10 | 28-30 | 3 | C57BL/6J/♀/Chow | Saline | — | i.v | 0 | 3 |
| 11 | 31-33 | 3 | C57BL/6J/♀/Chow | SEQ ID NO 3 | 0.025 mg/ml | i.v | 0 | 3 |
| | | | | 0.25 mg/kg | | | | |
| 12 | 34-36 | 3 | C57BL/6J/♀/Chow | SEQ ID NO 3 | 0.025 mg/ml | s.c | 0 | 3 |
| | | | | 0.25 mg/kg | | | | |
| 13 | 37-39 | 3 | C57BL/6J/♀/Chow | SEQ ID NO 30 | 0.036 mg/ml | i.v | 0 | 3 |
| | | | | 0.36 mg/kg | | | | |
| 14 | 40-42 | 3 | C57BL/6J/♀/Chow | SEQ ID NO 30 | 0.036 mg/ml | s.c | 0 | 3 |
| | | | | 0.36 mg/kg | | | | |
| 15 | 43-45 | 3 | C57BL/6J/♀/Chow | SEQ ID NO 7 | 0.032 mg/ml | i.v | 0 | 3 |
| | | | | 0.32 mg/kg | | | | |
| 16 | 46-48 | 3 | C57BL/6J/♀/Chow | SEQ ID NO 7 | 0.032 mg/ml | s.c | 0 | 3 |
| | | | | 0.32 mg/kg | | | | |
| 17 | 49-51 | 3 | C57BL/6J/♀/Chow | SEQ ID NO 20 | 0.034 mg/ml | i.v | 0 | 3 |
| | | | | 0.34 mg/kg | | | | |
| 18 | 52-54 | 3 | C57BL/6J/♀/Chow | SEQ ID NO 20 | 0.034 mg/ml | s.c | 0 | 3 |
| | | | | 0.34 mg/kg | | | | |
| 19 | 55-57 | 3 | C57BL/6J/♀/Chow | Saline | — | i.v | 0 | 7 |
| 20 | 58-60 | 3 | C57BL/6J/♀/Chow | SEQ ID NO 3 | 0.025 mg/ml | i.v | 0 | 7 |
| | | | | 0.25 mg/kg | | | | |
| 21 | 61-63 | 3 | C57BL/6J/♀/Chow | SEQ ID NO 3 | 0.025 mg/ml | s.c | 0 | 7 |
| | | | | 0.25 mg/kg | | | | |
| 22 | 64-66 | 3 | C57BL/6J/♀/Chow | SEQ ID NO 30 | 0.036 mg/ml | i.v | 0 | 7 |
| | | | | 0.36 mg/kg | | | | |
| 23 | 67-69 | 3 | C57BL/6J/♀/Chow | SEQ ID NO 30 | 0.036 mg/ml | s.c | 0 | 7 |
| | | | | 0.36 mg/kg | | | | |
| 24 | 70-72 | 3 | C57BL/6J/♀/Chow | SEQ ID NO 7 | 0.032 mg/ml | i.v | 0 | 7 |
| | | | | 0.32 mg/kg | | | | |
| 25 | 73-75 | 3 | C57BL/6J/♀/Chow | SEQ ID NO 7 | 0.032 mg/ml | s.c | 0 | 7 |
| | | | | 0.32 mg/kg | | | | |
| 26 | 76-78 | 3 | C57BL/6J/♀/Chow | SEQ ID NO 10 | 0.034 mg/ml | i.v | 0 | 7 |
| | | | | 0.34 mg/kg | | | | |
| 27 | 79-81 | 3 | C57BL/6J/♀/Chow | SEQ ID NO 20 | 0.034 mg/ml | s.c | 0 | 7 |
| | | | | 0.34 mg/kg | | | | |

Dose Administration.

C57BL/6JBorn female animals, app. 20 g at arrival, were dosed with 10 ml per kg BW (according to day 0 bodyweight) i.v. or s.c. of the compound formulated in saline or saline alone according to the table above.

Sampling of Liver and Kidney Tissue.

The animals were anaesthetized with 70% CO₂— 30% O₂ and sacrificed by cervical dislocation according to the above table. One half of the large liver lobe and one kidney were minced and submerged in RNAlater. The other half of liver and the other kidney was frozen and used for tissue analysis.

Total RNA Isolation and First Strand Synthesis.

Total RNA was extracted from maximum 30 mg of tissue homogenized by bead-milling in the presence of RLT-Lysis buffer using the Qiagen RNeasy kit (Qiagen cat. no. 74106) according to the manufacturer's instructions. First strand synthesis was performed using Reverse Transcriptase reagents from Ambion according to the manufacturer's instructions.

For each sample 0.5 μg total RNA was adjusted to (10.8 μl) with RNase free H₂O and mixed with 2 μl random decamers (50 μM) and 4 μl dNTP mix (2.5 mM each dNTP) and heated to 70° C. for 3 min after which the samples were rapidly cooled on ice. 2 μl 10× Buffer RT, 1 μl MMLV Reverse Transcriptase (100 U/μl) and 0.25 μl RNase inhibitor (10 U/μl) were added to each sample, followed by incubation at 42° C. for 60 min, heat inactivation of the enzyme at 95° C. for 10 min and then the sample was cooled to 4° C. cDNA samples were diluted 1: 5 and subjected to RT-QPCR using Taqman Fast Universal PCR Master Mix 2x (Applied Biosystems Cat #4364103) and Taqman gene expression assay (mApoB, Mn01545150_m1 and mGAPDH #4352339E) following the manufacturers protocol and processed in an Applied Biosystems RT-qPCR instrument (7500/7900 or ViiA7) in fast mode. Oligonucleotide content in liver and kidney was measured by sandwich ELISA method.

Serum Cholesterol Analysis

Immediately before sacrifice retro-orbital sinus blood was collected using S-monovette Serum-Gel vials (Sarstedt, Nümbrecht, Germany) for serum preparation. Serum was analyzed for total cholesterol using ABX Pentra Cholesterol CP (Triolab, Brondby, Denmark) according to the manufacturer's instructions.

Conclusions:

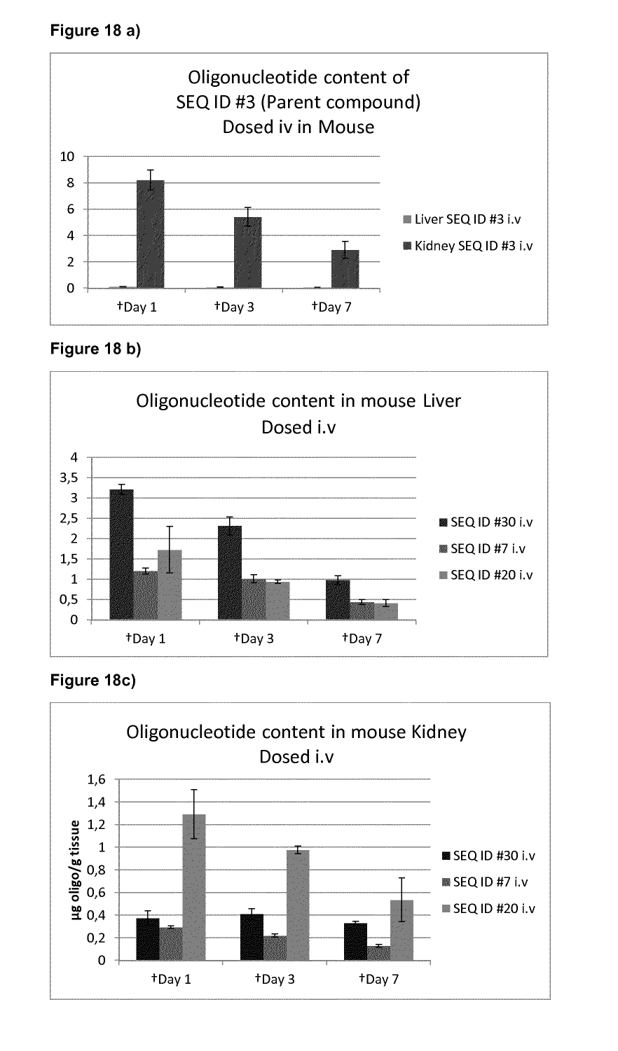

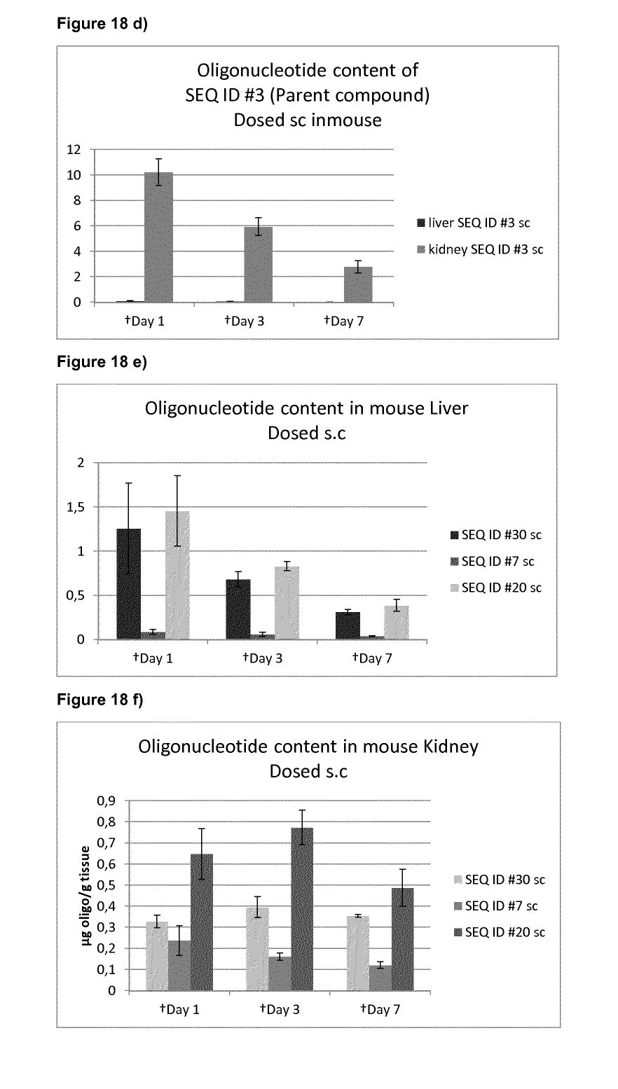

GalNAc1 and GalNAc2 conjugated to an ApoB LNA antisense oligonucleotide (SEQ ID NO 30 and 20) showed knock down of ApoB mRNA better than the unconjugated ApoB LNA (FIG. 16). For GalNAc 1 conjugate (SEQ ID NO 30) is seems that iv dosing is better than sc dosing which is surprising since the opposite has been reported for another GalNAc clusters (Alnylam, 8th Annual Meeting of the Oligonucleotide Therapeutics Society). The total cholesterol (TC) data show how the GalNAc cluster conjugates (SEQ ID NO 30 and 20) gives better effect than the unconjugated (SEQ ID NO 3) and the cholesterol conjugated compounds (SEQ ID NO 7) both at iv and sc administration (FIG. 17, a and b). The tissue content of the oligonucleotides (FIG. 18, a-f) shows how the conjugates enhances the uptake in liver while giving less uptake in kidney compared to the parent compound. This holds for both iv and sc administration. When dosing iv the GalNAc 1 (SEQ ID NO 30) gives very much uptake in liver when compared to GalNAc 2 (SEQ ID NO 20) but since activity is good for both compounds the GalNAc 2 conjugate appears to induce a higher specific activity than GalNAc 1 conjugate indicating that GalNAc conjugates without the pharmacokinetic modulator may be particularly useful with LNA antisense oligonucleotides.

### Example 8

### Knock Down of ApoB mRNA and Serum Total Cholesterol with GalNAc-Conjugates In Vivo

To explore duration of action of different conjugation moieties on the activity of an ApoB compound, SEQ ID NO 27 was conjugated to either cholesterol+biocleavable linker (two DNA nucleotides with phosphodiester backbone (PO); SEQ ID 28) or GalNAc cluster (SEQ ID NO 29). See Table 6 for more construct details. C57BL6In mice were injected i.v. with saline control or with a single dose of 0.1, 0.25 or 1.0 mg/kg SEQ ID NO 27, SEQ ID NO 28, or SEQ ID NO 29, respectively (conjugated dosed equimolar to the unconjugated SEQ ID NO 27). Effect was monitored by analysis of plasma cholesterol days 4, 7, 10, 14, and 24 after single injection of respective compound. Groups of four animals were sacrificed day 4, 14, and 24 after single injection and RNA was isolated from liver and kidney samples and analysed for ApoB mRNA expression as described in example 7. Results are shown in table 13.

TABLE 13
Liver ApoB mRNA levels

| | Day after single i.v. injection | | | | | | | | |
|---|---|---|---|---|---|---|---|---|---|
| | Day 4 | | | Day 14 | | | Day 24 | | |
| | 0.1 | 0.25 | 1.0 | 0.1 | 0.25 | 1.0 | 0.1 | 0.25 | 1.0 |
| Compound | mg/kg | mg/kg | mg/kg | mg/kg | mg/kg | mg/kg | mg/kg | mg/kg | mg/kg |
| SEQ ID 27 | 93 ± 10 | 86 ± 9 | 72 ± 7 | 113 ± 9 | 89 ± 11 | 96 ± 6 | 100 ± 2 | 94 ± 6 | 82 ± 11 |
| SEQ ID 28 | 89 ± 8 | 56 ± 9 | 21 ± 2 | 97 ± 7 | 92 ± 8 | 58 ± 12 | 99 ± 13 | 84 ± 16 | 71 ± 11 |
| SEQ ID 29 | 52 ± 8 | 23 ± 3 | 5 ± 0 | 86 ± 9 | 72 ± 11 | 42 ± 6 | 86 ± 12 | 77 ± 11 | 56 ± 11 |

The data are normalized to GAPDH and presented as percent of saline treated animals sacrificed at the same time point. Data are mean±SD.

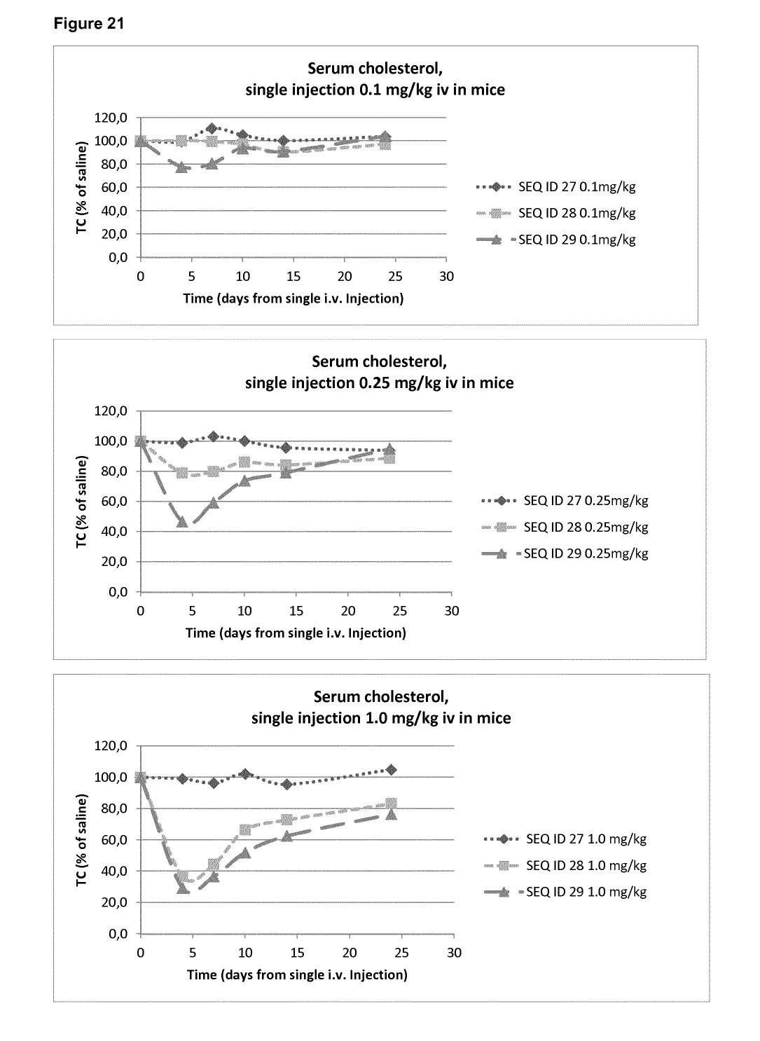

Total serum cholesterol was analysed as described in example 7 on days 0, 4, 7, 10, 14 and 24. The results are shown in FIG. 21.

Cholesterol and GalNAc conjugated versions of oligonucleotide with SEQ ID NO 27 both show increased down regulation of ApoB mRNA in the liver compared to the unconjugated oligonucleotide. In particular the GalNAc conjugation results in an improved effect when compared to both the unconjugated oligonucleotide and the cholesterol conjugation. The same effect is observed on total serum cholesterol levels, where both conjugates are quite efficient at the 1.0 mg/kg dose. When the dose is reduced the GalNAc conjugate (SEQ ID NO 29) appears to be more efficient when compared to both the unconjugated oligonucleotide and the cholesterol conjugation.

### Example 9

### Non-Human Primate Study; Multiple Injections s.c

The objective of this non-human primate study was to assess efficacy and safety of the anti-apoB compounds in a repeat administration setting, when compounds were administered by subcutaneous injection (s.c.). The compounds used in this study are SEQ ID NOs 27, 28, and 29, prepared in sterile saline (0.9%) at an initial concentration of 0.625 and 2.5 mg/ml).

Male monkeys of at least 24 months old were used, and given free access to tap water and 180 g of OWM(E) SQC SHORT expanded diet (Dietex France, SDS, Saint Gratien, France) was distributed daily per animal. In addition, fruit or vegetables was given daily to each animal. The animals was acclimated to the study conditions for a period of at least 14 days before the beginning of the treatment period. During this period, pre-treatment investigations was performed. The animals were dosed s.c. once a week for four weeks at a dose of 0.1 mg/kg or 0.5 mg/kg/injection, with four injections total over a period of four weeks with injections on day 1, day 8, day 15 and day 22. The dose volume was be 0.4 mL/kg/injection. Four animals were used per group except for the group with the unconjugated oligomer (SEQ ID NO 27) which only contained 2 animals. After the fourth and final dose animals were observed for a week (day 29) after which two of the animals were sacrificed in order to study liver ApoB transcript regulation, lipid parameters, liver and kidney histology, and liver and kidney tissue distribution. The remaining two animals were followed for another 7 weeks. Day 1 corresponds to the first day of the treatment period. Clinical observations and body weight and food intake (per group) was recorded prior to and during the study.

Blood and tissues were sampled and analysed at the following time points:

| Study Day | Parameters |
|---|---|
| −10 | L, Apo-B, OA |
| −5 | LSB, L, Apo-B, OA |
| −1 | RCP, L, Apo-B, PK, OA |
| 1 | Dosing |
| 8 pre-dose | LSB, L, Apo-B, PK, OA |
| 8 | Dosing |
| 15 pre-dose | LSB, L, Apo-B, PK, OA |
| 15 | Dosing |
| 22 pre-dose | LSB, L, Apo-B, PK, OA |
| 22 | Dosing |
| 29 | RCP, PK, OA + necropsy main |
| 36 (recovery animals) | LSB, L, Apo-B, PK, OA |
| 43 (recovery animals) | RCP, PK, Apo-B, PK, OA |
| 50 (recovery animals) | LSB, L, Apo-B, PK, OA |
| 57 (recovery animals) | LSB, L, Apo-B, PK, OA |
| 64 (recovery animals) | LSB, L, Apo-B, PK, OA |
| 71 (recovery animals) | LSB, L, Apo-B, PK, OA |
| 78 (recovery animals) | RCP, L, Apo-B, PK, OA + necropsy recovery |

RCP: routine clinical pathology,
LSB: liver safety biochemistry,
PK: pharmacokinetics,
OA: other analyses,
L: lipids

Blood (approximately 1.0 mL) was taken into lithium heparin tubes (using the ADVIA 1650 blood biochemistry analyser) analyzing sodium, potassium, chloride, calcium, inorganic phosphorus, glucose, HDL-C, LDL-C, urea, creatinine, total bilirubin (TBIL), total cholesterol, triglycerides, alkaline phosphatase (ALP), alanine aminotransferase (ALAT), aspartate aminotransferase (ASAT), creatine kinase, gamma-glutamyl transferase (GGT), lactate dehydrogenase, total protein, albumin, albumin/globulin ratio.

Analysis of Blood:

Blood samples for ApoB analysis was collected from Group 1-16 animals only (i.e. animals treated with anti-ApoB compounds) on Days −8, −1, 4, 8, 15, 22, 29, 36, 43 and 50. Venous blood (approximately 2 mL) was collected from an appropriate vein in each animal into a Serum Separating Tube (SST) and allowed to clot for at least 60±30 minutes at room temperature. Blood was centrifuged at 1000 g for 10 minutes under refrigerated conditions (set to maintain+4° C.). The serum was transferred into 3 individual tubes and stored at −80° C. until analysis of ApoB protein by ELISA.

Other Analysis described in WO2010/142805 are qPCR, ApoB mRNA analysis. Other analysis includes, serum Lp(a) analysis with ELISA (Mercodia No. 10-1106-01), tissue and serum oligonucleotide analysis (drug content), Extraction of samples, standard—and QC-samples, Oligonucleotide content determination by ELISA.

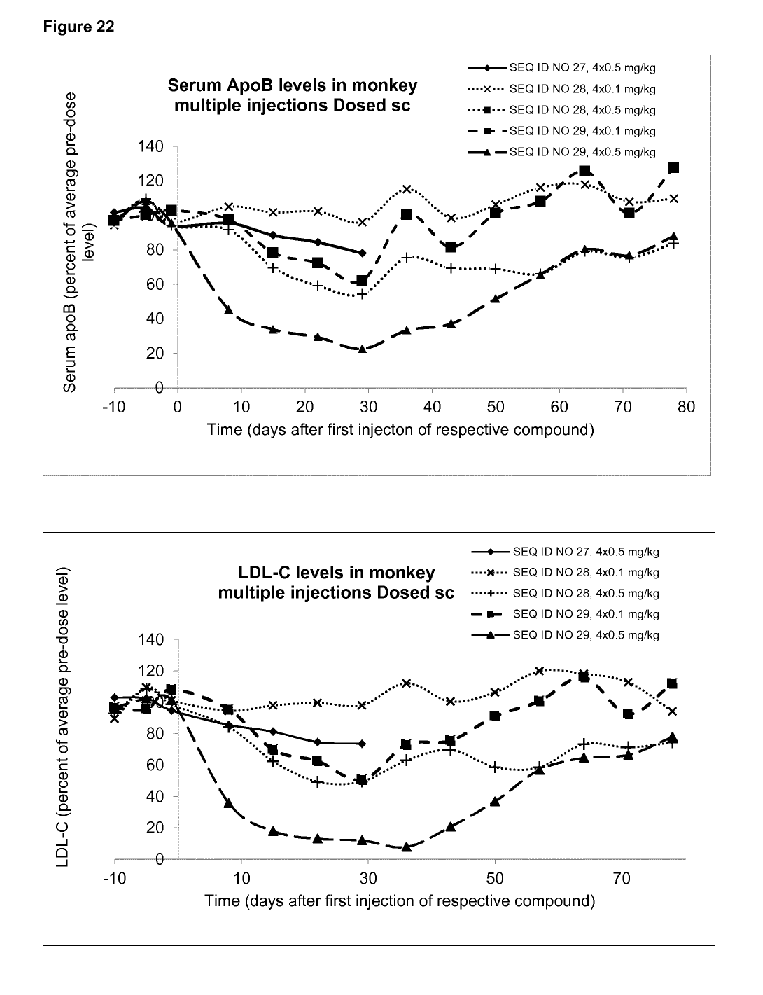

Data for SEQ ID NO 27, SEQ ID NO 28 and SEQ ID NO 29 are shown in FIG. 22. From this, it can be seen that both conjugated oligomers (SEQ ID NO 28 and 29) were more effective at equivalent dose than the unconjugated oligomer (SEQ ID NO 27) at the time of the second injection. In particular, the SEQ ID NO 27-GalNAc compound (SEQ ID NO 29) gave a rapid, dose dependent, and highly effective down regulation of serum ApoB and LDL-C. This illustrated that just as in the single dose experiment described in Example 5, the GalNAc conjugation of SEQ ID NO 27 was more effective than the cholesterol-conjugation of SEQ ID NO 27, i.e. efficacy of SEQ ID NO 29 is superior to efficacy of SEQ ID NO 28. This is an indication that the GalNAc compound may be dosed comparatively infrequently and at a lower dosage, as compared to both the unconjugated parent compound, and compounds using alternative conjugation technology, such as cholesterol conjugation (such as SEQ ID 28). The SEQ ID NO 27-GalNAc compound (SEQ ID NO 29) also showed a very long lasting effect after the last injection at day 22. Even 8 weeks after the last treatment the ApoB and LDL cholesterol levels in serum had not returned to the baseline before treatment. The same was the case for the SEQ ID NO 27-Cholesterol compound (SEQ ID NO 28) This indicates a long pharmacodynamic half-life of these conjugated compounds.

Liver and kidney oligonucleotide content was analysed one week after last injection, i.e. day 29 of the study. Oligonucleotide content was analysed using hybridization ELISA (essentially as described in Lindholm et al, Mol Ther. 2012 February; 20(2):376-81), using SEQ ID NO 27 to prepare a standard curve for samples from animals treated with SEQ ID NO 27, SEQ ID 28, and SEQ ID NO 29, after having controlled that there was no change in result if the (conjugated) SEQ ID NO 28 or SEQ ID NO 29 were used for preparation of standard curve. The results are shown in Table 14

TABLE 14
Oligonucleotide content in tissues one week after last injection

| | Liver (μg | Kidney (μg | |
|---|---|---|---|
| | oligonucleotide/ | oligonucleotide/ | |
| | g wet tissue) | g wet tissue) | Liver/ |
| | Average | Average | kidney ratio |
| SEQ ID NO 27, 4 × | <0.05 | 32.1 | <0.0016 |
| 0.5 mg/kg | | | |
| SEQ ID NO 28, 4 × | <0.05 | 6.7 | <.0075 |
| 0.1 mg/kg | | | |
| SEQ ID NO 28, 4 × | <0.05 | 40.6 | <0.0012 |
| 0.5 mg/kg | | | |
| SEQ ID NO 29, 4 × | 0.91 | 5.7 | 0.16 |
| 0.1 mg/kg | | | |
| SEQ ID NO 29, 4 × | 14.4 | 33.4 | 0.43 |
| 0.5 mg/kg | | | |

As illustrated in the table above, SEQ ID NO 29 (GalNAc conjugation) demonstrates a strong shift in liver/kidney distribution compared with both the unconjugated compound (SEQ ID NO 27) and cholesterol conjugated compound (SEQ ID NO 28) after four weekly s.c. injections of equimolar amounts of the respective compounds. A shift to a higher liver/kidney ratio, with retained or improved efficacy, is expected to result in improved safety profile for the compound with higher vs. lower liver/kidney ratio of oligonucleotide tissue content.

1. An antisense oligonucleotide conjugate comprising an oligomer with the oligonucleotide motif of SEQ ID NO 2 joined with a conjugate moiety (region C), where the conjugate moiety comprises one or more N-acetylgalactosamine moieties.

2. The antisense oligonucleotide conjugate according to claim 1, wherein the oligomer comprises at least 2 affinity enhancing nucleotide analogues selected from the group consisting of: Locked Nucleic Acid (LNA) units; 2′-O-alkyl-RNA units, 2′-OMe-RNA units, 2′-amino-DNA units, and 2′-fluoro-DNA units.

3. The antisense oligonucleotide conjugate according to claim 1, wherein the oligomer corresponds to SEQ ID NO 27: 5′ GsTstsgsascsascstsgsTsC 3′, wherein capital letters represent beta-D-oxy LNA, lower case letters represent DNA nucleosides, LNA cytosines are 5-methyl cytosine, and all internucleoside linkages are phosphorothioate indicated by s.

4. The antisense oligonucleotide conjugate according to claim 1, wherein the oligomer is capable of down regulating the expression of ApoB in a cell which is expressing ApoB.

5. The antisense oligonucleotide conjugate according to claim 1, wherein said conjugate moiety is joined to said oligomer, via a cleavable linker (B).

6. The antisense oligonucleotide conjugate according to claim 5, wherein the cleavable linker comprises a cleavable lysine linker.

7. The antisense oligonucleotide conjugate according to claim 1, wherein the conjugate moiety comprises 1 to 3 N-acetylgalactosamine moiety(s).

8. The antisense oligonucleotide conjugate according to claim 1, wherein the oligonucleotide conjugate comprises a linker Y which covalently links the conjugate moiety to the oligomer.

9. The antisense oligomer conjugate according to claim 8, wherein the linker region Y comprises a fatty acid, such as a C6 linker.

10. The antisense oligonucleotide according to claim 1, wherein conjugate moiety comprises a PEG spacer between the N-acetylgalactosamine moiety(s) and the linker (B and/or Y) or the oligomer.

11. The antisense oligonucleotide conjugate according to claim 1, wherein the conjugate moiety comprises three N-acetylgalactosamine moieties each independently linked via a PEG spacer to a cleavable di-lysine linker.

12. The antisense oligonucleotide conjugate according to claim 1, wherein the conjugate comprises a conjugate moiety selected from the group consisting of Conj1, Conj2, Conj3, Conj4, Conj1 a, Conj2a, Conj3a and Conj4a.

13. The antisense oligonucleotide conjugate according to claim 1, which consists of SEQ ID NO 29 or SEQ ID NO 31.

14. A pharmaceutical composition comprising the antisense oligonucleotide conjugate according to claim 1, and a pharmaceutically acceptable diluent, carrier, salt or adjuvant.

15. The antisense oligonucleotide conjugate or pharmaceutical composition according to claim 1, for use as a medicament.

16. An in vivo or in vitro method for the inhibition of ApoB in a cell which is expressing ApoB, said method comprising administering an oligonucleotide conjugate or pharmaceutical composition according to claim 1 to said cell so as to inhibit ApoB in said cell.
